SEQUENCE LISTING (9 sequences)

SEQ ID NO: 1 - protein, 449 aa, Artificial Sequence
  synthetic humanized TRA-8 anti-human death receptor 5 (DR5) monoclonal antibody heavy chain
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial Sequence
  synthetic humanized TRA-8 anti-human death receptor 5 (DR5) monoclonal antibody light chain
DIQMTQSPSSLSASVGDRVTITCKASQDVGTAVAWYQQKPGKAPKLLIYWASTRHTGVPSRFSGSGSGTDFTLTISSLQPEDFATYYCQQYSSYRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 5 aa, Artificial Sequence
  synthetic humanized TRA-8 anti-human death receptor 5 (DR5) monoclonal antibody heavy chain CDR1
SYVMS

SEQ ID NO: 4 - protein, 17 aa, Artificial Sequence
  synthetic humanized TRA-8 anti-human death receptor 5 (DR5) monoclonal antibody heavy chain CDR2
TISSGGSYTYYPDSVKG

SEQ ID NO: 5 - protein, 10 aa, Artificial Sequence
  synthetic humanized TRA-8 anti-human death receptor 5 (DR5) monoclonal antibody heavy chain CDR3
RGDSMITTDY

SEQ ID NO: 6 - protein, 11 aa, Artificial Sequence
  synthetic humanized TRA-8 anti-human death receptor 5 (DR5) monoclonal antibody light chain CDR1
KASQDVGTAVA

SEQ ID NO: 7 - protein, 7 aa, Artificial Sequence
  synthetic humanized TRA-8 anti-human death receptor 5 (DR5) monoclonal antibody light chain CDR2
WASTRHT

SEQ ID NO: 8 - protein, 8 aa, Artificial Sequence
  synthetic humanized TRA-8 anti-human death receptor 5 (DR5) monoclonal antibody light chain CDR3
QQYSSYRT

SEQ ID NO: 9 - protein, 4 aa, Artificial Sequence
  synthetic peptidyl linker cleavable by thiol-dependent protease cathepsin-B
GFLG

(19) United States
(12) Patent Application Publication
(10) Pub. No.: US 2011/0070248 A1
(43) Pub. Date: Mar. 24, 2011

(54) DR5 LIGAND DRUG CONJUGATES

(75) Inventors: Kimihisa Ichikawa, Tokyo (JP); Kosaku Fujiwara, Tokyo (JP); Hiroko Yoshida, Tokyo (JP); Ayumi Yada, Tokyo (JP)

(73) Assignees: Seattle Genetics, Inc., Bothell, WA (US); Daiichi Sankyo Co., Ltd., Tokyo (JP)

(21) Appl. No.: 12/889,306

(22) Filed: Sep. 23, 2010

Related U.S. Application Data
(60) Provisional application No. 61/245,462, filed on Sep. 24, 2009.

(51) Int. Cl. A61K 39/395; C07K 16/28; A61P 35/00

(52) U.S. Cl. 424/178.1; 530/391.7; 530/391.9

(57) ABSTRACT

Ligand Drug Conjugates are provided having a DR5 binding moiety attached via linking groups and/or spacers to a therapeutic agent and are effective in treatment of various cancers.

### CROSS-REFERENCES TO RELATED APPLICATIONS

This application claims priority benefit of U.S. Provisional Application Ser. No. 61/245,462, filed Sep. 24, 2009, the contents of which are incorporated herein by reference in their entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Cell surface receptors involved in apoptosis induction, such as death domain-containing receptors, multimerize on the membrane of cells due to ligand binding and biologically trigger induction of apoptotic signals in the cells (Cell Death and Differentiation, 10:66-75 (2003)). Examples of such cell surface receptors include the tumor necrosis factor (hereinafter referred to as TNF)-related apoptosis-inducing ligand (hereinafter referred to as TRAIL) receptor family. TRAIL is a member of the TNF family of proteins, which includes Fas ligand and TNF-α (Wiley S R, et al. Immunity 1995 December; 3(6):673-82). These proteins are potent apoptosis inducers.

The receptors for the TNF family of proteins are characterized by a cysteine-rich repeat sequence in the extracellular domain. Among them, Fas, a receptor for Fas ligand, and TNF receptor I (hereinafter referred to as TNFRI), a receptor for TNF-α, are collectively referred to as death domain-containing receptors. These receptors have an intracellular domain essential for apoptotic signaling which is called the death domain and homologous to the Drosophila suicide gene, reaper (Golstein, P., et al. (1995) Cell. 81, 185-186, White, K, et al. (1994) Science 264, 677-683).

Five TRAIL receptors have been identified, and two (DR4 [TRAIL-R1] and DR5 [TRAIL-R2]) of them can induce apoptotic signaling while the other three (DcR1 [TRAIL-R3], DcR2 [TRAIL-R4], and osteoprotegerin [OPG]) do not induce apoptotic signaling. Like Fas and TNFRI, intracellular segments of both DR4 and DR5 contain a death domain and induce apoptotic signaling by way of pathways including Fas-associated death domain protein (hereinafter referred to as FADD) and caspase-8 (Chaudhary P M, et al. Immunity 1997 December; 7(6): 821-30; Schneider P, et al. Immunity 1997 December; 7(6): 831-36).

For the above Fas, TNFRI, DR4, and DR5, agonistic antibodies which bind respectively to these molecules have an apoptosis-inducing activity on cells having the target molecules on their surface (Journal of Cellular Physiology, 209: 1021-1028 (2006); Leukemia, Apl; 21 (4): 805-812 (2007); Blood, 99: 1666-1675 (2002); Cellular Immunology, January; 153 (1): 184-193 (1994)). The efficacy of these agonistic antibodies is enhanced by cross-linking with a secondary antibody or effector cells (Journal of Immunology, 149: 3166-3173 (1992); European Journal of Immunology, October; 23 (10): 2676-2681 (1993)).

An anti-DR5 antibody having capacity to bind to a cell surface receptor involved in apoptosis induction is currently under clinical development as a therapeutic, and is expected to reveal the therapeutic effects and kill the cells (cancer cells and immune disease-related cells) expressing the receptor in a specific and agonistic manner. The mechanism of action of this antibody is proposed to be mediated by cross-linking of the antibody molecules together to form multimers before or after the binding of the antibody to the receptor. Such multimerization of the antibody subsequently causes multimerization of the antigen receptor (namely, apoptosis induction). It appears that in vitro experiments, artificial cross-linking by addition of a secondary antibody against the antibody is required to enhance the activity of the antibody and that in vivo, cross-linking by Fc receptors on immune effector cells is a mechanism of action required to produce the activity of the antibody. Recently, attempts have been made to further enhance the original function of an antibody by altering structures of the antibody. For example, it is reported that removal of a specific carbohydrate structure on an antibody improves the affinity for Fc receptors. Such a mechanism of action suggests that non-internalizing antibodies to the cell surface receptor are preferred.

There remains a need, however, for methods of treating DR5 expressing cancers.

### BRIEF SUMMARY OF THE INVENTION

The present invention provides, inter alia, Ligand Drug Conjugates for targeted delivery of drug to DR5-expressing cells. The present inventors have conducted extensive studies and found that an antibody-drug conjugate containing an antibody that can induce apoptosis in cells has a more significant therapeutic effect on cancer than such antibody alone. By using the antibody-drug conjugate according to the present invention, the antibody itself exhibits an apoptosis-inducing effect and the drug conjugated to the antibody also exhibits a therapeutic effect. For these reasons, the antibody-drug conjugate has an effective therapeutic effect on patients who cannot be treated effectively by the antibody alone. The Ligand Drug Conjugates described herein have potent cytotoxic and/or cytostatic activity against cells expressing DR5, such as DR5-expressing cancer cells. In some embodiments, the Ligand Drug Conjugate has the formula:

L-(LU-D)ₚ (I)

wherein L is a Ligand unit, LU is a Linker unit and D is a Drug unit (or cytotoxic agent). The subscript p is an integer of from 1 to 20. Accordingly, the Ligand Drug Conjugates comprise a Ligand unit covalently linked to at least one Drug unit. The Drug units can be covalently linked directly or via a Linker unit (-LU-). The Ligand unit, described more fully below, is a DR5 binding agent, such as an anti-DR5 antibody. Accordingly, the present invention also provides methods for the treatment of, for example, various cancers. These methods encompass the use of Ligand Drug Conjugates wherein the Ligand unit is an anti-DR5 binding agent that specifically binds to DR5. The DR5 binding agent can be, for example, an anti-DR5 antibody, an anti-DR5 antigen-binding fragment, or other DR5 binding agent comprising the amino acid sequence of a humanized antibody heavy and/or light chain variable region, or derivative thereof.

### BRIEF DESCRIPTION OF THE DRAWINGS

FIGS. 1-11 provide the results for 11 cell lines evaluated with hTRA-8 Ligand Drug Conjugates of the present invention.

FIG. 12 illustrates the binding activity of hTRA-8 Ligand Drug Conjugates to human DR5 as compared to that of hTRA-8 (in an unconjugated form).

FIG. 13 illustrates that hTRA-8 Ligand Drug Conjugates did not show cytotoxicity against primary human hepatocytes as compared to hTRA-8.

FIGS. 14-26 provide in vivo results for the Ligand Drug Conjugates of the present invention.

FIG. 27 illustrates competition of anti-tumor activity of hTRA-8 Ligand Drug Conjugates in an A375 xenograft model.

FIG. 28 illustrates competition of anti-tumor activity of hTRA-8 Ligand Drug Conjugates in an HCT116 xenograft model.

FIG. 29 illustrates the in vivo anti-tumor efficacy of hTRA-8 Ligand Drug Conjugates in a JIMT-1 xenograft model.

FIG. 30 illustrates the in vivo anti-tumor efficacy of hTRA-8 Ligand Drug Conjugates in an MDA-MB-231 xenograft model.

FIG. 31 illustrates the in vivo anti-tumor efficacy of hTRA-8 Ligand Drug Conjugates in an A2780 xenograft model.

FIG. 32 illustrates the in vivo anti-tumor efficacy of hTRA-8 Ligand Drug Conjugates in an SK-OV-3 xenograft model.

FIG. 33 illustrates the in vivo life prolongation efficacy of hTRA-8 Ligand Drug Conjugates in U-937-inoculated mice.

FIG. 34 illustrates the in vivo life prolongation efficacy of hTRA-8 Ligand Drug Conjugates in MOLT-4-inoculated mice.

FIG. 35 illustrates the in vivo life prolongation efficacy of hTRA-8 Ligand Drug Conjugates in MOLM-14-inoculated mice.

FIG. 36 illustrates the in vivo life prolongation efficacy of hTRA-8 Ligand Drug Conjugates in MV-4-11-inoculated mice.

### DETAILED DESCRIPTION OF THE INVENTION

### Definitions and Abbreviations

When a trade name is used herein, reference to the trade name also refers to the product formulation, the generic drug, and the active pharmaceutical ingredient(s) of the trade name product, unless otherwise indicated by context.

The terms “DR5 binding agent” and “anti-DR5 binding agent” as used herein refers to a molecule, e.g., protein, that specifically binds to DR5. Examples can include a full length anti-DR5 antibody, a fragment of a full length anti-DR5 antibody, or other agent that includes an antibody heavy and/or light chain variable region, and derivatives thereof.

The terms “inhibit” or “inhibition of” as used herein means to reduce by a measurable amount, or to prevent entirely.

The term “deplete,” in the context of the effect of a DR5 binding agent on DR5-expressing cells, refers to a reduction in the number of or elimination of the DR5-expressing cells.

The term “compound” refers to and encompasses the chemical compound itself as well as, whether explicitly stated or not, and unless the context makes clear that the following are to be excluded: amorphous and crystalline forms of the compound, including polymorphic forms, where these forms may be part of a mixture or in isolation; free acid and free base foams of the compound, which are typically the forms shown in the structures provided herein; isomers of the compound, which refers to optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, preferably pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and/or solvates, however, this reference is for emphasis only, and is not to be construed as excluding other of the above Boar's as identified above.

Unless otherwise noted, the term “alkyl” refers to a saturated straight or branched hydrocarbon having from about 1 to about 20 carbon atoms (and all combinations and subcombinations of ranges and specific numbers of carbon atoms therein), with from about 1 to about 8 carbon atoms being preferred. Examples of alkyl groups are methyl, ethyl, n-propyl, iso-propyl, n-butyl, iso-butyl, sec-butyl, tert-butyl, n-pentyl, 2-pentyl, 3-pentyl, 2-methyl-2-butyl, n-hexyl, n-heptyl, n-octyl, n-nonyl, n-decyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl.

Alkyl groups, whether alone or as part of another group, may be referred to as “substituted.” A substituted alkyl group is an alkyl group that is substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl, and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more groups including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the terms “alkenyl” and “alkynyl” refer to straight and branched carbon chains having from about 2 to about 20 carbon atoms (and all combinations and subcombinations of ranges and specific numbers of carbon atoms therein), with from about 2 to about 8 carbon atoms being preferred. An alkenyl chain has at least one double bond in the chain and an alkynyl chain has at least one triple bond in the chain. Examples of alkenyl groups include, but are not limited to, ethylene or vinyl, allyl, -1-butenyl, -2-butenyl, -isobutylenyl, -1-pentenyl, -2-pentenyl, -3-methyl-1-butenyl, -2-methyl-2-butenyl, and -2,3-dimethyl-2-butenyl. Examples of alkynyl groups include, but are not limited to, acetylenic, propargyl, acetylenyl, propynyl, -1-butynyl, -2-butynyl, -1-pentynyl, -2-pentynyl, and—3-methyl-1 butynyl.

As with alkyl groups, alkenyl and alkynyl groups, can be substituted. A “substituted” alkenyl or alkynyl group is one that is substituted with one or more groups, preferably 1 to 3 groups (and any additional substituents selected from halogen), including but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkyenl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be optionally further substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkylene” refers to a saturated branched or straight chain hydrocarbon radical having from about 1 to about 20 carbon atoms (and all combinations and subcombinations of ranges and specific numbers of carbon atoms therein), with from about 1 to about 8 carbon atoms being preferred and having two monovalent radical centers derived by the removal of two hydrogen atoms from the same or two different carbon atoms of a parent alkane. Typical alkylenes include, but are not limited to, methylene, ethylene, propylene, butylene, pentylene, hexylene, heptylene, ocytylene, nonylene, decalene, 1,4-cyclohexylene, and the like. Alkylene groups, whether alone or as part of another group, can be optionally substituted with one or more groups, preferably 1 to 3 groups (and any additional substituents selected from halogen), including, but not limited to, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), -aryl, —C₁-C₈ alkyl, —C₂-C₈ alkenyl, and —C₂-C₈ alkynyl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the term “alkenylene” refers to an optionally substituted alkylene group containing at least one carbon-carbon double bond. Exemplary alkenylene groups include, for example, ethenylene (—CH═CH—) and propenylene (—CH═CHCH₂—).

Unless otherwise noted, the term “alkynylene” refers to an optionally substituted alkylene group containing at least one carbon-carbon triple bond. Exemplary alkynylene groups include, for example, acetylene (—C≡C—), propargyl (—CH₂C≡C—), and 4-pentynyl (—CH₂CH₂CH₂C≡CH—).

Unless otherwise noted, the term “aryl” refers to a monovalent aromatic hydrocarbon radical of 6-20 carbon atoms (and all combinations and subcombinations of ranges and specific numbers of carbon atoms therein) derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, phenyl, naphthalene, anthracene, biphenyl, and the like.

An aryl group, whether alone or as part of another group, can be optionally substituted with one or more, preferably 1 to 5, or even 1 to 2 groups including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, —NO₂, —N₃, —NH₂, —NH(R′), —N(R′)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂—C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Unless otherwise noted, the teem “arylene” refers to an optionally substituted aryl group which is divalent (i.e., derived by the removal of two hydrogen atoms from the same or two different carbon atoms of a parent aromatic ring system) and can be in the ortho, meta, or para configurations as shown in the following structures with phenyl as the exemplary aryl group:

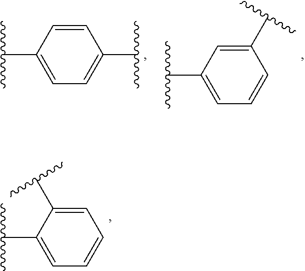

Typical “—(C₁-C₈ alkylene)aryl,” “—(C₂-C₈ alkenylene)aryl”, “and —(C₂-C₈ alkynylene)aryl” groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

Unless otherwise noted, the term “heterocycle,” refers to a monocyclic, bicyclic, or polycyclic ring system having from 3 to 14 ring atoms (also referred to as ring members) wherein at least one ring atom in at least one ring is a heteroatom selected from N, O, P, or S (and all combinations and subcombinations of ranges and specific numbers of carbon atoms and heteroatoms therein). The heterocycle can have from 1 to 4 ring heteroatoms independently selected from N, O, P, or S. One or more N, C, or S atoms in a heterocycle can be oxidized. A monocylic heterocycle preferably has 3 to 7 ring members (e.g., 2 to 6 carbon atoms and 1 to 3 heteroatoms independently selected from N, O, P, or S), and a bicyclic heterocycle preferably has 5 to 10 ring members (e.g., 4 to 9 carbon atoms and 1 to 3 heteroatoms independently selected from N, O, P, or S). The ring that includes the heteroatom can be aromatic or non-aromatic. Unless otherwise noted, the heterocycle is attached to its pendant group at any heteroatom or carbon atom that results in a stable structure.

Heterocycles are described in Paquette, “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. 82:5566 (1960).

Unless otherwise noted, the term “heterocyclo” refers to an optionally substituted heterocycle group as defined above that is divalent (i.e., derived by the removal of two hydrogen atoms from the same or two different carbon atoms of a parent heterocyclic ring system).

Examples of “heterocycle” groups include by way of example and not limitation pyridyl, dihydropyridyl, tetrahydropyridyl (piperidyl), thiazolyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4H-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and isatinoyl. Preferred “heterocycle” groups include, but are not limited to, benzofuranyl, benzothiophenyl, indolyl, benzopyrazolyl, coumarinyl, isoquinolinyl, pyrrolyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl and tetrazolyl.

A heterocycle group, whether alone or as part of another group, can be optionally substituted with one or more groups, preferably 1 to 2 groups, including but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R)₂, —NHC(O)R′, —SR′, —SO₃R′, —S(O)₂R′, —S(O)R′, —OH, —N₃, —NH₂, —NH(R′), —N(R¹)₂ and —CN, where each R′ is independently selected from —H, —C₁-C₈ alkyl, —C₂-C₈ alkenyl, —C₂-C₈ alkynyl, or -aryl and wherein said —O—(C₁-C₈ alkyl), —O—(C₂-C₈ alkenyl), —O—(C₂-C₈ alkynyl), —C₁-C₈ alkyl, —C₂-C₈ alkenyl, —C₂-C₈ alkynyl, and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or aryl.

By way of example and not limitation, carbon-bonded heterocycles can be bonded at the following positions: position 2, 3, 4, 5, or 6 of a pyridine; position 3, 4, 5, or 6 of a pyridazine; position 2, 4, 5, or 6 of a pyrimidine; position 2, 3, 5, or 6 of a pyrazine; position 2, 3, 4, or 5 of a furan, tetrahydrofuran, thiofuran, thiophene, pyrrole or tetrahydropyrrole; position 2, 4, or 5 of an oxazole, imidazole or thiazole; position 3, 4, or 5 of an isoxazole, pyrazole, or isothiazole; position 2 or 3 of an aziridine; position 2, 3, or 4 of an azetidine; position 2, 3, 4, 5, 6, 7, or 8 of a quinoline; or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles can be bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, or 1H-indazole; position 2 of a isoindole, or isoindoline; position 4 of a morpholine; and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

Unless otherwise noted, the term “carbocycle,” refers to a saturated or unsaturated non-aromatic monocyclic, bicyclic, or polycyclic ring system having from 3 to 14 ring atoms (and all combinations and subcombinations of ranges and specific numbers of carbon atoms therein) wherein all of the ring atoms are carbon atoms. Monocyclic carbocycles preferably have 3 to 6 ring atoms, still more preferably 5 or 6 ring atoms. Bicyclic carbocycles preferably have 7 to 12 ring atoms, e.g., arranged as a bicyclo [4,5], [5,5], [5,6] or [6,6] system, or 9 or 10 ring atoms arranged as a bicyclo [5,6] or [6,6] system. The term “carbocycle” includes, for example, a monocyclic carbocycle ring fused to an aryl ring (e.g., a monocyclic carbocycle ring fused to a benzene ring). Carbocyles preferably have 3 to 8 carbon ring atoms.

Carbocycle groups, whether alone or as part of another group, can be optionally substituted with, for example, one or more groups, preferably 1 or 2 groups (and any additional substituents selected from halogen), including, but not limited to, -halogen, —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -aryl, —C(O)R′, —OC(O)R′, —C(O)OR′, —C(O)NH₂, —C(O)NHR′, —C(O)N(R′)₂, —NHC(O)R′, —SR′, —SO₃R′, —S(O)₂R′, —S(O)R′, —OH, ═O, —N₃, —NH₂, —NH(R′), —N(R)₂ and —CN, where each R′ is independently selected from —H, —C₁-C₈ alkyl, —C₂-C₈ alkenyl, —C₂-C₈ alkynyl, or -aryl and wherein said —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), and -aryl groups can be further optionally substituted with one or more substituents including, but not limited to, —C₁-C₈ alkyl, —C₂-C₈ alkenyl, —C₂-C₈ alkynyl, -halogen, —O—(C₁-C₈ alkyl), —O—(C₂-C₈ alkenyl), —O—(C₂-C₈ alkynyl), -aryl, —C(O)R″, —OC(O)R″, —C(O)OR″, —C(O)NH₂, —C(O)NHR″, —C(O)N(R″)₂, —NHC(O)R″, —SR″, —SO₃R″, —S(O)₂R″, —S(O)R″, —OH, —N₃, —NH₂, —NH(R″), —N(R″)₂ and —CN, where each R″ is independently selected from —H, —C₁-C₈ alkyl, —C₂-C₈ alkenyl, —C₂-C₈ alkynyl, or -aryl.

Examples of monocyclic carbocylic substituents include -cyclopropyl, -cyclobutyl, -cyclopentyl, -1-cyclopent-1-enyl, -1-cyclopent-2-enyl, -1-cyclopent-3-enyl, cyclohexyl, -1-cyclohex-1-enyl, -1-cyclohex-2-enyl, -1-cyclohex-3-enyl, -cycloheptyl, -cyclooctyl. -1,3-cyclohexadienyl, -1,4-cyclohexadienyl, -1,3-cycloheptadienyl, -1,3,5-cycloheptatrienyl, and -cyclooctadienyl.

A “carbocyclo,” whether used alone or as part of another group, refers to an optionally substituted carbocycle group as defined above that is divalent (i.e., derived by the removal of two hydrogen atoms from the same or two different carbon atoms of a parent carbocyclic ring system).

Unless otherwise indicated by context, a hyphen (-) designates the point of attachment to the pendant molecule. Accordingly, the term “—(C₁-C₈ alkylene)aryl” or “—C₁-C₈ alkylene(aryl)” refers to a C₁-C₈ alkylene radical as defined herein wherein the alkylene radical is attached to the pendant molecule at any of the carbon atoms of the alkylene radical and one of the hydrogen atoms bonded to a carbon atom of the alkylene radical is replaced with an aryl radical as defined herein.

When a particular group is “substituted”, that group may have one or more substituents, preferably from one to five substituents, more preferably from one to three substituents, most preferably from one to two substituents, independently selected from the list of substituents. The group can, however, generally have any number of substituents selected from halogen. Groups that are substituted are so indicated.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Protective groups as used herein refer to groups which selectively block, either temporarily or permanently, one reactive site in a multifunctional compound. Suitable hydroxy-protecting groups for use in the present invention are pharmaceutically acceptable and may or may not need to be cleaved from the parent compound after administration to a subject in order for the compound to be active. Cleavage is through normal metabolic processes within the body. Hydroxy protecting groups are well known in the art, see, PROTECTIVE GROUPS IN ORGANIC SYNTHESIS by T. W. Greene and P. G. M. Wuts (John Wiley & sons, 3^{rd} Edition) incorporated herein by reference in its entirety and for all purposes and include, for example, ether (e.g., alkyl ethers and silyl ethers including, for example, dialkylsilylether, trialkylsilylether, dialkylalkoxysilylether), ester, carbonate, carbamates, sulfonate, and phosphate protecting groups. Examples of hydroxy protecting groups include, but are not limited to, methyl ether; methoxymethyl ether, methylthiomethyl ether, (phenyldimethylsilyl)methoxymethyl ether, benzyloxymethyl ether, p-methoxybenzyloxymethyl ether, p-nitrobenzyloxymethyl ether, o-nitrobenzyloxymethyl ether, (4-methoxyphenoxy)methyl ether, guaiacolmethyl ether, t-butoxymethyl ether, 4-pentenyloxymethyl ether, siloxymethyl ether, 2-methoxyethoxymethyl ether, 2,2,2-trichloroethoxymethyl ether, bis(2-chloroethoxy)methyl ether, 2-(trimethylsilyl)ethoxymethyl ether, menthoxymethyl ether, tetrahydropyranyl ether, 1-methoxycylcohexyl ether, 4-methoxytetrahydrothiopyranyl ether, 4-methoxytetrahydrothiopyranyl ether S,S-Dioxide, 1-[(2-choro-4-methyl)phenyl]-4-methoxypiperidin-4-yl ether, 1-(2-fluorophenyl)-4-methoxypiperidin-4-yl ether, 1,4-dioxan-2-yl ether, tetrahydrofuranyl ether, tetrahydrothiofuranyl ether; substituted ethyl ethers such as 1-ethoxyethyl ether, 1-(2-chloroethoxy)ethyl ether, 1-[2-(trimethylsilyl)ethoxy]ethyl ether, 1-methyl-1-methoxyethyl ether, 1-methyl-1-benzyloxyethyl ether, 1-methyl-1-benzyloxy-2-fluoroethyl ether, 1-methyl-1phenoxyethyl ether, 2-trimethylsilyl ether, t-butyl ether, allyl ether, propargyl ethers, p-chlorophenyl ether, p-methoxyphenyl ether, benzyl ether, p-methoxybenzyl ether 3,4-dimethoxybenzyl ether, trimethylsilyl ether, triethylsilyl ether, tripropylsilylether, dimethylisopropylsilyl ether, diethylisopropylsilyl ether, dimethylhexylsilyl ether, t-butyldimethylsilyl ether, diphenylmethylsilyl ether, benzoylformate ester, acetate ester, chloroacetate ester, dichloroacetate ester, trichloroacetate ester, trifluoroacetate ester, methoxyacetate ester, triphenylmethoxyacetate ester, phenylacetate ester, benzoate ester, alkyl methyl carbonate, alkyl 9-fluorenylmethyl carbonate, alkyl ethyl carbonate, alkyl 2,2,2,-trichloroethyl carbonate, 1,1,-dimethyl-2,2,2-trichloroethyl carbonate, alkylsulfonate, methanesulfonate, benzylsulfonate, tosylate, methylene acetal, ethylidene acetal, and t-butylmethylidene ketal. Preferred protecting groups are represented by the formulas —R, —Si(R)(R)(R), —C(O)R, —C(O)OR, —C(O)NH(R), —S(O)₂R, —S(O)₂OH, P(O)(OH)₂, and —P(O)(OH)OR, wherein R is C₁-C₂₀ alkyl, C₂-C₂₀ alkenyl, C₂-C₂₀ alkynyl, —C₁-C₂₀ alkylene(carbocycle), —C₂-C₂₀ alkenylene(carbocycle), —C₂-C₂₀ alkynylene(carbocycle), —C₆-C₁₀ aryl, —C₁-C₂₀ alkylene(aryl), —C₂-C₂₀ alkenylene(aryl), —C₂-C₂₀ alkynylene(aryl), —C₁-C₂₀ alkylene(heterocycle), —C₂-C₂₀ alkenylene(heterocycle), or —C₂-C₂₀ alkynylene(heterocycle) wherein said alkyl, alkenyl, alkynyl, alkylene, alkenylene, alkynylene, aryl, carbocycle, and heterocycle radicals whether alone or as part of another group are optionally substituted.

The abbreviation “AFP” refers to dimethylvaline-valine-dolaisoleuine-dolaproine-phenylalanine-p-phenylenediamine (see Formula XVIII infra).

The abbreviation “MMAE” refers to monomethyl auristatin E (see Formula XIII infra).

The abbreviation “AEB” refers to an ester produced by reacting auristatin E with paraacetyl benzoic acid (see Formula XXII infra)

The abbreviation “AEVB” refers to an ester produced by reacting auristatin E with benzoylvaleric acid (see Formula XXIII infra).

The abbreviation “MMAF” refers to dovaline-valine-dolaisoleuine-dolaproine-phenylalanine (see Formula XXI infra).

The term “pharmaceutically acceptable” means approved by a regulatory agency of the Federal or a state government or listed in the U.S. Pharmacopeia or other generally recognized pharmacopeia for use in animals, and more particularly in humans. The term “pharmaceutically compatible ingredient” refers to a pharmaceutically acceptable diluent, adjuvant, excipient, or vehicle with which the antibody or antibody derivative is administered.

The term “animal” refers to humans, non-human mammals (e.g., dogs, cats, rabbits, cattle, horses, sheep, goats, swine, deer, and the like) and non-mammals (e.g., birds, and the like).

### General

The methods described herein encompass the use of Ligand Drug Conjugates wherein the Ligand unit is an anti-DR5 binding agent that specifically binds to DR5. The DR5 binding agent can be, for example, an anti-DR5 antibody, an anti-DR5 antigen-binding fragment, or other DR5 binding agent comprising the amino acid sequence of a humanized antibody heavy and/or light chain variable region, or derivative thereof.

### Ligand Drug Conjugate

The present invention provides, inter alia, Ligand Drug Conjugates for targeted delivery of drugs. The inventors have made the discovery that the Ligand Drug Conjugates have potent cytotoxic and/or cytostatic activity against cells expressing DR5. The Ligand Drug Conjugates comprise a Ligand unit covalently linked to at least one Drug unit. The Drug units can be covalently linked directly or via a Linker unit (-LU-).

In some embodiments, the Ligand Drug Conjugate has the following formula:

L-(LU-D)ₚ (I)

or a pharmaceutically acceptable salt thereof; wherein:
L is the Ligand unit, i.e., a DR5 binding agent of the present invention, and
(LU-D) is a Linker unit-Drug unit moiety, wherein:
LU- is a Linker unit, and
-D is a drug unit having cytostatic or cytotoxic activity against a target cell; and
p is from 1 to 20.

In some embodiments, p ranges from 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is 1, 2, 3, 4, 5 or 6.

In some embodiments, the Ligand Drug Conjugate has the following formula:

L-(Aₐ-W_{w}—Y_{y}-D)ₚ (II)

or a pharmaceutically acceptable salt thereof;
wherein:
L is the Ligand unit, i.e. DR5 binding agent; and
-Aₐ-W_{w}—Y_{y}— is a Linker unit (LU), wherein:
-A- is a Stretcher unit,
a is 0 or 1,
each —W— is independently an Amino Acid unit,
w is an integer ranging from 0 to 12,
—Y— is a self-immolative spacer unit,
y is 0, 1 or 2;
-D is a drug unit having cytostatic or cytotoxic activity against the target cell; and
p is from 1 to 20.

In some embodiments, a is 0 or 1, w is 0 or 1, and y is 0, 1 or 2. In some embodiments, a is 0 or 1, w is 0 or 1, and y is 0 or 1. In some embodiments, p ranges from 1 to 10, 1 to 9, 1 to 8, 1 to 7, 1 to 6, 1 to 5, 1 to 4, 1 to 3, or 1 to 2. In some embodiments, p ranges from 2 to 10, 2 to 9, 2 to 8, 2 to 7, 2 to 6, 2 to 5, 2 to 4 or 2 to 3. In other embodiments, p is 1, 2, 3, 4, 5 or 6. In some embodiments, when w is not zero, y is 1 or 2. In some embodiments, when w is 1 to 12, y is 1 or 2. In some embodiments, w is 2 to 12 and y is 1 or 2. In some embodiments, a is 1 and w and y are 0.

In compositions comprising a plurality of Ligand Drug Conjugates, p is the average number of Drug molecules per Ligand, also referred to as the average drug loading. Average drug loading may range from 1 to about 20 drugs (D) per Ligand. In some embodiments when p represents the average drug loading, p is about 1, about 2, about 3, about, 4, about 5 or about 6. The average number of drugs per ligand in preparation of conjugation reactions may be characterized by conventional means such as mass spectroscopy, ELISA assay, and HPLC. The quantitative distribution of Ligand Drug Conjugates in terms of p may also be determined. In some instances, separation, purification, and characterization of homogeneous Ligand Drug Conjugates where p is a certain value from Ligand Drug Conjugates with other drug loadings may be achieved by means such as reverse phase HPLC or electrophoresis. In exemplary embodiments, p is from 2 to about 8.

The generation of Ligand Drug Conjugates can be accomplished by any technique known to the skilled artisan. Briefly, the Ligand Drug Conjugates comprise an DR5 binding agent as the Ligand unit, a drug, and optionally a linker that joins the drug and the binding agent. A number of different reactions are available for covalent attachment of drugs and/or linkers to binding agents. This is often accomplished by reaction of the amino acid residues of the binding agent, e.g., antibody molecule, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody molecule. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates can also be used as coupling agents for covalently attaching drugs to binding agents. Other techniques are known to the skilled artisan and within the scope of the present invention.

In certain embodiments, an intermediate, which is the precursor of the linker, is reacted with the drug under appropriate conditions. In certain embodiments, reactive groups are used on the drug and/or the intermediate. The product of the reaction between the drug and the intermediate, or the derivatized drug, is subsequently reacted with the DR5 binding agent under appropriate conditions.

Each of the particular units of the Ligand Drug Conjugates is described in more detail herein. The synthesis and structure of exemplary linker units, stretcher units, amino acid units, self-immolative spacer unit, and drug units are also described in U.S. Patent Application Publication Nos. 2003-0083263, 2005-0238649, 2005-0009751, 2006-0074008, and 2009-0010945 each of which is incorporated herein by reference in its entirety and for all purposes.

### Linker Units

Typically, the Ligand Drug Conjugates comprise a linker region between the drug unit and the Ligand unit. In some embodiments, the linker is cleavable under intracellular conditions, such that cleavage of the linker releases the drug unit from the ligand in the intracellular environment. In yet other embodiments, the linker unit is not cleavable and the drug is released, for example, by antibody degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in DR5-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker (SEQ ID NO: ______)). Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the val-cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-5-acetylthioacetate), SPDP(N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker unit is not cleavable and the drug is released by antibody degradation. (See for example U.S. Publication No. 20050238649 incorporated by reference herein in its entirety and for all purposes).

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of Ligand Drug Conjugate, are cleaved when the Ligand Drug Conjugate presents in an extracellular environment (e.g., in plasma). Whether a linker is not substantially sensitive to the extracellular environment can be determined, for example, by incubating with plasma the Ligand Drug Conjugate for a predetermined time period (e.g., 2, 4, 8, 16, or 24 hours) and then quantitating the amount of free drug present in the plasma.

In other, non-mutually exclusive embodiments, the linker promotes cellular internalization. In certain embodiments, the linker promotes cellular internalization when conjugated to the therapeutic agent (i.e., in the milieu of the linker-therapeutic agent moiety of the Ligand Drug Conjugate as described herein). In yet other embodiments, the linker promotes cellular internalization when conjugated to both the auristatin compound and the anti-DR5 antibody.

A variety of exemplary linkers that can be used with the present compositions and methods are described in WO 2004-010957, U.S. Publication No. 20060074008, U.S. Publication No. 20050238649, and U.S. Publication No. 20060024317 (each of which is incorporated by reference herein in its entirety and for all purposes).

A “Linker unit” (LU) is a bifunctional compound that can be used to link a Drug unit and a Ligand unit to form a Ligand Drug Conjugate. In some embodiments, the Linker unit has the formula:

-Aₐ-W_{w}—Y_{y}—

wherein:
-A- is a Stretcher unit,
a is 0 or 1,
each —W— is independently an Amino Acid unit,
w is an integer ranging from 0 to 12,
—Y— is a self-immolative Spacer unit, and
y is 0, 1 or 2.

In some embodiments, a is 0 or 1, w is 0 or 1, and y is 0, 1 or 2. In some embodiments, a is 0 or 1, w is 0 or 1, and y is 0 or 1. In some embodiments, when w is 1 to 12, y is 1 or 2. In some embodiments, w is 2 to 12 and y is 1 or 2. In some embodiments, a is 1 and w and y are 0.

### The Stretcher Unit

The Stretcher unit (A), when present, is capable of linking a Ligand unit to an Amino Acid unit (—W—), if present; to a Spacer unit (—Y—), if present; or to a Drug unit (-D). Useful functional groups that can be present on a DR5 binding agent, either naturally or via chemical manipulation include, but are not limited to, sulfhydryl, amino, hydroxyl, the anomeric hydroxyl group of a carbohydrate, and carboxyl. Suitable functional groups are sulfhydryl and amino. In one example, sulfhydryl groups can be generated by reduction of the intramolecular disulfide bonds of an anti-DR5 antibody. In another embodiment, sulfhydryl groups can be generated by reaction of an amino group of a lysine moiety of an anti-DR5 antibody with 2-iminothiolane (Traut's reagent) or other sulfhydryl generating reagents. In certain embodiments, the anti-DR5 antibody is a recombinant antibody and is engineered to carry one or more lysines. In certain other embodiments, the recombinant anti-DR5 antibody is engineered to carry additional sulfhydryl groups, e.g., additional cysteines.

In one embodiment, the Stretcher unit forms a bond with a sulfur atom of the Ligand unit. The sulfur atom can be derived from a sulfhydryl group of a Ligand. Representative Stretcher units of this embodiment are depicted within the square brackets of Formulas IIIa and IIIb, wherein L-, —W—, —Y—, -D, w and y are as defined above, and R^{a} is selected from —C₁-C₁₀ alkylene-, —C₂-C₁₀ alkenylene-, —C₂-C₁₀ alkynylene-, -carbocyclo-, —O—(C₁-C₈ alkylene)-, O—(C₂-C₈ alkenylene)-, —O—(C₂-C₈ alkynylene)-, -arylene-, —C₁-C₁₀ alkylene-arylene-, —C₂-C₁₀ alkenylene-arylene, —C₂-C₁₀ alkynylene-arylene, -arylene-C₁-C₁₀ alkylene-, -arylene-C₂-C₁₀ alkenylene-, -arylene-C₂-C₁₀ alkynylene-, —C₁-C₁₀ alkylene-(carbocyclo)-, —C₂-C₁₀ alkenylene-(carbocyclo)-, —C₂-C₁₀ alkynylene-(carbocyclo)-, -(carbocyclo)-C₁-C₁₀ alkylene-, -(carbocyclo)-C₂-C₁₀ alkenylene-, -(carbocyclo)-C₂-C₁₀ alkynylene, heterocyclo-, —C₁-C₁₀ alkylene-(heterocyclo)-, —C₂-C₁₀ alkenylene-(heterocyclo)-, —C₂-C₁₀ alkynylene-(heterocyclo)-, -(heterocyclo)-C₁-C₁₀ alkylene-, -(heterocyclo)-C₂-C₁₀ alkenylene-, -(heterocyclo)-C₂-C₁₀ alkynylene-, —(CH₂CH₂O)ᵣ—, or —(CH₂CH₂O)ᵣ—CH₂—, and r is an integer ranging from 1-10, wherein said alkyl, alkenyl, alkynyl, alkylene, alkenylene, alkynylene, aryl, carbocyle, carbocyclo, heterocyclo, and arylene radicals, whether alone or as part of another group, are optionally substituted. In some embodiments, said alkyl, alkenyl, alkynyl, alkylene, alkenylene, alkynylene, aryl, carbocyle, carbocyclo, heterocyclo, and arylene radicals, whether alone or as part of another group, are unsubstituted. In some embodiments, R^{a} is selected from —C₁-C₁₀ alkylene-, -carbocyclo-, —O—(C₁-C₈ alkylene)-, -arylene-, —C₁-C₁₀ alkylene-arylene-, -arylene-C₁-C₁₀ alkylene-, —C₁-C₁₀ alkylene-(carbocyclo)-, -(carbocyclo)-C₁-C₁₀ alkylene-, —C₃-C₈ heterocyclo-, —C₁-C₁₀ alkylene-(heterocyclo)-, -(heterocyclo)-C₁-C₁₀ alkylene-, —(CH₂CH₂O)ᵣ—, and —(CH₂CH₂O), CH₂—; and r is an integer ranging from 1-10, wherein said alkylene groups are unsubstituted and the remainder of the groups are optionally substituted.

It is to be understood from all the exemplary embodiments that even where not denoted expressly, from 1 to 20 drug moieties can be linked to a Ligand (p=1-20).

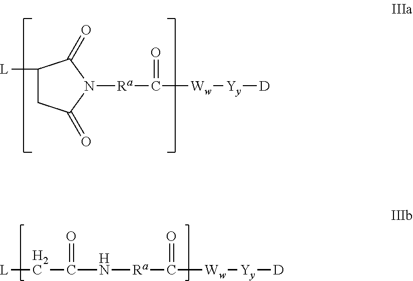

An illustrative Stretcher unit is that of Formula IIIc wherein R^{a} is —(CH₂)₅—:

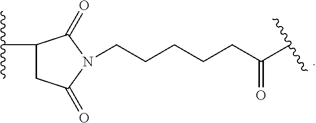

Another illustrative Stretcher unit is that of Formula IIIa wherein R^{a} is —(CH₂CH₂O)ᵣ—CH₂—; and r is 2:

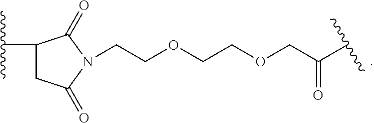

An illustrative Stretcher unit is that of Formula IIIa wherein R^{a} is -arylene- or arylene-C₁-C₁₀ alkylene-. In some embodiments, the aryl group is an unsubstituted phenyl group.

Still another illustrative Stretcher unit is that of Formula IIIb wherein R^{a} is —(CH₂)₅—:

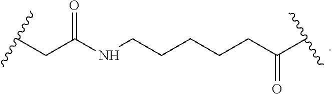

In certain embodiments, the Stretcher unit is linked to the Ligand unit via a disulfide bond between a sulfur atom of the Ligand unit and a sulfur atom of the Stretcher unit. A representative Stretcher unit of this embodiment is depicted within the square brackets of Formula IV, wherein R^{a}, L-, —W—, —Y—, -D, w and y are as defined above.

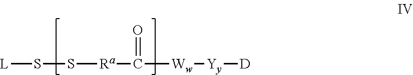

It should be noted that throughout this application, the S moiety in the formula below refers to a sulfur atom of the Ligand unit, unless otherwise indicated by context.

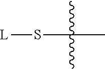

In yet other embodiments, the Stretcher, prior to attachment to L, contains a reactive site that can form a bond with a primary or secondary amino group of the Ligand. Examples of these reactive sites include, but are not limited to, activated esters such as succinimide esters, 4 nitrophenyl esters, pentafluorophenyl esters, tetrafluorophenyl esters, anhydrides, acid chlorides, sulfonyl chlorides, isocyanates and isothiocyanates. Representative Stretcher units of this embodiment are depicted within the square brackets of Formulas Va and Vb, wherein -R^{a}-, L-, —W—, —Y—, -D, w and y are as defined above;

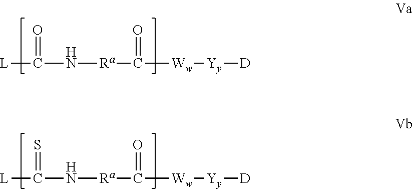

In some embodiments, the Stretcher contains a reactive site that is reactive to a modified carbohydrate's (—CHO) group that can be present on a Ligand. For example, a carbohydrate can be mildly oxidized using a reagent such as sodium periodate and the resulting (—CHO) unit of the oxidized carbohydrate can be condensed with a Stretcher that contains a functionality such as a hydrazide, an oxime, a primary or secondary amine, a hydrazine, a thiosemicarbazone, a hydrazine carboxylate, and an arylhydrazide such as those described by Kaneko et al., 1991, Bioconjugate Chem. 2:133-41. Representative Stretcher units of this embodiment are depicted within the square brackets of Formulas VIa, VIb, and VIc, wherein -R^{a}-, L-, —W—, —Y—, -D, w and y are as defined as above.

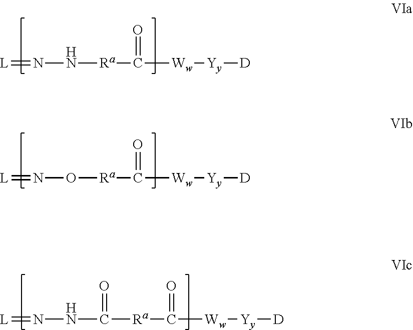

### The Amino Acid Unit

The Amino Acid unit (—W—), when present, links the Stretcher unit to the Spacer unit if the Spacer unit is present, links the Stretcher unit to the Drug moiety if the Spacer unit is absent, and links the Ligand unit to the Drug unit if the Stretcher unit and Spacer unit are absent.

W_{w}— can be, for example, a monopeptide, dipeptide, tripeptide, tetrapeptide, pentapeptide, hexapeptide, heptapeptide, octapeptide, nonapeptide, decapeptide, undecapeptide or dodecapeptide unit. Each —W— unit independently has the formula denoted below in the square brackets, and w is an integer ranging from 0 to 12:

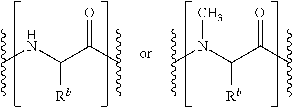

wherein R^{b} is hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl,

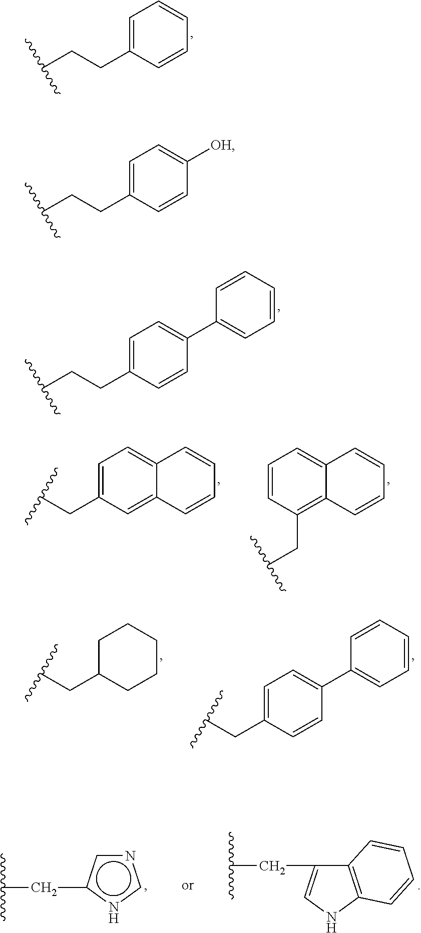

In some embodiments, the Amino Acid unit can be enzymatically cleaved by one or more enzymes, including a cancer or tumor-associated protease, to liberate the Drug unit (-D), which in one embodiment is protonated in vivo upon release to provide a Drug (D).

In certain embodiments, the Amino Acid unit can comprise natural amino acids. In other embodiments, the Amino Acid unit can comprise non-natural amino acids. Illustrative W_{w} units are represented by formulas (VII)-(IX):

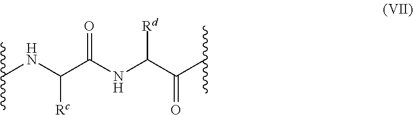

wherein R^{c} and R^{d} are as follows:

| R^{c} | R^{d} |
|---|---|
| Benzyl | (CH₂)₄NH₂; |
| methyl | (CH₂)₄NH₂; |
| isopropyl | (CH₂)₄NH₂; |
| isopropyl | (CH₂)₃NHCONH₂; |
| benzyl | (CH₂)₃NHCONH₂; |
| isobutyl | (CH₂)₃NHCONH₂; |
| sec-butyl | (CH₂)₃NHCONH₂; |
| 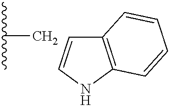 | (CH₂)₃NHCONH₂; |
| benzyl | methyl; |
| benzyl | (CH₂)₃NHC(═NH)NH₂; |

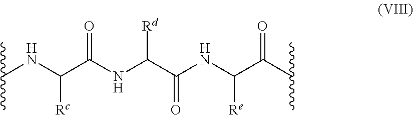

wherein R^{c}, R^{d} and R^{e} are as follows:

| R^{c} | R^{d} | R^{e} |
|---|---|---|
| benzyl | Benzyl | (CH₂)₄NH₂; |
| isopropyl | Benzyl | (CH₂)₄NH₂; and |
| H | Benzyl | (CH₂)₄NH₂; |

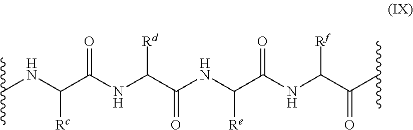

wherein R^{c}, R^{d}, R^{e} and R^{f} are as follows:

| R^{c} | R^{d} | R^{e} | R^{f} |
|---|---|---|---|
| H | benzyl | isobutyl | H; and |
| methyl | isobutyl | methyl | isobutyl. |

Exemplary Amino Acid units include, but are not limited to, units of formula VII where: R^{c} is benzyl and R^{d} is —(CH₂)₄NH₂; R^{c} is isopropyl and R^{d} is —(CH₂)₄NH₂; or R^{c} is isopropyl and R^{d} is —(CH₂)₃NHCONH₂. Another exemplary Amino Acid unit is a unit of formula VIII wherein R^{c} is benzyl, R^{d} is benzyl, and R^{e} is —(CH₂)₄NH₂.

Useful —W_{w}— units can be designed and optimized in their selectivity for enzymatic cleavage by a particular enzyme, for example, a tumor-associated protease. In one embodiment, a —W_{w}— unit is that whose cleavage is catalyzed by cathepsin B, C and D, or a plasmin protease.

In one embodiment, —W_{w}— is a dipeptide, tripeptide, tetrapeptide or pentapeptide. When R^{b}, R^{c}, R^{d}, R^{e} or R^{f} is other than hydrogen, the carbon atom to which R^{b}, R^{c}, R^{d}, R^{e} or R^{f} is attached is chiral.

Each carbon atom to which R^{b}, R^{c}, R^{d}, R^{e} or R^{f} is attached is independently in the (S) or (R) configuration.

In one aspect of the Amino Acid unit, the Amino Acid unit is valine-citrulline (vc or val-cit). In another aspect, the Amino Acid unit is phenylalanine-lysine (i.e., fk). In yet another aspect of the Amino Acid unit, the Amino Acid unit is N-methylvaline-citrulline. In yet another aspect, the Amino Acid unit is 5-aminovaleric acid, homo phenylalanine lysine, tetraisoquinolinecarboxylate lysine, cyclohexylalanine lysine, isonepecotic acid lysine, beta-alanine lysine, glycine serine valine glutamine and isonepecotic acid.

### The Spacer Unit

The Spacer unit (—Y—), when present, links an Amino Acid unit to the Drug unit when an Amino Acid unit is present. Alternately, the Spacer unit links the Stretcher unit to the Drug unit when the Amino Acid unit is absent. The Spacer unit also links the Drug unit to the Ligand unit when both the Amino Acid unit and Stretcher unit are absent.

Spacer units are of two general types: non self-immolative or self-immolative. A non self-immolative Spacer unit is one in which part or all of the Spacer unit remains bound to the Drug moiety after cleavage, particularly enzymatic, of an Amino Acid unit from the ligand-drug conjugate. Examples of a non self-immolative Spacer unit include, but are not limited to a (glycine-glycine) Spacer unit and a glycine Spacer unit (both depicted in Scheme 1) (infra). When a conjugate containing a glycine-glycine Spacer unit or a glycine Spacer unit undergoes enzymatic cleavage via an enzyme (e.g., a tumor-cell associated-protease, a cancer-cell-associated protease or a lymphocyte-associated protease), a glycine-glycine-Drug moiety or a glycine-Drug moiety is cleaved from L-Aa-Ww-. In one embodiment, an independent hydrolysis reaction takes place within the target cell, cleaving the glycine-Drug moiety bond and liberating the Drug.

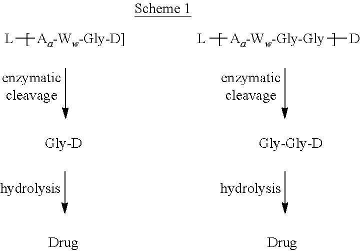

In some embodiments, a non self-immolative Spacer unit (—Y—) is -Gly-. In some embodiments, a non self-immolative Spacer unit (—Y—) is -Gly-Gly-.

In one embodiment, a Drug-Linker conjugate is provided in which the Spacer unit is absent (y=0), or a pharmaceutically acceptable salt thereof.

Alternatively, a conjugate containing a self-immolative Spacer unit can release -D. As used herein, the term “self-immolative Spacer” refers to a bifunctional chemical moiety that is capable of covalently linking together two spaced chemical moieties into a stable tripartite molecule. It will spontaneously separate from the second chemical moiety if its bond to the first moiety is cleaved.

In some embodiments, —Y_{y}— is a p-aminobenzyl alcohol (PAB) unit (see Schemes 2 and 3) whose phenylene portion is substituted with Qₘ wherein Q is —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -halogen, -nitro or -cyano; and m is an integer ranging from 0-4. The alkyl, alkenyl and alkynyl groups, whether alone or as part of another group, can be optionally substituted.

In some embodiments, —Y— is a PAB group that is linked to —W_{w}— via the amino nitrogen atom of the PAB group, and connected directly to -D via a carbonate, carbamate or ether group. Without being bound by any particular theory or mechanism, Scheme 2 depicts a possible mechanism of Drug release of a PAB group which is attached directly to -D via a carbamate or carbonate group as described by Toki et al., 2002, J. Org. Chem. 67:1866-1872.

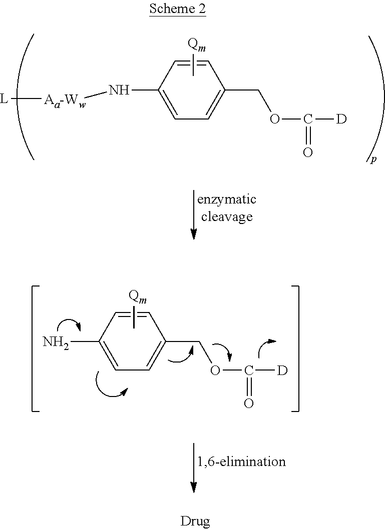

In Scheme 2, Q is —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Without being bound by any particular theory or mechanism, Scheme 3 depicts a possible mechanism of Drug release of a PAB group which is attached directly to -D via an ether or amine linkage, wherein D includes the oxygen or nitrogen group that is part of the Drug unit.

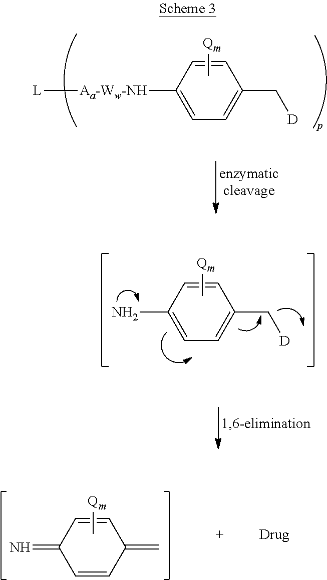

In Scheme 3, Q is —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -halogen, -nitro or -cyano; m is an integer ranging from 0-4; and p ranges from 1 to about 20. The alkyl, alkenyl and alkynyl groups, whether alone or as part of another group, can be optionally substituted.

Other examples of self-immolative spacers include, but are not limited to, aromatic compounds that are electronically similar to the PAB group such as 2-aminoimidazol-5-methanol derivatives (Hay et al., 1999, Bioorg. Med. Chem. Lett. 9:2237) and ortho or para-aminobenzylacetals. Spacers can be used that undergo cyclization upon amide bond hydrolysis, such as substituted and unsubstituted 4-aminobutyric acid amides (Rodrigues et al., 1995, Chemistry Biology 2:223), appropriately substituted bicyclo[2.2.1] and bicyclo[2.2.2] ring systems (Storm et al., 1972, J. Amer. Chem. Soc. 94:5815) and 2-aminophenylpropionic acid amides (Amsberry et al., 1990, J. Org. Chem. 55:5867). Elimination of amine-containing drugs that are substituted at the α-position of glycine (Kingsbury et al., 1984, J. Med. Chem. 27:1447) are also examples of self-immolative spacers.

In one embodiment, the Spacer unit is a branched bis(hydroxymethyl)-styrene (BHMS) unit as depicted in Scheme 4, which can be used to incorporate and release multiple drugs.

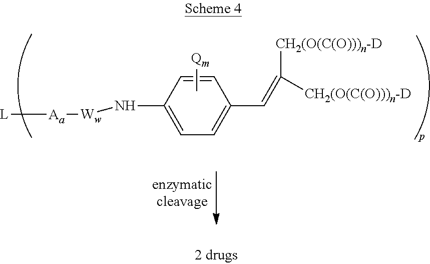

In Scheme 4, Q is —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -halogen, -nitro or -cyano; m is an integer ranging from 0-4; n is 0 or 1; and p is an integer of from 1 to about 20. The alkyl, alkenyl and alkynyl groups, whether alone or as part of another group, can be optionally substituted.

In some embodiments, the -D moieties are the same. In yet another embodiment, the -D moieties are different.

In one aspect, Spacer units (—Y_{y}—) are represented by Formulae (X)-(XII):

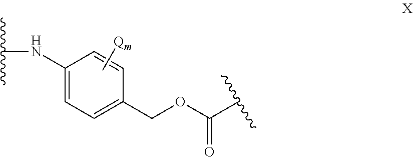

wherein Q is —C₁-C₈ alkyl, —C₂-C₈ alkenyl, —C₂-C₈ alkynyl, —O—(C₁-C₈ alkyl), —O—(C₂-C₈ alkenyl), —O—(C₂-C₈ alkynyl), -halogen, -nitro or -cyano; and m is an integer ranging from 0-4. The alkyl, alkenyl and alkynyl groups, whether alone or as part of another group, can be optionally substituted.

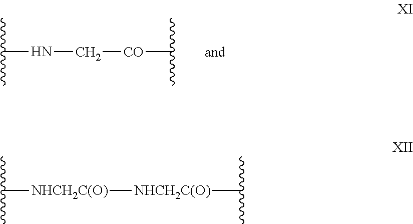

In a group of selected embodiments, the conjugates of Formula I and II are:

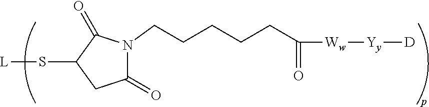

wherein w and y are each 0, 1 or 2,
and,

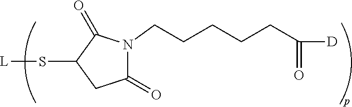

wherein w and y are each 0,

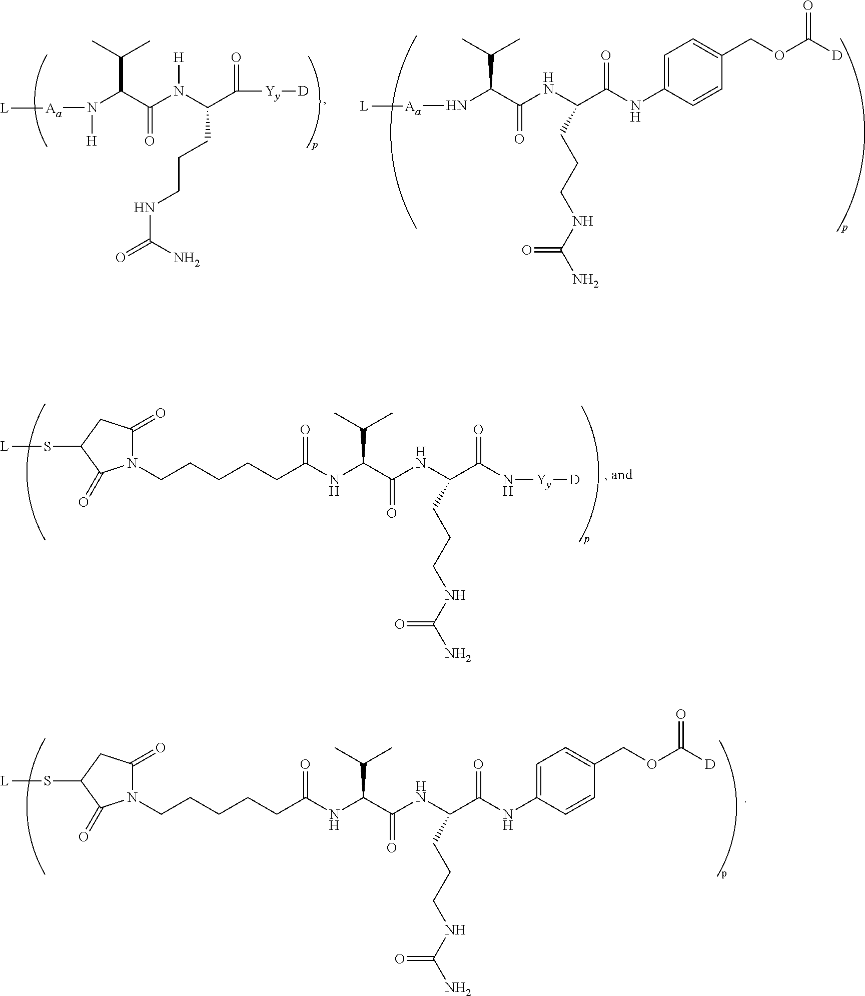

wherein Aₐ, W_{W}, Y_{y}, D and L have the meanings provided above.

### The Drug Unit

The drug moiety (D) can be any cytotoxic, cytostatic or immunomodulatory (e.g., immunosuppressive) agent or drug. D is a Drug unit (moiety) having an atom that can form a bond with the Spacer unit, with the Amino Acid unit, with the Stretcher unit or with the Ligand unit. In some embodiments, the Drug unit D has a nitrogen atom that can form a bond with the Spacer unit. As used herein, the terms “drug unit” and “drug moiety” are synonymous and used interchangeably.

Useful classes of cytotoxic or immunomodulatory agents include, for example, antitubulin agents, DNA minor groove binders, DNA replication inhibitors, and alkylating agents.

In some embodiments, the Drug is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication Nos. 2003-0083263, 2005-0238649 and 2005-0009751; International Patent Publication No. WO 04/010957, International Patent Publication No. WO 02/088172, and U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins of the present invention bind tubulin and can exert a cytotoxic or cytostatic effect on a DR5 expressing cell line. There are a number of different assays, known in the art, that can be used for determining whether an auristatin or resultant antibody-drug conjugate exerts a cytostatic or cytotoxic effect on a desired cell line.

Methods for determining whether a compound binds tubulin are known in the art. See, for example, Muller et al., Anal. Chem. 2006, 78, 4390-4397; Hamel et al., Molecular Pharmacology, 1995 47: 965-976; and Hamel et al., The Journal of Biological Chemistry, 1990 265:28, 17141-17149. For purposes of the present invention, the relative affinity of a compound to tubulin can be determined. Some preferred auristatins of the present invention bind tubulin with an affinity ranging from 10 fold lower (weaker affinity) than the binding affinity of MMAE to tubulin to 10 fold, 20 fold or even 100 fold higher (higher affinity) than the binding affinity of MMAE to tubulin.

In some embodiments, -D is an auristatin of the formula D_{E} or D_{F}:

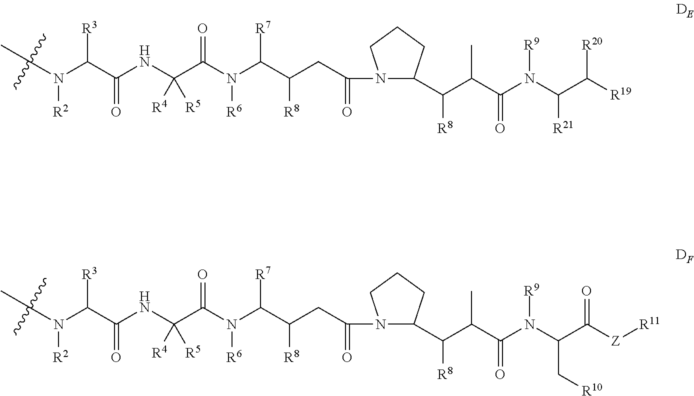

or a pharmaceutically acceptable salt form thereof; wherein, independently at each location: the wavy line indicates a bond;
R² is —C₁-C₂₀ alkyl, —C₂-C₂₀ alkenyl, or —C₂-C₂₀ alkynyl;
R³ is —H, —C₁-C₂₀ alkyl, —C₂-C₂₀ alkenyl, —C₂-C₂₀ alkynyl, carbocycle, —C₁-C₂₀ alkylene (carbocycle), —C₂-C₂₀ alkenylene(carbocycle), —C₂-C₂₀ alkynylene(carbocycle), -aryl, —C₁-C₂₀ alkylene(aryl), —C₂-C₂₀ alkenylene(aryl), —C₂-C₂₀ alkynylene(aryl), -heterocycle, —C₁-C₂₀ alkylene(heterocycle), —C₂-C₂₀ alkenylene(heterocycle), or —C₂-C₂₀ alkynylene(heterocycle);
R⁴ is —H, —C₁-C₂₀ alkyl, —C₂-C₂₀ alkenyl, —C₂-C₂₀ alkynyl, carbocycle, —C₁-C₂₀ alkylene (carbocycle), —C₂-C₂₀ alkenylene(carbocycle), —C₂-C₂₀ alkynylene(carbocycle), -aryl, —C₁-C₂₀ alkylene(aryl), —C₂-C₂₀ alkenylene(aryl), —C₂-C₂₀ alkynylene(aryl), -heterocycle, —C₁-C₂₀ alkylene(heterocycle), —C₂-C₂₀ alkenylene(heterocycle), or —C₂-C₂₀ alkynylene(heterocycle);
R⁵ is —H or —C₁-C₈ alkyl;
or R⁴ and R⁵ jointly form a carbocyclic ring and have the formula —(CR^{a}R^{b})ₛ— wherein R^{a} and R^{b} are independently —H, —C₁-C₂₀ alkyl, —C₂-C₂₀ alkenyl, —C₂-C₂₀ alkynyl, or -carbocycle and s is 2, 3, 4, 5 or 6,
R⁶ is —H, —C₁-C₂₀ alkyl, —C₂-C₂₀ alkenyl, or —C₂-C₂₀ alkynyl;
R⁷ is —H, —C₁-C₂₀ alkyl, —C₂-C₂₀ alkenyl, —C₂-C₂₀ alkynyl, -carbocycle, —C₁-C₂₀ alkylene (carbocycle), —C₂-C₂₀ alkenylene(carbocycle), —C₂-C₂₀ alkynylene(carbocycle), -aryl, —C₁-C₂₀ alkylene(aryl), —C₂-C₂₀ alkenylene(aryl), —C₂-C₂₀ alkynylene(aryl), heterocycle, —C₁-C₂₀ alkylene(heterocycle), —C₂-C₂₀ alkenylene(heterocycle), or —C₂-C₂₀ alkynylene(heterocycle);
each R⁸ is independently —H, —OH, —C₁-C₂₀ alkyl, —C₂-C₂₀ alkenyl, —C₂-C₂₀ alkynyl, —O—(C₁-C₂₀ alkyl), —O—(C₂-C₂₀ alkenyl), —O—(C₁-C₂₀ alkynyl), or -carbocycle;
R⁹ is —H, —C₁-C₂₀ alkyl, —C₂-C₂₀ alkenyl, or —C₂-C₂₀ alkynyl;
R¹⁹ is -aryl, -heterocycle, or -carbocycle;
R²⁰ is —H, —C₁-C₂₀ alkyl, —C₂-C₂₀ alkenyl, —C₂-C₂₀ alkynyl, -carbocycle, —O—(C₁-C₂₀ alkyl), —O—(C₂-C₂₀ alkenyl), —O—(C₂-C₂₀ alkynyl), or OR¹⁸ wherein R¹⁸ is —H, a hydroxyl protecting group, or a direct bond where OR¹⁸ represents ═O;
R²¹ is —H, —C₁-C₂₀ alkyl, —C₂-C₂₀ alkenyl, or —C₂-C₂₀ alkynyl, -aryl, heterocycle, or -carbocycle;
R¹⁰ is -aryl or -heterocycle;
Z is —O—, —S—, —NH—, or —NR¹²—, wherein R¹² is —C₁-C₂₀ alkyl, —C₂-C₂₀ alkenyl, or —C₂-C₂₀ alkynyl;
R¹¹ is —H, —C₁-C₂₀ alkyl, —C₂-C₂₀ alkenyl, —C₂-C₂₀ alkynyl, -aryl, -heterocycle, —(R¹³O)ₘ—R¹⁴, or —(R¹³O)ₘ—CH(R¹⁵)₂;
m is an integer ranging from 1-1000;
R¹³ is —C₂-C₂₀ alkylene, —C₂-C₂₀ alkenylene, or —C₂-C₂₀ alkynylene;
R¹⁴ is —H, —C alkyl, alkenyl, or —C₂-C₂₀ alkynyl;
each occurrence of R¹⁵ is independently —H, —COOH, —(CH₂)ₙ—N(R¹⁶)₂, —(CH₂)ₙ—SO₃H, —(CH₂)ₙ—SO₃—C₁-C₂₀ alkyl, —(CH₂)ₙ—SO₃—C₂-C₂₀ alkenyl, or —(CH₂)ₙ—SO₃—C₂-C₂₀ alkynyl; each occurrence of R¹⁶ is independently —H, —C₁-C₂₀ alkyl, —C₂-C₂₀ alkenyl, —C₂-C₂₀ alkynyl or —(CH₂)ₙ—COOH; and
n is an integer ranging from 0 to 6; wherein said alkyl, alkenyl, alkynyl, alkylene, alkenylene, alkynyklene, aryl, carbocyle, and heterocycle radicals, whether alone or as part of another group, are optionally substituted.

Auristatins of the formula D_{E} include those wherein said alkyl, alkenyl, alkynyl, alkylene, alkenylene, alkynyklene, aryl, carbocyle, and heterocycle radicals are unsubstituted.

Auristatins of the formula D_{E} include those wherein the groups of R², R³, R⁴, R⁵, R⁶, R⁷, R⁸, and R⁹ are unsubstituted and the groups of R¹⁹, R²⁰ and R²¹ are optionally substituted as described herein.

Auristatins of the formula D_{E} include those wherein

R² is —C₁-C₈ alkyl;

R³, R⁴ and R⁷ are independently selected from —H, —C₁-C₂₀ alkyl, —C₂-C₂₀ alkenyl, —C₂-C₂₀ alkynyl, monocyclic C₃-C₆ carbocycle, —C₁-C₂₀ alkylene(monocyclic C₃-C₆ carbocycle), —C₂-C₂₀ alkenylene(monocyclic C₃-C₆ carbocycle), —C₂-C₂₀ alkynylene(monocyclic C₃-C₆ carbocycle), —C₆-C₁₀ aryl, —C₁-C₂₀ alkylene(C₆-C₁₀ aryl), —C₂-C₂₀ alkenylene(C₆-C₁₀ aryl), —C₂-C₂₀ alkynylene(C₆-C₁₀ aryl), -heterocycle, —C₁-C₂₀ alkylene(heterocycle), —C₂-C₂₀ alkenylene(heterocycle), or —C₂-C₂₀ alkynylene(heterocycle); wherein said alkyl, alkenyl, alkynyl, alkylene, alkenylene, alkynylene, carbocycle, aryl, and heterocycle radicals are optionally substituted;

R⁵ is -hydrogen;

R⁶ is —C₁-C₈ alkyl;

each R⁸ is independently selected from —OH, —O—(C₁-C₂₀ alkyl), —O—(C₂-C₂₀ alkenyl), or —O—(C₂-C₂₀ alkynyl) wherein said alkyl, alkenyl, and alkynyl radicals are optionally substituted;

R⁹ is -hydrogen or —C₁-C₈ alkyl;

R¹⁹ is optionally substituted phenyl;

R²⁰ is OR¹⁸; wherein R¹⁸ is H, a hydroxyl protecting group, or a direct bond where OR¹⁸ represents ═O;

R²¹ is selected from —H, —C₁-C₂₀ alkyl, —C₂-C₂₀ alkenyl, —C₂-C₂₀ alkynyl, or -carbocycle; wherein said alkyl, alkenyl, alkynyl, and carbocycle radicals are optionally substituted; or a pharmaceutically acceptable salt form thereof.

Auristatins of the formula D_{E} include those wherein

R² is methyl;

R³ is —H, —C₁-C₈ alkyl, —C₂-C₈ alkenyl, or —C₂-C₈ alkynyl, wherein said alkyl, alkenyl and alkynyl radicals are optionally optionally substituted;

R⁴ is —H, —C₁-C₈ alkyl, —C₂-C₈ alkenyl, —C₂-C₈ alkynyl, monocyclic C₃-C₆ carbocycle, —C₆-C₁₀ aryl, —C₁-C₈ alkylene(C₆-C₁₀ aryl), —C₂-C₈ alkenylene(C₆-C₁₀ aryl), —C₂-C₈ alkynylene(C₆-C₁₀ aryl), —C₁-C₈ alkylene (monocyclic C₃-C₆ carbocycle), —C₂-C₈ alkenylene (monocyclic C₃-C₆ carbocycle), —C₂-C₈ alkynylene(monocyclic C₃-C₆ carbocycle); wherein said alkyl, alkenyl, alkynyl, alkylene, alkenylene, alkynylene, aryl, and carbocycle radicals whether alone or as part of another group are optionally substituted;

R⁵ is H; R⁶ is methyl;

R⁷ is —C₁-C₈ alkyl, —C₂-C₈ alkenyl or —C₂-C₈ alkynyl;

each R⁸ is methoxy;

R⁹ is -hydrogen or —C₁-C₈ alkyl;

R¹⁹ is phenyl;

R²⁰ is OR¹⁸; wherein R¹⁸ is —H, a hydroxyl protecting group, or a direct bond where OR¹⁸ represents ═O;

R²¹ is methyl; or a pharmaceutically acceptable salt form thereof.

Auristatins of the formula D_{E} include those wherein

R² is methyl; R³ is H or C₁-C₃ alkyl; R⁴ is C₁-C₅ alkyl; R⁵ is H; R⁶ is methyl; R⁷ is isopropyl or sec-butyl; R⁸ is methoxy; R⁹ is hydrogen or C₁-C₈ alkyl; R¹⁹ is phenyl; R²⁰ is OR¹⁸; wherein R¹⁸ is H, a hydroxyl protecting group, or a direct bond where OR¹⁸ represents ═O; and R²¹ is methyl; or a pharmaceutically acceptable salt form thereof.

Auristatins of the formula D_{E} include those wherein

R² is methyl or or C₁-C₃ alkyl; R³ is H or C₁-C₃ alkyl; R⁴ is C₁-C₅ alkyl; R⁵ is H; R⁶ is C₁-C₃ alkyl; R⁷ is C₁-C₅ alkyl; R⁸ is C₁-C₃ alkoxy; R⁹ is hydrogen or C₁-C₈ alkyl; R¹⁹ is phenyl; R²⁰ is OR¹⁸; wherein R¹⁸ is H, a hydroxyl protecting group, or a direct bond where OR¹⁸ represents ═O; and R²¹ is C₁-C₃ alkyl; or a pharmaceutically acceptable salt form thereof.

Auristatins of the formula D_{F} include those wherein

R² is methyl;

R³, R⁴, and R⁷ are independently selected from —H, —C₁-C₂₀ alkyl, —C₂-C₂₀ alkenyl, —C₂-C₂₀ alkynyl, monocyclic C₃-C₆ carbocycle, —C₁-C₂₀ alkylene(monocyclic C₃-C₆ carbocycle), —C₂-C₂₀ alkenylene(monocyclic C₃-C₆ carbocycle), —C₂-C₂₀ alkynylene(monocyclic C₃-C₆ carbocycle), —C₆-C₁₀ aryl, —C₁-C₂₀ alkylene(C₆-C₁₀ aryl), —C₂-C₂₀ alkenylene(C₆-C₁₀ aryl), —C₂-C₂₀ alkynylene(C₆-C₁₀ aryl), -heterocycle, —C₁-C₂₀ alkylene(heterocycle), —C₂-C₂₀ alkenylene(heterocycle), or —C₂-C₂₀ alkynylene(heterocycle); wherein said alkyl, alkenyl, alkynyl, alkylene, alkenylene, alkynylene, carbocyle, aryl, and heteocycle radicals whether alone or as part of another group are optionally substituted;

R⁵ is —H;

R⁶ is methyl;

each R⁸ is methoxy;

R⁹ is —H, —C₁-C₂₀ alkyl, —C₂-C₂₀ alkenyl, or —C₂-C₂₀ alkynyl; wherein said alkyl, alkenyl and alkynyl radical are optionally substituted;

R¹⁰ is optionally substituted aryl or optionally substituted heterocycle;

Z is —O—, —S—, —NH—, or —NR¹²—, wherein R¹² is —C₁-C₂₀ alkyl, —C₂-C₂₀ alkenyl, or —C₂-C₂₀ alkynyl, each of which is optionally substituted;

R¹¹ is —H, —C₁-C₂₀ alkyl, —C₂-C₂₀ alkenyl, —C₂-C₂₀ alkynyl, -aryl, -heterocycle, —(R¹³O)ₘ—R¹⁴, or —(R¹³O)ₘ—CH(R¹⁵)₂ wherein said alkyl, alkenyl, alkyny, aryl, and heterocycle radicals are optionally substituted;

m is an integer ranging from 1-1000;

R¹³ is —C₂-C₂₀ alkylene, —C₂-C₂₀ alkenylene, or —C₂-C₂₀ alkynylene, each of which is optionally substituted;

R¹⁴ is —H, —C₁-C₂₀ alkyl, —C₂-C₂₀ alkenyl, or —C₂-C₂₀ alkynyl wherein said alkyl, alkenyl and alkynyl radicals are optionally substituted;

each occurrence of R¹⁵ is independently —H, —COOH, —(CH₂)ₙ—N(R¹⁶)₂, —(CH₂)ₙ—SO₃H, —(CH₂)ₙ—SO₃—C₁-C₂₀ alkyl, —(CH₂)ₙ—SO₃—C₂-C₂₀ alkenyl, or —(CH₂)ₙ—SO₃—C₂-C₂₀ alkynyl wherein said alkyl, alkenyl and alkynyl radicals are optionally substituted;

each occurrence of R¹⁶ is independently —H, —C₁-C₂₀ alkyl, —C₂-C₂₀ alkenyl, —C₂-C₂₀ alkynyl or —(CH₂)ₙ—COOH wherein said alkyl, alkenyl and alkynyl radicals are optionally substituted;

n is an integer ranging from 0 to 6; or a pharmaceutically acceptable salt form thereof.

In certain of these embodiments, R¹⁰ is optionally substituted phenyl;

Auristatins of the formula D_{F} include those wherein the groups of R², R³, R⁴, R⁵, R⁶, R⁷, R⁸, and R⁹ are unsubstituted and the groups of R¹⁰ and R¹¹ are as described herein.

Auristatins of the formula D_{F} include those wherein said alkyl, alkenyl, alkynyl, alkylene, alkenylene, alkynyklene, aryl, carbocyle, and heterocycle radicals are unsubstituted.

Auristatins of the formula D_{F} include those wherein

R² is C₁-C₃ alkyl; R³ is H or C₁-C₃ alkyl; R⁴ is C₁-C₅ alkyl; R⁵ is H; R⁶ is C₁-C₃ alkyl; R⁷ is C₁-C₅ alkyl; R⁸ is C₁-C₃ alkoxy; R⁹ is hydrogen or C₁-C₈ alkyl;

R¹⁰ is optionally substituted phenyl; Z is O, S, or NH; and R¹¹ is as defined herein; or a pharmaceutically acceptable salt form thereof.

Auristatins of the formula D_{F} include those wherein

R² is methyl; R³ is H or C₁-C₃ alkyl; R⁴ is C₁-C₅ alkyl; R⁵ is H; R⁶ is methyl; R⁷ is isopropyl or sec-butyl; R⁸ is methoxy; R⁹ is hydrogen or C₁-C₈ alkyl;

R¹⁰ is optionally substituted phenyl; Z is O, S, or NH; and R¹¹ is as defined herein; or a pharmaceutically acceptable salt form thereof.

Auristatins of the formula D_{F} include those wherein

R² is methyl; R³ is H or C₁-C₃ alkyl; R⁴ is C₁-C₅ alkyl; R⁵ is H; R⁶ is methyl; R⁷ is isopropyl or sec-butyl; R⁸ is methoxy; R⁹ is hydrogen or C₁-C₈ alkyl; R¹⁰ is phenyl; and Z is O or NH and R¹¹ is as defined herein, preferably hydrogen; or a pharmaceutically acceptable salt form thereof.

Auristatins of the formula D_{F} include those wherein

R² is C₁-C₃ alkyl; R³ is H or C₁-C₃ alkyl; R⁴ is C₁-C₅ alkyl; R⁵ is H; R⁶ is C₁-C₃ alkyl; R⁷ is C₁-C₅ alkyl; R⁸ is C₁-C₃ alkoxy; R⁹ is hydrogen or C₁-C₈ alkyl;

R¹⁰ is phenyl; and Z is O or NH and R¹¹ is as defined herein, preferably hydrogen; or a pharmaceutically acceptable salt form thereof.

Auristatins of the formula D_{E} or D_{F} include those wherein R³, R⁴ and R⁷ are independently isopropyl or sec-butyl and R⁵ is —H. In an exemplary embodiment, R³ and R⁴ are each isopropyl, R⁵ is H, and R⁷ is sec-butyl. The remainder of the substituents are as defined herein.

Auristatins of the formula D_{E} or D_{F} include those wherein R² and R⁶ are each methyl, and R⁹ is H. The remainder of the substituents are as defined herein.

Auristatins of the formula D_{E} or D_{F} include those wherein each occurrence of R⁸ is —OCH₃. The remainder of the substituents are as defined herein.

Auristatins of the formula D_{E} or D_{F} include those wherein R³ and R⁴ are each isopropyl, R² and R⁶ are each methyl, R⁵ is H, R⁷ is sec-butyl, each occurrence of R⁸ is —OCH₃, and R⁹ is H. The remainder of the substituents are as defined herein.

Auristatins of the formula D_{F} include those wherein Z is —O— or —NH—. The remainder of the substituents are as defined herein.

Auristatins of the formula D_{F} include those wherein R¹⁰ is aryl. The remainder of the substituents are as defined herein.

Auristatins of the formula D_{F} include those where R¹⁰ is -phenyl. The remainder of the substituents are as defined herein.

Auristatins of the formula D_{F} include those wherein Z is —O—, and R¹¹ is H, methyl or t-butyl. The remainder of the substituents are as defined herein.

Auristatins of the formula D_{F} include those wherein, when Z is —NH, R¹¹ is —(R¹³O)ₘ—CH(R¹⁵)₂, wherein R¹⁵ is —(CH₂)ₙ—N(R¹⁶)₂, and R¹⁶ is —C₁-C₈ alkyl or —(CH₂)ₙ—COOH. The remainder of the substituents are as defined herein.

Auristatins of the formula D_{F} include those wherein when Z is —NH, R¹¹ is —(R¹³O)ₘ—CH(R¹⁵)₂, wherein R¹⁵ is —(CH₂)ₙ—SO₃H. The remainder of the substituents are as defined herein.

In preferred embodiments, when D is an auristatin of formula D_{E}, w is an integer ranging from 1 to 12, preferably 2 to 12, y is 1 or 2, and a is preferably 1.

In some embodiments, wheren D is an auristatin of formula D_{F}, a is 1 and w and y are 0.

Illustrative Drug units (-D) include the drug units having the following structures:

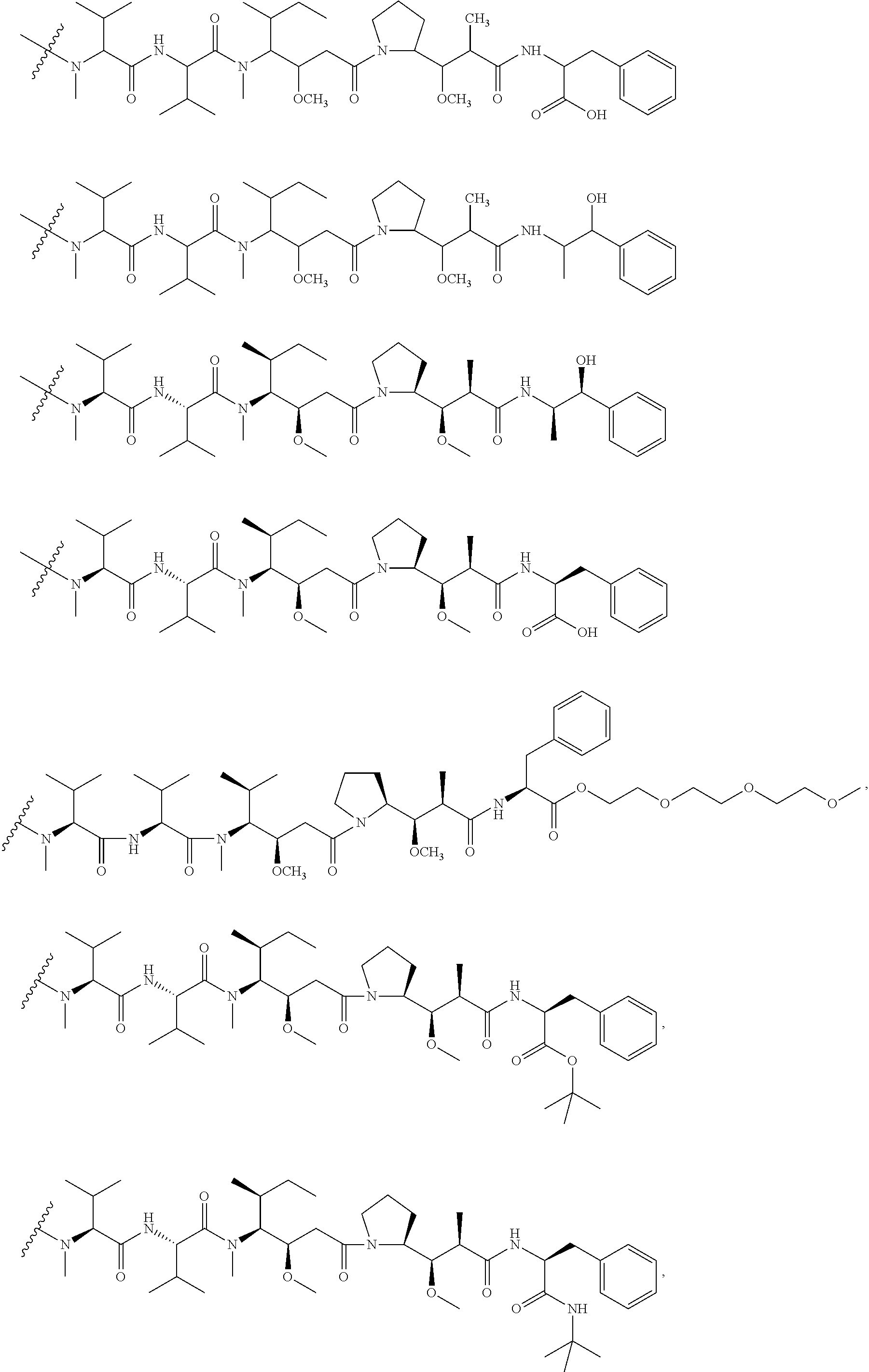

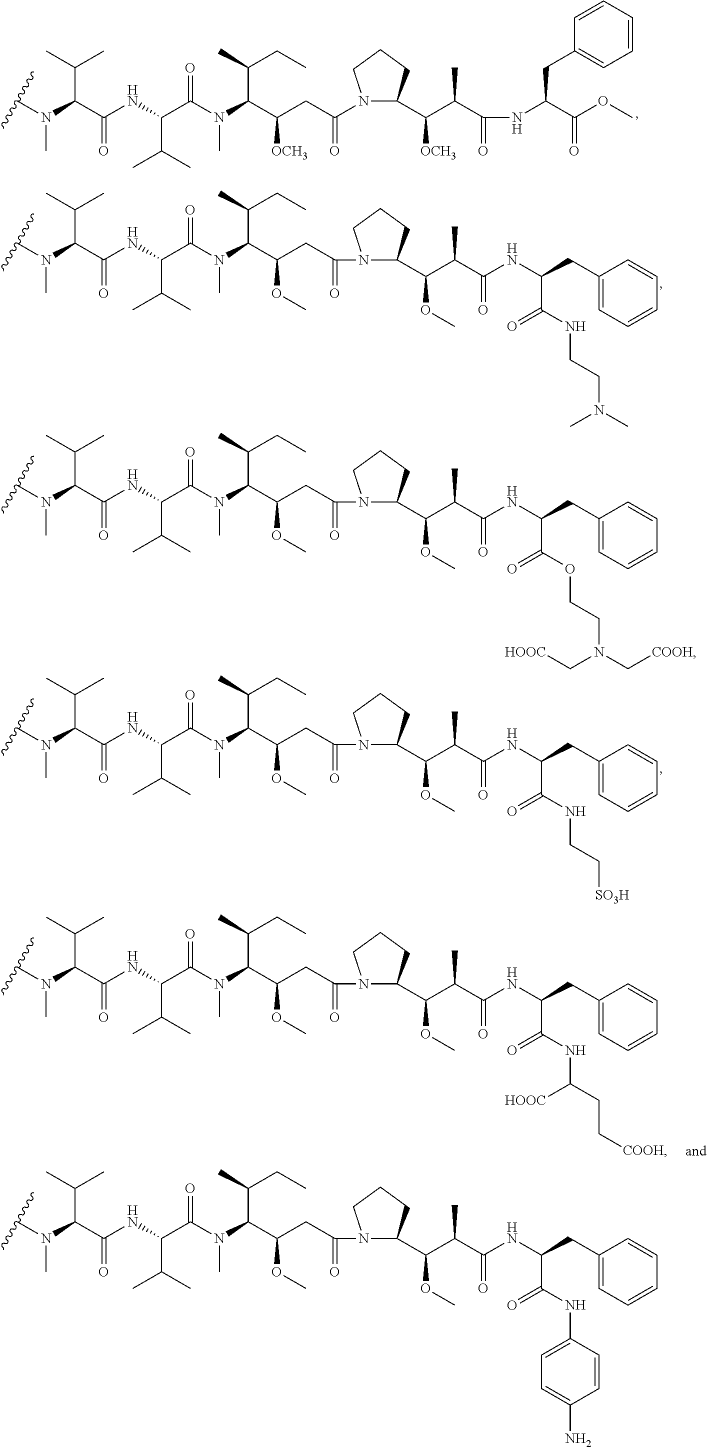

or pharmaceutically acceptable salts or solvates thereof.

In one aspect, hydrophilic groups, such as but not limited to triethylene glycol esters (TEG) can be attached to the Drug Unit at R¹¹. Without being bound by theory, the hydrophilic groups assist in the internalization and non-agglomeration of the Drug unit.

In some embodiments, the Drug unit is not TZT-1027. In some embodiments, the Drug unit is not auristatin E, dolastatin 10, or auristatin PE.

Exemplary Ligand Drug Conjugates have the following structures wherein “mAb” represents an anti-DR⁵ antibody and S is a sulfur atom of the antibody. The subscript p is an integer of from 1 to about 20 and is preferably 1 to about 5.

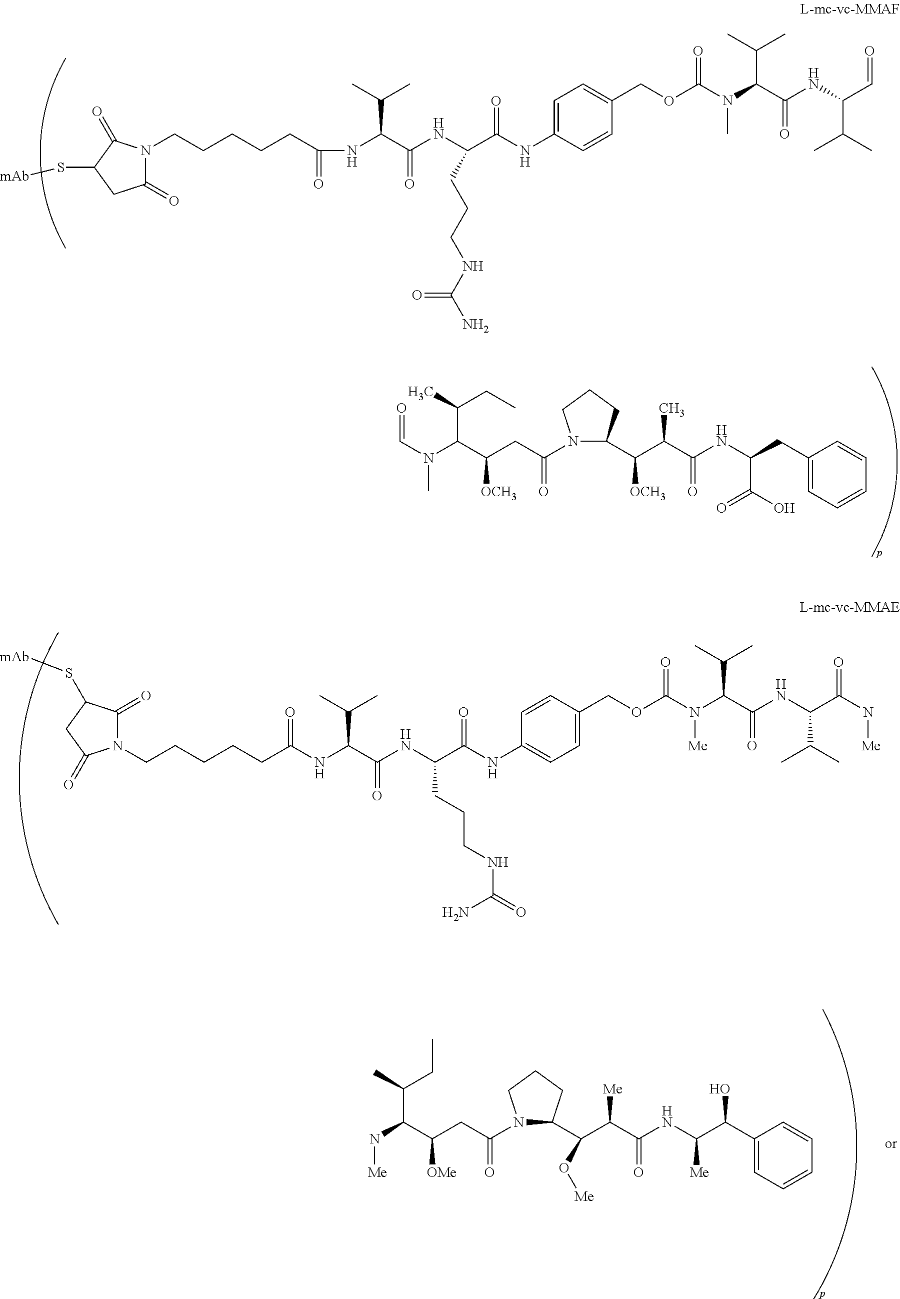

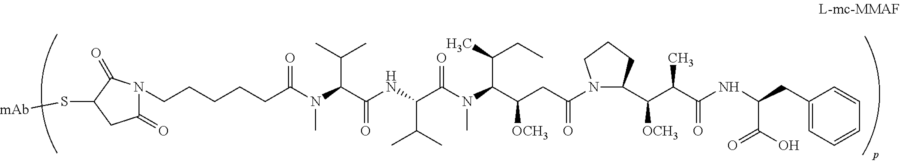

or pharmaceutically acceptable salt forms thereof.

In some embodiments, the Drug Unit is a calicheamicin, camptothecin, a maytansinoid, or an anthracycline. In some embodiments the drug is a taxane, a topoisomerase inhibitor, a vinca alkaloid, or the like.

In some typical embodiments, suitable cytotoxic agents include, for example, DNA minor groove binders (e.g., enediynes and lexitropsins, a CBI compound; see also U.S. Pat. No. 6,130,237), duocarmycins, taxanes (e.g., paclitaxel and docetaxel), puromycins, and vinca alkaloids. Other cytotoxic agents include, for example, CC-1065, SN-38, topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone.

In some embodiments, the Drug is an anti-tubulin agent. Examples of anti-tubulin agents include, auristatins, taxanes (e.g., Taxol® (paclitaxel), Taxotere® (docetaxel)), T67 (Tularik) and vinca alkyloids (e.g., vincristine, vinblastine, vindesine, and vinorelbine). Other antitubulin agents include, for example, baccatin derivatives, taxane analogs (e.g., epothilone A and B), nocodazole, colchicine and colcimid, estramustine, cryptophycins, cemadotin, maytansinoids, combretastatins, discodermolide, and eleutherobin.

In certain embodiments, the cytotoxic agent is a maytansinoid, another group of anti-tubulin agents. For example, in specific embodiments, the maytansinoid is maytansine or DM-1 (ImmunoGen, Inc.; see also Chari et al., 1992, Cancer Res. 52:127-131).

In certain embodiments, the cytotoxic or cytostatic agent is a dolastatin. In certain embodiments, the cytotoxic or cytostatic agent is of the auristatin class. Thus, in a specific embodiment, the cytotoxic or cytostatic agent is MMAE (Formula XIII) In another specific embodiment, the cytotoxic or cytostatic agent is AFP (Formula XVIII).

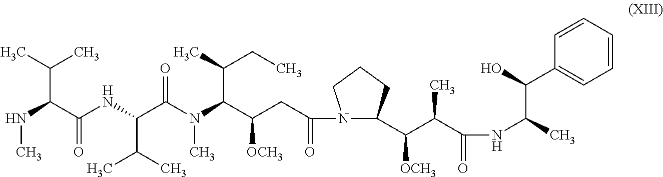

In certain embodiments, the cytotoxic or cytostatic agent is a compound of formulas XII-XXI or pharmaceutically acceptable salt form thereof:

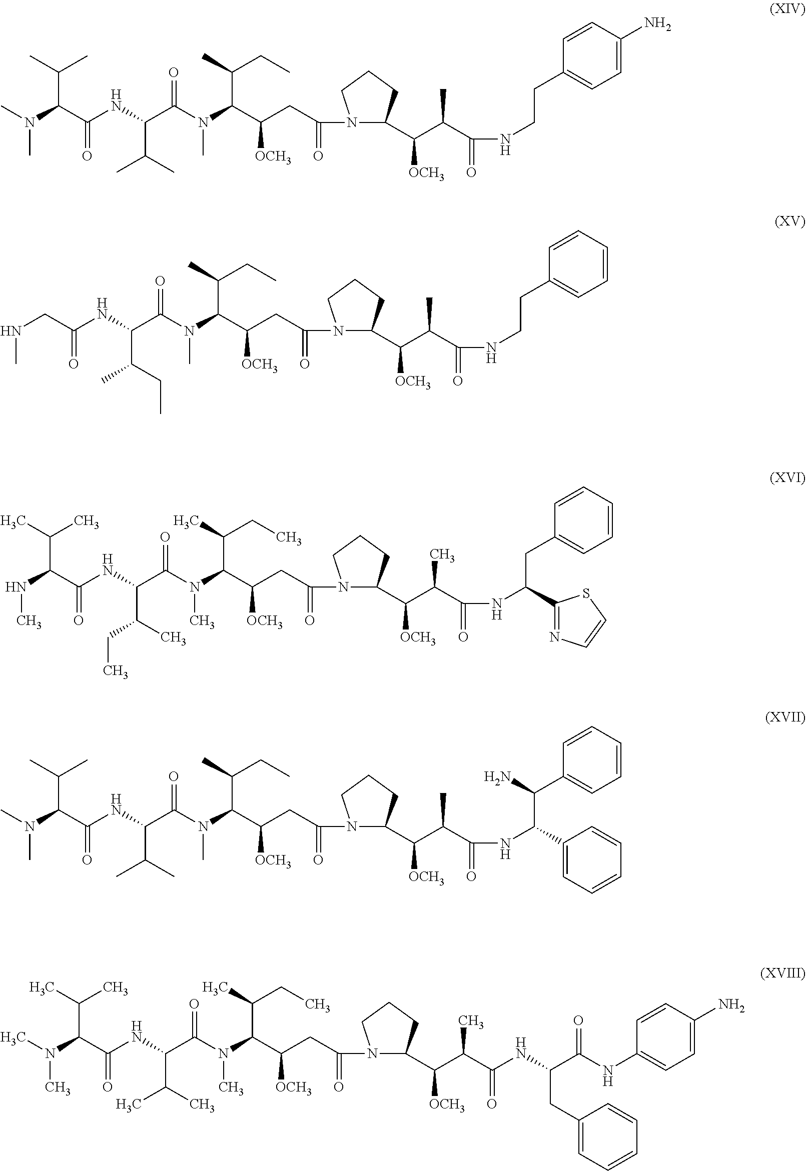

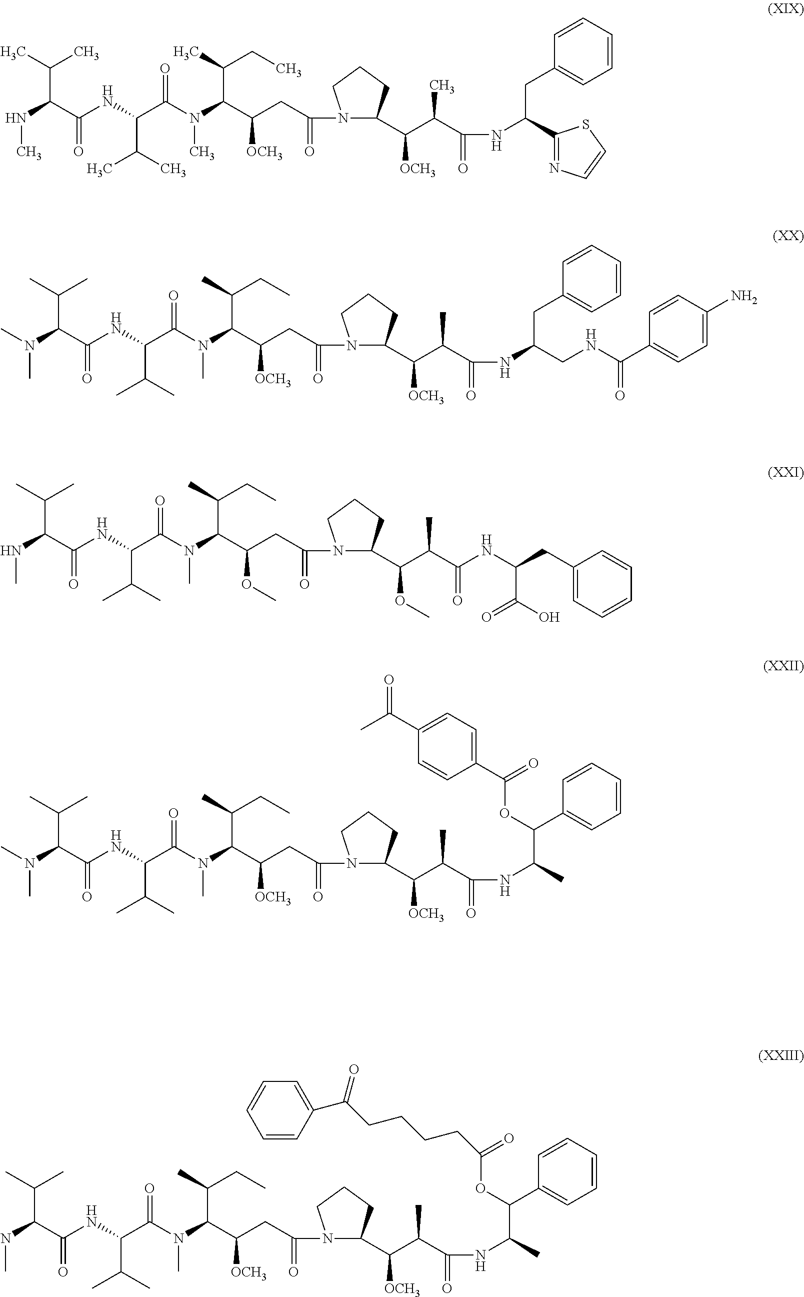

### Ligand Unit

In the present invention, the Ligand unit (e.g., an antibody) in the Ligand Drug Conjugate specifically binds to DR5 and exhibits cytotoxic activity via internalization. The Ligand Drug Conjugate reaches cancer tissue expressing DR5 to which the Ligand unit (e.g., an antibody) specifically binds as its target. As a result, the Drug unit conjugated to the antibody can be allowed to selectively act on the target cells. Therefore, the efficacy of the antibody-drug conjugate can be more greatly enhanced than that of the antibody alone. Antibodies that bind to death domain-containing receptors, especially an anti-DR5 antibody, can be selected as an antibody that can be contained in the antibody-drug conjugate according to the present invention.

Antibodies Binding to DR5

(1) DR5 Gene

The nucleotide sequence and amino acid sequence of the human death receptor 5 (DR5) gene has been registered as GI:22547118 (accession no. NM_{—}147187) in GenBank. A nucleotide sequence coding an amino acid sequence with one or more amino acids replaced, deleted, or added in the amino acid sequence of DR5 and having bioactivity comparable to that of DR5 is also included in the nucleotide sequence of the DR5 gene. In addition, a protein that consists of an amino acid sequence with one or more amino acids replaced, deleted, or added in the amino acid sequence of DR5 and that has bioactivity comparable to that of DR5 is also included in DR5.

(2) Antibody Against DR5

The antibody against DR5 according to the present invention can be obtained in the usual way by immunizing an animal with DR5 or any polypeptide selected from the amino acid sequence of DR5. Such antibody produced in the living body can be collected and purified.

In addition, a monoclonal antibody can also be obtained from a hybridoma established by fusing an antibody-producing cell that produces an antibody against DR5 with a myeloma cell according to a known method (for example, Kohler and Milstein, Nature (1975) 256, p. 495-497; Kennet, R. ed., Monoclonal Antibody, p. 365-367, Prenum Press, N.Y. (1980)).

DR5 as the antigen can be obtained from genetically engineered host cells expressing the DR5 gene.

More specifically, DR5 can be obtained by preparing a vector that can express the DR5 gene, introducing the vector into host cells to express the gene, and purifying the expressed DR5.

In addition, after an artificial gene fusing the extracellular region of DR5 with the constant region of an antibody is constructed, a protein prepared in an appropriate expression system of the gene can also be used as an immunogen.

(3) Other Antibodies

In addition to the monoclonal antibody against the above DR5, the antibodies according to the present invention include recombinant antibodies artificially altered to reduce heterologous antigenicity against humans, such as chimeric antibodies, humanized antibodies, and human antibodies. These antibodies can be produced by means of known methods.

Such chimeric antibodies include an antibody whose variable region and constant region are heterologous to each other, and an example thereof is a chimeric antibody created by joining the variable region genes of a mouse-derived antibody to human constant region genes (Proc. Natl. Acad. Sci. U.S.A., 81, 6851-6855, (1984)).

Examples of such humanized antibodies include an antibody in which only the complementarity-determining regions (CDRs) are transferred into a human antibody (Nature (1986) 321, p. 522-525) and an antibody in which CDR sequences and amino acid residues in part of the framework are grafted into a human antibody by CDR grafting (International Publication No. WO90/07861).

In addition, there are human antibodies. The term human anti DR5 antibody refers to a human antibody that only has gene sequences of a human chromosome-derived antibody. The anti-human DR5 antibody can be obtained by a method that uses a human antibody-producing mouse having a chromosome fragment containing H- and L-chain genes for a human antibody (Tomizuka, K. et al., Nature Genetics (1997) 16, p. 133-143; Kuroiwa, Y. et. al., Nuc. Acids Res. (1998) 26, p. 3447-3448; Yoshida, H. et. al., Animal Cell Technology: Basic and Applied Aspects vol. 10, p. 69-73 (Kitagawa, Y., Matuda, T. and lijima, S. eds.), Kluwer Academic Publishers, 1999; Tomizuka, K. et. al., Proc. Natl. Acad. Sci. USA (2000) 97, p. 722-727).

Such a transgenic animal, or more specifically, a genetically modified animal in which the gene loci for endogenous immunoglobulin heavy and light chains in a nonhuman mammal are destroyed and instead the gene loci for human immunoglobulin heavy and light chains are introduced into this knockout animal via a yeast artificial chromosome (YAC) vector or the like, can be produced by preparing a knockout animal and a transgenic animal as mentioned above and crossbreeding these animals.

The antibody can also be obtained from culture supernatant produced by transforming eukaryotic cells with cDNA, preferably a vector containing the cDNA coding for each of the humanized antibody heavy and light chains by recombinant DNA technology and culturing the transformed cells producing a recombinant human monoclonal antibody.

Here, examples of cells that can be used as a host include eukaryotic cells, preferably mammalian cells such as CHO cells, lymphocytes, and myeloma.

In addition, a method of obtaining a phage display-derived human antibody screened from a human antibody library (Wormstone, I. M. et. al, Investigative Ophthalmology & Visual Science (2002) 43 (7), p. 2301-2308; Carmen, S. et. al., Briefings in Functional Genomics and Proteomics (2002), 1 (2), p. 189-203; Siriwardena, D. et. al., Opthalmology (2002) 109 (3), p. 427-431) is also known.

For example, a human antibody heavy and light variable regions are displayed on a phage surface as a single-chain antibody (scFv) and then an antigen-binding phage is selected (Nature Biotechnology (2005), 23, (9), p. 1105-1116).

The DNA sequence coding an antigen-binding human antibody variable region can be determined by analyzing the genes of the phage selected by antigen binding.

Once the DNA sequence of the antigen-binding scFv becomes clear, a human antibody can be obtained by preparing an expression vector having the sequence and introducing the vector into an appropriate host for expression (WO92/01047, WO92/20791, WO93/06213, WO93/11236, WO93/19172, WO95/01438, WO95/15388; Annu. Rev. Immunol (1994) 12, p. 433-455; Nature Biotechnology (2005) 23 (9), p. 1105-1116).

When the antibody genes are once isolated and then introduced into an appropriate host to prepare the antibody, an appropriate combination of a host and an expression vector can be used.

When eukaryotic cells are used as a host, animal cells, plant cells, or eukaryotic microorganisms can be used.

Examples of such animal cells include simian COS cells (Gluzman, Y., Cell (1981) 23, p. 175-182, ATCC CRL-1650), murine fibroblasts NIH3T3 (ATCC No. CRL-1658), and dihydrofolate reductase-deficient strains of Chinese hamster ovary cells (CHO cells, ATCC CCL-61) (Urlaub, G. and Chasin, L. A., Proc. Natl. Acad. Sci. U.S.A. (1980) 77, p. 4216-4220).

Examples of prokaryotic cells that can be used include Escherichia coli and Bacillus subtilis.

The antibody can be obtained by introducing the antibody genes of interest into these cells by transformation and culturing the transformed cells in vitro.

The isotype of the antibody according to the present invention is not limited, examples thereof include IgG (IgG1, IgG2, IgG3, and IgG4), IgM, IgA (IgA1 and IgA2), IgD, and IgE, but IgG and IgM are preferable.

In addition, the antibody according to the present invention may be a fragment of an antibody having an antigen-binding site of the antibody or a modified version thereof if it maintains antigen binding.

Examples of such antibody functional fragments include Fab, F(ab′)₂, a monovalent variable region fragment Fab′ obtained by reducing F(ab′)₂, Fv, single-chain Fv (scFv) obtained by linking heavy-chain and light-chain Fv by an appropriate linker, diabody (diabodies), linear antibodies, and polyspecific antibodies formed of antibody fragments, but the fragments are not limited to the above fragments if they maintain antigen binding. The above antibody fragments can be obtained by processing full-length antibody molecules with an enzyme such as papain or pepsin. The above antibody fragments can also be obtained by using nucleic acid sequences coding the heavy chain and light chain of the above antibody fragments to allow an appropriate gene expression system to produce the corresponding proteins.

These antibody fragments can be produced by obtaining and expressing the genes in the same way as above to allow a host to produce the corresponding proteins.

The antibody according to the present invention may be a polyclonal antibody, a mixture of several anti-DR5 antibodies having different amino acid sequences. An example of such a polyclonal antibody is a mixture of several antibodies having different CDRs. As such a polyclonal antibody, an antibody obtained by culturing a mixture of cells producing different antibodies and purifying the culture can be used (WO2004/061104).

The antibody obtained can be uniformly purified. The separation and purification of the antibody may be conducted by means of the separation and purification methods used for normal proteins.

For example, the antibody can be separated and purified by appropriately selecting and combining chromatography columns, filters, ultrafiltration, salting-out, dialysis, preparative polyacrylamide gel electrophoresis, isoelectric focusing, and the like (Strategies for Protein Purification and Charcterization: A Laboratoy Course Manual, Daniel R. Marshak et al. Cold Spring Harbor Laboratory Press (1996); Antibodies: A Laboratory Manual. Ed Harlow and David Lane, Cold Spring Harbor Laboratory (1988)) but the separation and purification methods are not limited to these.

Examples of chromatography include affinity chromatography, ion exchange chromatography, hydrophobic chromatography, gel filtration, reversed phase chromatography, and adsorption chromatography. These types of chromatography can be performed by using liquid-phase chromatography such as HPLC and FPLC.

Examples of the columns used in affinity chromatography include protein A columns and protein G columns.

Examples of the protein A columns include Hyper D, POROS, Sepharose F. F. (Pharmacia).

In addition, the antibody can also be purified by its binding to the antigen immobilized on a carrier.

(4) Examples of Anti-DR5 Antibodies

For example, the anti-DR5 antibodies inducing apoptosis in DR5-expressing cells described in International Publication Nos. WO98/51793, WO2001/83560, WO2002/94880, WO2003/54216, WO2004/50895, WO2006/83971, and WO2007/22157 can be used as components of the antibody-drug conjugate according to the present invention. In addition, anti-DR5 antibodies called Lexatumumab, HGS-TR2J, Apomab, Apomab7.3, Conatumumab, and LBY135 and variants thereof can also be used as components of the antibody-drug conjugate according to the present invention. However, the antibodies that can be used as such components are not limited to the above examples if such antibodies have a capacity to bind to DR5 protein.

The Ligand unit of the present invention is typically a DR5 binding agent. In one group of embodiments, the Ligand unit comprises a heavy chain amino acid sequence corresponding to humanized TRA-8 (SEQ ID NO:1). Humanized TRA-8 is abbreviated as hTRA-8 in the specification. In another group of embodiments, Ligand unit comprises a light chain amino acid sequence corresponding to humanized TRA-8 (SEQ ID NO:2). In yet another embodiment, the Ligand unit comprises both a heavy and light chain amino acid sequence of SEQ ID NOs: 1 and 2. The anti-DR5 antibody used as a Ligand unit in this embodiment has an International Nonproprietary Name, Tigatuzumab. In still another embodiment, the Ligand unit comprises (a) a heavy chain immunoglobulin having the CDR1 consisting of amino residues 1-5 of SEQ ID NO:3, the CDR2 consisting of amino acid residues 1-17 of SEQ ID NO:4, and the CDR3 consisting of amino acid residues 1-10 of SEQ ID NO:5; and (b) a light chain immunoglobulin having the CDR1 consisting of amino residues 1-11 of SEQ ID NO: 6, the CDR2 consisting of amino acid residues 1-7 of SEQ ID NO:7, and the CDR3 consisting of amino acid residues 1-8 of SEQ ID NO:8. In another embodiment, the Ligand unit comprises the heavy chain variable region of hTRA-8 comprising amino acid residues 1-118 of SEQ ID NO:1 and the light chain variable region of hTRA-8 comprising amino acid residues 1-107 of SEQ ID NO:2.

Additionally, the Ligand unit (L) has at least one functional group that can form a bond with a functional group of a Linker unit. Useful functional groups that can be present on a Ligand unit, either naturally, via chemical manipulation or via engineering, include, but are not limited to, sulfhydryl (—SH), amino, hydroxyl, carboxy, the anomeric hydroxyl group of a carbohydrate, and carboxyl. In some embodiments, a Ligand unit functional group is a sulfhydryl group. The sulfhydryl group is typically a solvent accessible sulfhydryl group, such as a solvent accessible sulfhydryl group on a cysteine residue. Sulfhydryl groups can be generated by reduction of an intramolecular or inter molecular disulfide bond of a Ligand. Sulfhydryl groups also can be generated by reaction of an amino group of a lysine moiety of a Ligand using 2-iminothiolane (Traut's reagent) or another sulfhydryl generating reagent.

In some embodiments, one or more sulfhydryl groups are engineered into a Ligand unit, such as by amino acid substitution. For example, a sulfhydryl group can be introduced into a Ligand unit. In some embodiments, a sulfhydryl group is introduced by an amino acid substitution of serine or threonine to a cysteine residue, and/or by addition of a cysteine residue into a Ligand unit (an engineered cysteine residue). In some embodiments, the cysteine residue is an internal cysteine residue, i.e., not located at the N-terminus or C-terminus of the Ligand moiety.

In an exemplary embodiment, a cysteine residue can be engineered into an antibody heavy or light variable region (e.g., of an antibody fragment, such as a diabody) by amino acid substitution. The amino acid substitution is typically introduced into the framework region and is located distal to the epitope-binding face of the variable region. For example, the amino acid substitution can be at least 10 angstroms, at least 20 angstroms or at least 25 angstroms from the epitope-binding face or the CDRs. Suitable positions for substitution of a cysteine residue can be determined based on the known or predicted three dimensional structures of antibody variable regions. (See generally Holliger and Hudson, 2005, Nature BioTechnology 23(9):1126-1136.) In exemplary embodiments, a serine to cysteine amino acid substitution is introduced at amino acid position 84 of the V_{H} region and/or position 14 of the V_{L} region (according to the numbering system of Kabat et al., Sequences of Proteins of Immunological Interest, 5th edition, (Bethesda, Md., NIH) 1991).

To control the number of Drug or Linker unit-Drug units attached to a Ligand unit, one or more cysteine residues can be eliminated by amino acid substitution. For example, the number of solvent accessible cysteine residues in an immunoglobulin hinge region can be reduced by amino acid substitution of cysteine to serine residues.

In some embodiments, a Ligand unit contains 1, 2, 3, 4, 5, 6 7 or 8 solvent-accessible cysteine residues. In some embodiments, a Ligand unit contains 2 or 4 solvent-accessible cysteine residues.

### Assay

Methods of determining whether a Drug or Ligand Drug Conjugate exerts a cytostatic and/or cytotoxic effect on a cell are known. Generally, the cytotoxic or cytostatic activity of a Ligand Drug Conjugate can be measured by: exposing mammalian cells expressing a target protein of the Ligand Drug Conjugate in a cell culture medium; culturing the cells for a period from about 6 hours to about 5 days; and measuring cell viability. Cell-based in vitro assays can be used to measure viability (proliferation), cytotoxicity, and induction of apoptosis (caspase activation) of the Ligand Drug Conjugate.

For determining whether a Ligand Drug Conjugate exerts a cytostatic effect, a thymidine incorporation assay may be used. For example, cancer cells expressing a target antigen at a density of 5,000 cells/well of a 96-well plated can be cultured for a 72-hour period and exposed to 0.5 μCi of ³H-thymidine during the final 8 hours of the 72-hour period. The incorporation of ³H-thymidine into cells of the culture is measured in the presence and absence of the Ligand Drug Conjugate.

For determining cytotoxicity, necrosis or apoptosis (programmed cell death) can be measured. Necrosis is typically accompanied by increased permeability of the plasma membrane; swelling of the cell, and rupture of the plasma membrane. Apoptosis is typically characterized by membrane blebbing, condensation of cytoplasm, and the activation of endogenous endonucleases. Determination of any of these effects on cancer cells indicates that a Ligand Drug Conjugate is useful in the treatment of cancers.

Cell viability can be measured by determining in a cell the uptake of a dye such as neutral red, trypan blue, or ALAMAR™ blue (see, e.g., Page et al., 1993, Intl. J. Oncology 3:473-476). In such an assay, the cells are incubated in media containing the dye, the cells are washed, and the remaining dye, reflecting cellular uptake of the dye, is measured spectrophotometrically. The protein-binding dye sulforhodamine B (SRB) can also be used to measure cytoxicity (Skehan et al., 1990, J. Natl. Cancer Inst. 82:1107-12).

Alternatively, a tetrazolium salt, such as MTT, is used in a quantitative colorimetric assay for mammalian cell survival and proliferation by detecting living, but not dead, cells (see, e.g., Mosmann, 1983, J. Immunol. Methods 65:55-63).

Apoptosis can be quantitated by measuring, for example, DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica, 1999, no. 2, pp. 34-37 (Roche Molecular Biochemicals).

Apoptosis can also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al. eds., 1992, pp. 3.17.1-3.17.16). Cells also can be labeled with a DNA dye (e.g., acridine orange, ethidium bromide, or propidium iodide) and the cells observed for chromatin condensation and margination along the inner nuclear membrane. Other morphological changes that can be measured to determine apoptosis include, e.g., cytoplasmic condensation, increased membrane blebbing, and cellular shrinkage.

The presence of apoptotic cells can be measured in both the attached and “floating” compartments of the cultures. For example, both compartments can be collected by removing the supernatant, trypsinizing the attached cells, combining the preparations following a centrifugation wash step (e.g., 10 minutes at 2000 rpm), and detecting apoptosis (e.g., by measuring DNA fragmentation). (See, e.g., Piazza et al., 1995, Cancer Research 55:3110-16).

The effects of Ligand Drug Conjugates can be tested or validated in animal models. A number of established animal models of cancers are known to the skilled artisan, any of which can be used to assay the efficacy of a Ligand Drug Conjugate. Non-limiting examples of such models are described infra. Moreover, small animal models to examine the in vivo efficacies of Ligand Drug Conjugates can be created by implanting human tumor cell lines into appropriate immunodeficient rodent strains, e.g., athymic nude mice or SCID mice.

### Compositions and Methods of Administration

Various delivery systems are known and can be used to administer the ligand-drug conjugates. Methods of introduction include, but are not limited to, intradermal, intramuscular, intraperitoneal, intravenous, and subcutaneous routes. Administration can be, for example by infusion or bolus injection. In certain preferred embodiments, administration of the Ligand Drug Conjugate is by infusion. Parenteral administration is the preferred route of administration.

The Ligand Drug Conjugates can be administered as pharmaceutical compositions comprising one or more pharmaceutically compatible ingredients. For example, the pharmaceutical composition typically includes one or more pharmaceutical carriers (e.g., sterile liquids, such as water and oils, including those of petroleum, animal, vegetable or synthetic origin, such as peanut oil, soybean oil, mineral oil, sesame oil and the like). Water is a more typical carrier when the pharmaceutical composition is administered intravenously. Saline solutions and aqueous dextrose and glycerol solutions can also be employed as liquid carriers, particularly for injectable solutions. Suitable pharmaceutical excipients are known in the art. The composition, if desired, can also contain minor amounts of wetting or emulsifying agents, or pH buffering agents. Examples of suitable pharmaceutical carriers are described in “Remington's Pharmaceutical Sciences” by E. W. Martin. The formulations correspond to the mode of administration.

In typical embodiments, the pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed, for example, with an infusion bottle containing sterile pharmaceutical grade water or saline. Where the pharmaceutical is administered by injection, an ampoule of sterile water for injection or saline can be, for example, provided so that the ingredients can be mixed prior to administration.

The amount of the compound that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions comprise an effective amount of a compound such that a suitable dosage will be obtained. Typically, this amount is at least about 0.01% of a compound by weight of the composition.

For intravenous administration, the composition can comprise from about 0.01 to about 100 mg of a compound per kg of the animal's body weight. In one aspect, the composition can include from about 1 to about 100 mg of a compound per kg of the animal's body weight. In another aspect, the amount administered will be in the range from about 0.1 to about 25 mg/kg of body weight of a compound.

Generally, the dosage of a compound administered to a patient is typically about 0.01 mg/kg to about 100 mg/kg of the subject's body weight. In some embodiments, the dosage administered to a patient is between about 0.01 mg/kg to about 15 mg/kg of the subject's body weight. In some embodiments, the dosage administered to a patient is between about 0.1 mg/kg and about 15 mg/kg of the subject's body weight. In some embodiments, the dosage administered to a patient is between about 0.1 mg/kg and about 20 mg/kg of the subject's body weight. In some embodiments, the dosage administered is between about 0.1 mg/kg to about 5 mg/kg or about 0.1 mg/kg to about 10 mg/kg of the subject's body weight. In some embodiments, the dosage administered is between about 1 mg/kg to about 15 mg/kg of the subject's body weight. In some embodiments, the dosage administered is between about 1 mg/kg to about 10 mg/kg of the subject's body weight.

The pharmaceutical compositions are generally formulated as sterile, substantially isotonic and in full compliance with all Good Manufacturing Practice (GMP) regulations of the U.S. Food and Drug Administration.

### Therapeutic Methods Using Ligand Drug Conjugates

The Ligand Drug Conjugates are useful for inhibiting the multiplication of a tumor cell or cancer cell, or for treating cancer in an animal. The Ligand Drug Conjugates can be used accordingly in a variety of settings for the treatment of animal cancers.

Particular types of cancers that can be treated with the Ligand Drug Conjugates include, but are not limited to: (1) solid tumors, including but not limited to fibrosarcoma, myxosarcoma, liposarcoma, chondrosarcoma, osteogenic sarcoma, chordoma, angiosarcoma , endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophogeal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma bile duct carcinoma, choriocarcinoma, seminoma, embryonal carcinoma, Wilms' tumor, cervical cancer, uterine cancer, testicular cancer , small cell lung carcinoma, bladder carcinoma, lung cancer, epithelial carcinoma, glioma, glioblastoma, multiforme astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma; (2) blood-borne cancers, including but not limited to acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia , acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, acute and chronic leukemias, e.g., lymphoblastic myelogenous and lymphocytic myelocytic leukemias; and (3) lymphomas such as Hodgkin's disease, non-Hodgkin's Lymphoma, Multiple myeloma, Waldenstrom's macroglobulinemia, Heavy chain disease, and Polycythemia vera.

In some embodiments, the invention provides methods of treating cancer, comprising administering to a subject in need thereof an effective amount of a Ligand Drug Conjugate or a pharmaceutical composition thereof, comprising a DR5 binding agent covalently attached to a cytotoxic agent. In some embodiments, the Ligand Drug Conjugate comprises formula I as provided above. An effective amount of a Ligand Drug Conjugate will be dependent on the subject being treated, the severity of the affliction, and the manner of administration. Determination of an effective amount is well within the capability of those skilled in the art, especially in light of the detailed disclosure provided herein. Generally, an efficacious or effective amount of a Ligand Drug Conjugate is determined by first administering a low dose or small amount, and then incrementally increasing the administered dose or dosages until a desired therapeutic effect is observed in the treated subject, with minimal or no toxic side effects. Applicable methods for determining an appropriate dose and dosing schedule for administration of the present invention are described, for example, in Goodman and Gilman's The Pharmacological Basis of Therapeutics, 11^{th} Ed., Brunton, Lazo and Parker, Eds., McGraw-Hill (2006), and in Remington: The Science and Practice of Pharmacy, 21^{st} Ed., Gennaro, Ed., Lippencott Williams & Wilkins (2003), both of which are hereby incorporated herein by reference.

### EXAMPLES

### Conjugation of DR5 Antibody Drug Conjugates

hTRA-8 antibody drug conjugates were prepared as follows. A hTRA-8 antibody comprising a heavy chain corresponding to the amino acid sequence of SEQ ID NO: 1 and comprising a light chain corresponding to the amino acid sequence of SEQ ID NO: 2 was used as the Ligand unit. This hTRA-8 antibody is referred to as Tigatuzumab. A solution of hTRA-8 antibody at 7.6 mg/mL is pre-equilibrated at 37° C., and then a 15% volume of 500 mM sodium borate, pH 8.0 is added to raise the pH to 7.5-8.0. The solution also contains 1 mM DTPA. The antibody is partially reduced by adding 2.6 equivalents of TCEP per mole of antibody and stirring at 37° C. After 28 minutes, the solution of reduced antibody is placed on ice, then treated immediately with 4.8-4.9 molar equivalents (relative to antibody) of drug linker (e.g., mc-vc-MMAF or mc-vc-MMAE or mc-MMAF) as a 20.5 mM solution in DMSO. Additional DMSO is introduced to bring the mixture to 10% DMSO by volume. The reaction mixture is stirred on ice for ˜90 minutes before treatment with a 5-fold molar excess of N-acetyl cysteine (relative to mc-vc-MMAF). The conjugate is isolated by tangential flow filtration, first being concentrated to ˜10 mg/mL, then diafiltered with ˜10 diavolumes of PBS. The resulting antibody drug conjugates had an average drug loading of about four drug-linker units per antibody. In the attached Figures and the specification, the following abbreviations are used: hTRA-8-vc-MMAF for an antibody drug conjugate of hTRA-8 conjugated with mc-vc-MMAF; hTRA-8-vc-MMAE for an antibody drug conjugate of hTRA-8 conjugated with mc-vc-MMAE; and hTRA-8-mc-MMAF for an antibody drug conjugate of hTRA-8 conjugated with mc-MMAF.

To prepare antibody drug conjugates with an average drug loading of about two drug-linker units per antibody, the protocol (above) was modified by reducing the amount of TCEP by 50%. The amount of drug linker was also reduced by 50%. The corresponding antibody drug conjugate is abbreviated as hTRA-8-vc-MMAF(2).

Cytotoxicity of hTRA-8 ADCs Against Several Human Tumor Cell Lines In Vitro

hTRA-8 and hTRA-8 antibody drug conjugates were diluted with 1 μg/mL of the goat anti-human IgG Fc antibody solution (MP Bioscience) to 2000 ng/mL. These solutions were serially diluted tenfold with the culture medium. An aliquot of 50 μL of each concentration of thses solution was added to a 96-well microplate (Corning). The cell suspension was adjusted to 1.0×10⁵ viable cells/mL of the culture medium and added to the wells at 50 μL/well. The cells were not seeded in the blank wells. After the plates were incubated for 72 h in a CO₂ incubator, ATP detection assay was performed using CellTiter-Glo Luminescent Cell Viability Assay (Promega) according to the manufacturer's instructions. The luminescence was measured by a microplate reader (Mithras LB940, Berthold Technologies). The assay was conducted in triplicate and the cell viability of each well was calculated according to the equation as follows:

Viability(%)=100×(luminescence of a test well−average luminescence of blank wells)/(average luminescence of wells with untreated cells−average luminescence of blank wells).

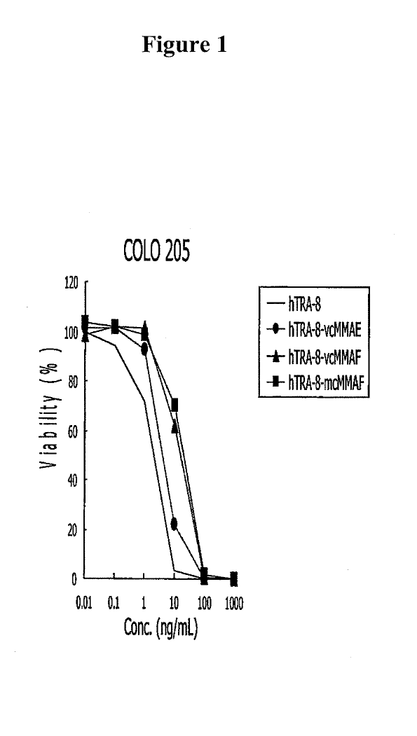

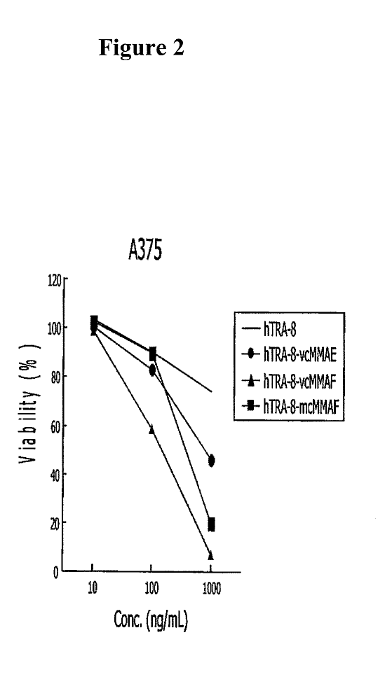

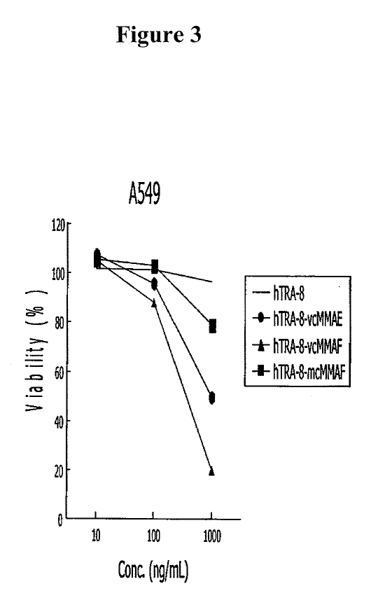

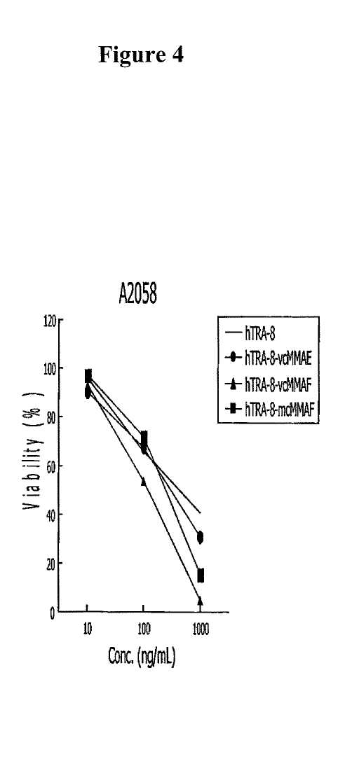

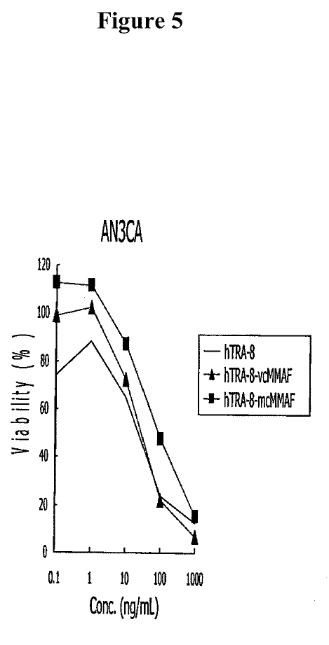

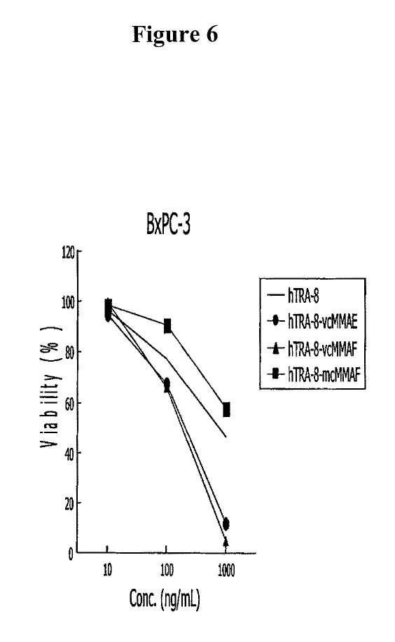

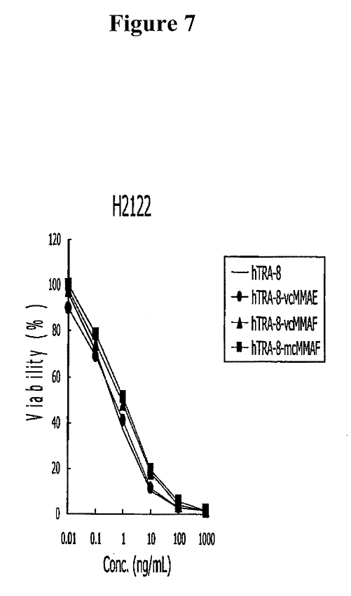

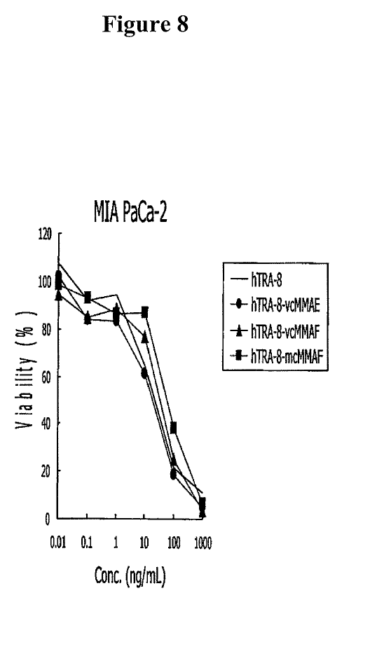

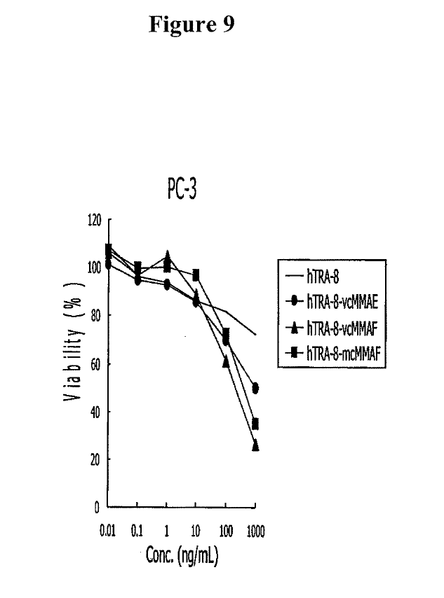

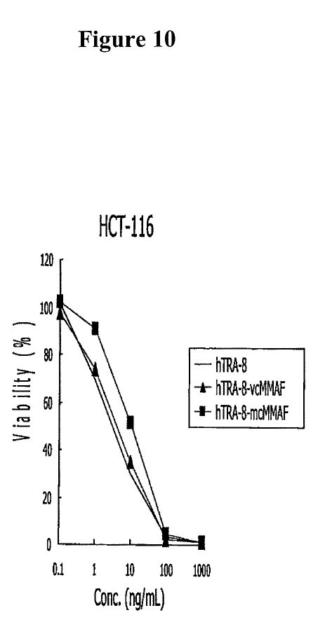

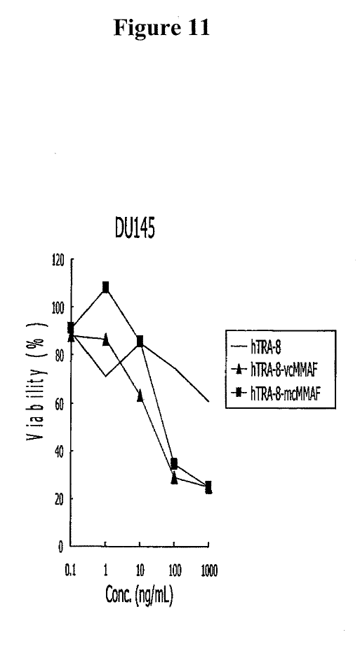

FIGS. 1-11 provide the results for 11 cell lines evaluated with hTRA-8 Ligand Drug Conjugates of the present invention. As the Figures illustrate, these antibody drug conjugates more effectively induced cell death than hTRA-8 (in an unconjugated form) in 6 cell lines among 11 cell lines tested.

DR5 Binding Activity of hTRA-8 ADCs

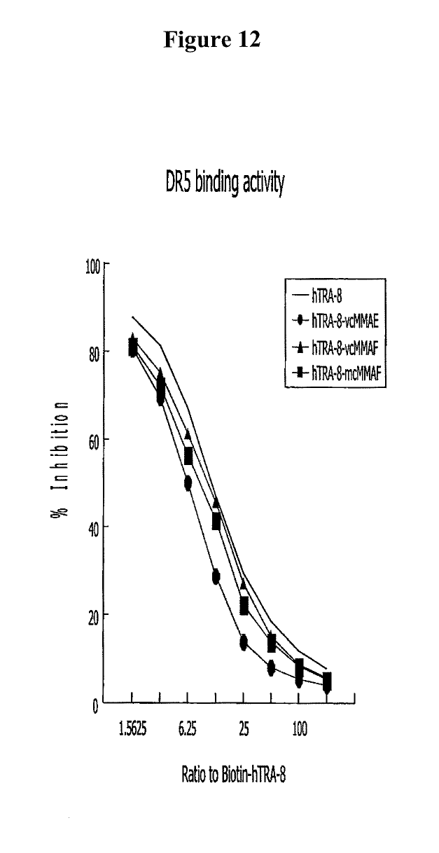

A flat bottom 96-well microplate (Nalge Nunc International) was coated with 0.25 μg/mL of human DR5-Fc in 50 mM NaHCO₃ (pH 9.5) at 4° C. overnight. After washing the wells with 200 μL PBS containing 0.05% Tween 20 (PBS-Tween), the plates were blocked with 100 μL of 1% BSA diluted PBS at room tenperature for 1.5 h. hTRA-8 and hTRA-8 ADCs were serially diluted twofold with PBS from 20 μg/mL to 0.16 μg/mL. After washing the wells with PBS-Tween, 50 μL serial dilutions of hTRA-8 and hTRA-8 ADCs were added to the wells in the presence of 50 μL of biotin-labeled hTRA-8. The plates were incubated at room temperature for 2 h. After the wells were washed with PBS-Tween, 100 μL of streptavidin-horseradish peroxidase conjugate (1/5000 dilution in PBS, Amersham Life Science) was added to the wells, and incubated at room temperature for 1 h. After the wells were washed with PBS-Tween, the color reaction was developed by exposure to 50 μL of HRP substrate solution (Sumilon) at room temperature and the absorbance was measured at 490 nm on a microplate reader (Spectra MAX M5; Molecular Devices). The assay was conducted in triplicate. The results are provided in FIG. 12.

In FIG. 12, the binding activity of hTRA-8 Ligand Drug Conjugates to human DR5 is seen as compared to that of hTRA-8 (in an unconjugated form).

Cytotoxicity of hTRA-8 ADCs Against Human Primary Hepatocytes

For the preparation of primary human hepatocytes, a medium set (Biopredic International) which is consisted of the thawing medium, the seeding medium and the incubation medium was used. The vial of frozen hepatocytes were thawed and washed with the thawing medium. The cells were resuspended in the seeding medium and seeded at 3.5×10⁴ viable cells/well in a collagen-coated 96-well plate (IWAKI). The cells were not seeded in the blank wells. The cells were cultured in a CO₂ incubator. After 4 h of incubation, the culture supernatants were aspirated and 100 μL of the incubation medium was added to each well. After overnight incubation, the culture supernatants were aspirated. hTRA-8 and hTRA-8 antibody drug conjugates were diluted with 0.5 μg/mL of the goat anti-human IgG Fc antibody solution (MP Bioscience) to 1000 ng/mL. These solutions were serially diluted with the culture medium to 100, 10, 1 ng/mL. TRAIL (R&D Systems) was diluted with the incubation medium to 1000, 100, 10, 1 ng/mL. An aliquot of 100 μL of each concentration of these solutions were added to the plates of hepatocytes. After the hepatocytes were incubated for 6 h in a CO₂ incubator, ATP detection assay was performed using CellTiter-Glo Luminescent Cell Viability Assay (Promega) according to the manufacturer's instructions. Luminescence was measured by a microplate reader (Mithras LB940, Berthold Technologies). The assay was conducted in triplicate and the cell viability of each well was calculated according to the equation as follows:

Viability(%)=100×(luminescence of a test well−average luminescence of blank wells)/(average luminescence of wells with untreated cells−average luminescence of blank wells).

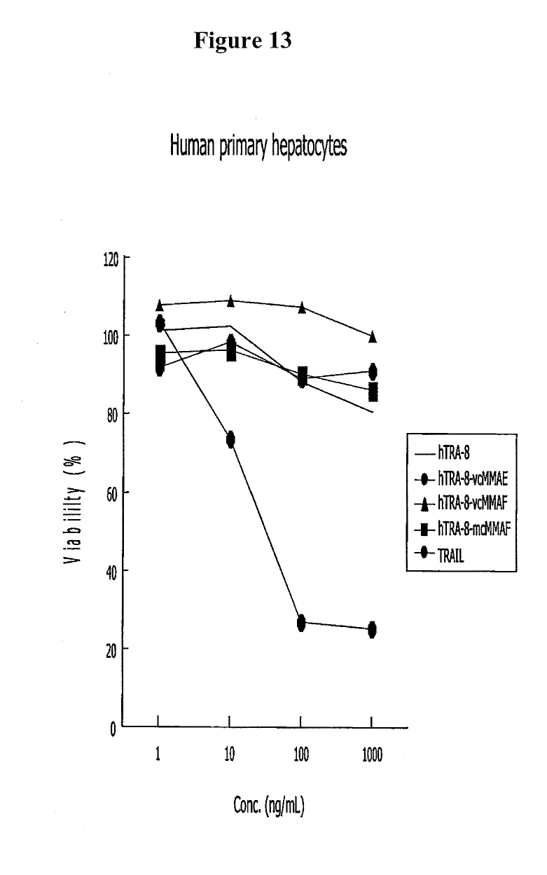

As illustrated in FIG. 13, hTRA-8 Ligand Drug Conjugates did not show cytotoxicity against primary human hepatocytes. (results also with hTRA-8 alone).

### In Vivo Activity for Conjugates—Methods

Specific pathogen-free Balb/cA Jcl nu/nu nude mice (Charles River Laboratories Japan Inc.) aged 6 to 8 weeks were kept for specific pathogen-free condition over 5 days for adaptation before used in studies. Mice were housed in sterilized cages that were placed in a clean laminar airflow rack. Mice were fed with a sterilized solid diet (FR-2, Funabashi Farms Co., Ltd.) and given sterilized tap water prepared with adding 5 to 15 ppm sodium hypochloride solution.

In all the studies, tumor volume (mm³) was calculated by measuring tumor length and tumor width with an electronic digital caliper (CD-15C, Mitutoyo Corp.) two times per week. Calculation of tumor volumes was performed as following equation:

Tumor volume (mm³)=1/2*tumor length (mm)*{tumor width (mm)}²

hTRA-8 and drug-conjugated hTRA-8 were diluted in saline and administered to tumor-bearing nude mice at the volume of 10 mL/kg of mouse body weight.

The detailed procedure of each human tumor xenograft study was described as follows:

COLO205

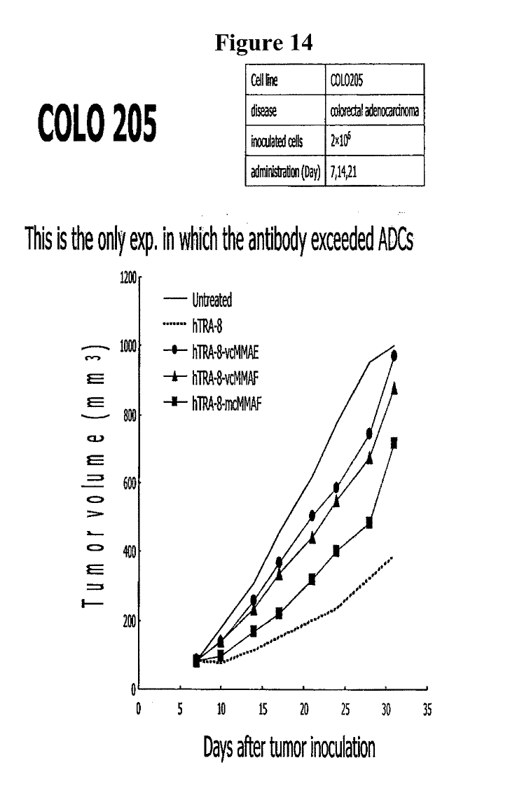

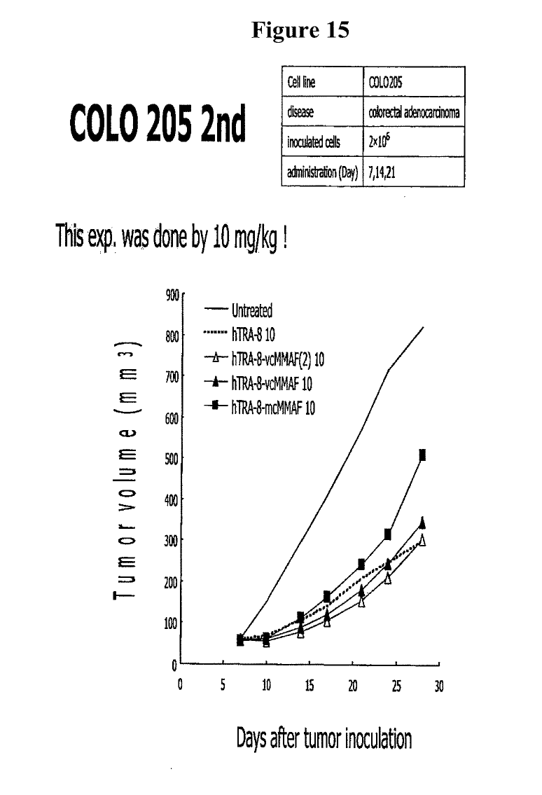

Human colorectal adenocarcinoma cell line COLO205 was purchased from American Type Cell Collection (ATCC). 2 *10⁶ cells were subcutaneously inoculated into right flank of female nude mice on Day 0. On Day 7, all the tumor-bearing nude mice were randomized into experimental groups. In the experiment 1 (FIG. 14), intravenous administration of hTRA-8, hTRA-8-vcMMAE, hTRA-8-vcMMAF and hTRA-8-mcMMAF at the dose of 3 mg/kg was done on Days 7, 14 and 21. In the experiment 2 (FIG. 15), hTRA-8, hTRA-8-vcMMAF(2), hTRA-8-vcMMAF and hTRA-8-mcMMAF were administered intravenously at the dose of 10 mg/kg on Days 7, 14 and 21.

A375

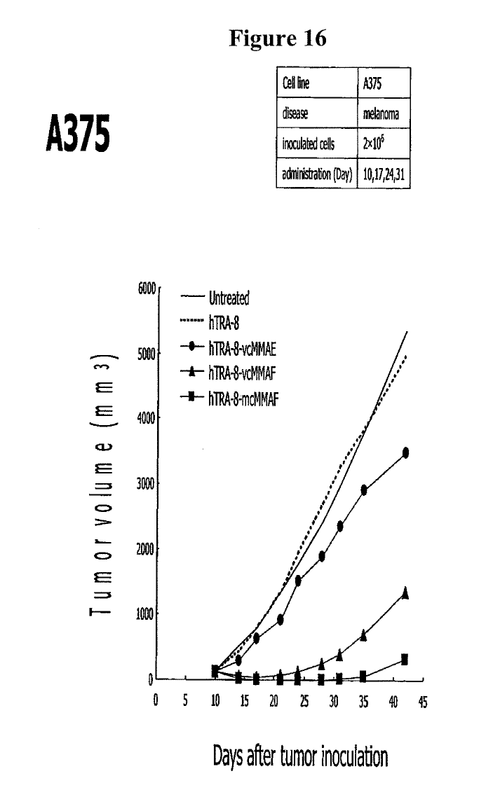

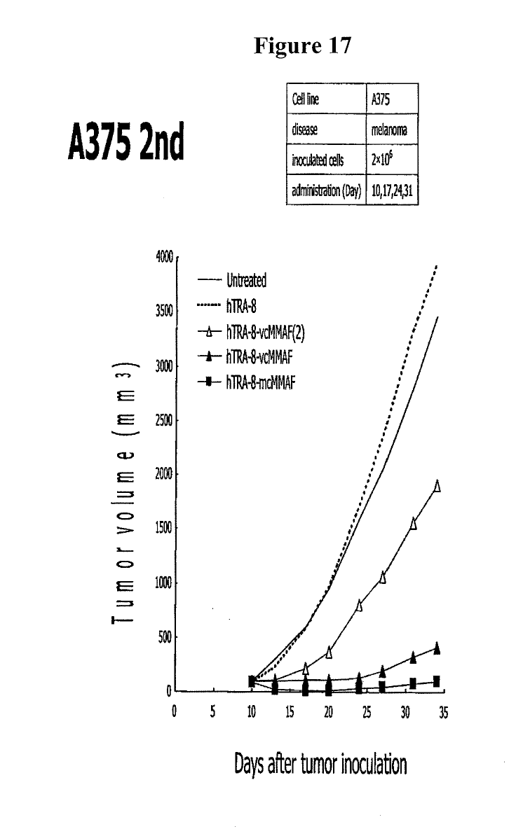

Human melanoma cell line A375 was purchased from American Type Cell Collection (ATCC). 2 *10⁶ cells were subcutaneously inoculated into right flank of female nude mice on Day 0. On Day 10, all the tumor-bearing nude mice were randomized into experimental groups. In experiment 1 (FIG. 16), intravenous administration of hTRA-8, hTRA-8-vcMMAE, hTRA-8-vcMMAF and hTRA-8-mcMMAF at the dose of 3 mg/kg was done on Days 10, 17, 24 and 31. In the experiment 2 (FIG. 17), hTRA-8, hTRA-8-vcMMAF(2), hTRA-8-vcMMAF and hTRA-8-mcMMAF were administered intravenously at the dose of 3 mg/kg on Days 10, 17, 24 and 31

A549

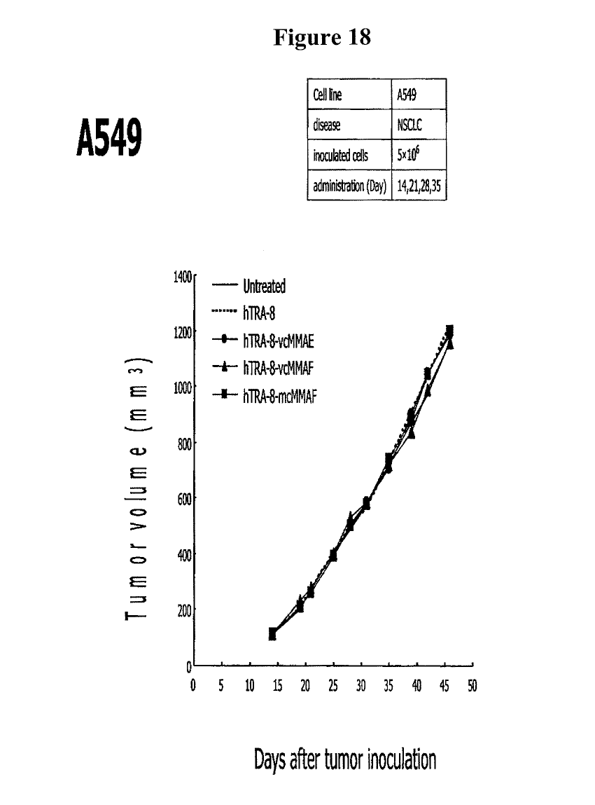

Human lung adenocarcinoma cell line A549 was purchased from American Type Cell Collection (ATCC). 5 *10⁶ cells were subcutaneously inoculated into right flank of female nude mice on Day 0. On Day 14, all the tumor-bearing nude mice were randomized into experimental groups. Intravenous administration of hTRA-8, hTRA-8-vcMMAE, hTRA-8-vcMMAF and hTRA-8-mcMMAF at the dose of 3 mg/kg was done on Day 14, 21, 28 and 35. Results are provided in FIG. 18.

A2058

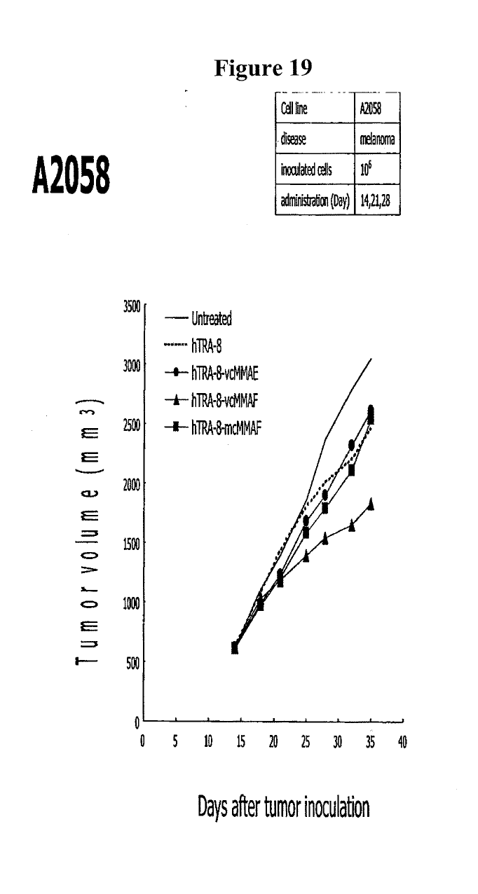

Human melanoma cell line A2058 was purchased from American Type Cell Collection (ATCC). 1 *10⁶ cells were subcutaneously inoculated into right flank of female nude mice on Day 0. On Day 14, all the tumor-bearing nude mice were randomized into experimental groups. Intravenous administration of hTRA-8, hTRA-8-vcMMAE, hTRA-8-vcMMAF and hTRA-8-mcMMAF at the dose of 3 mg/kg was done on Days 14, 21 and 28. Results are provided in FIG. 19.

AN3CA

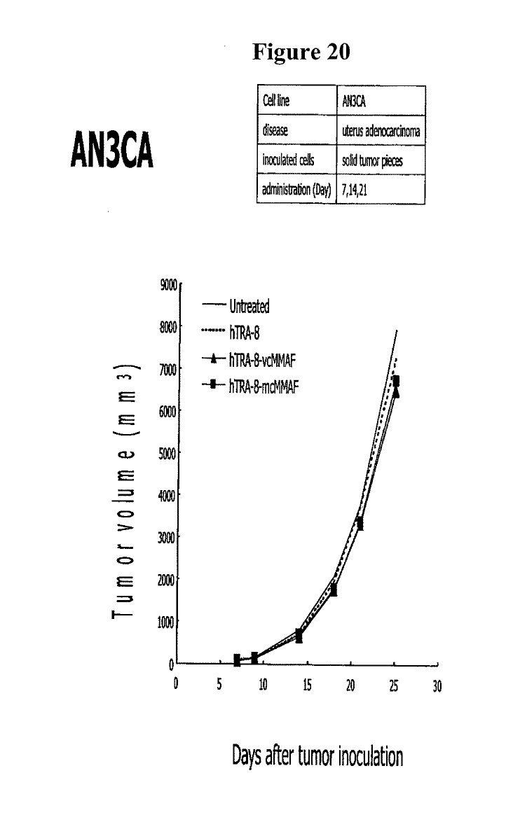

Human uterus adenocarcinoma cell line AN3CA was purchased from American Type Cell Collection (ATCC). Solid tumor pieces (3×3×3 mm³ in size) that had been maintained in nude mice were subcutaneously inoculated into right flank of female nude mice on Day 0. On Day 7, all the tumor-bearing nude mice were randomized into experimental groups. Intravenous administration of hTRA-8, hTRA-8-vcMMAF and hTRA-8-mcMMAF at the dose of 3 mg/kg was done on Days 7, 14 and 21. Results are provided in FIG. 20.

BxPC-3

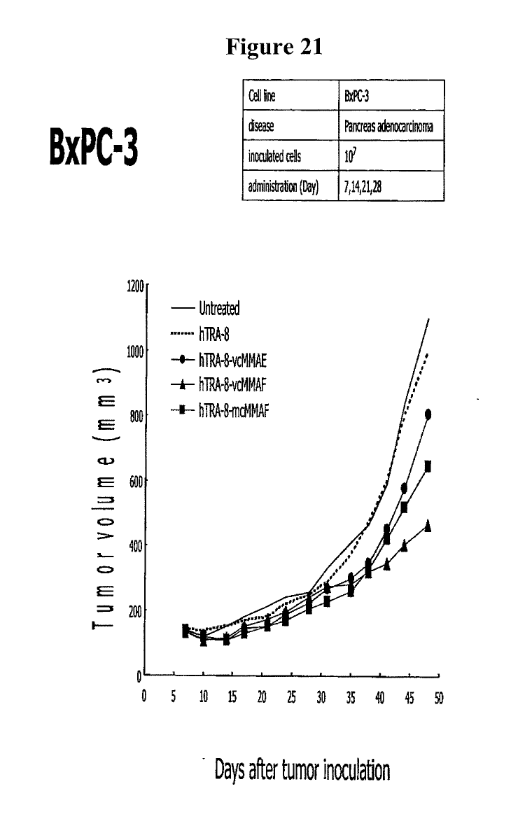

Human pancreas adenocarcinoma cell line BxPC-3 was purchased from American Type Cell Collection (ATCC). 1 *10⁷ cells were subcutaneously inoculated into right flank of female nude mice on Day 0. On Day 7, all the tumor-bearing nude mice were randomized into experimental groups. Intravenous administration of hTRA-8, hTRA-8-vcMMAE, hTRA-8-vcMMAF and hTRA-8-mcMMAF at the dose of 3 mg/kg was done on Days 7, 14, 21 and 28. Results are provided in FIG. 21.

NCI-H2122

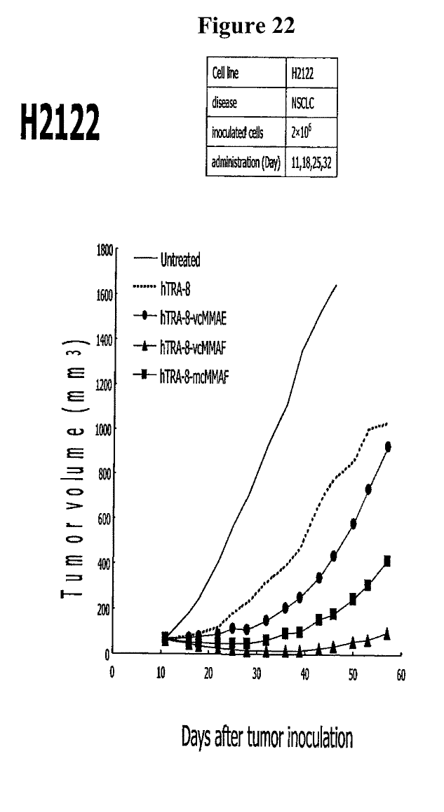

Human lung adenocarcinoma cell line NCI-H2122 was purchased from American Type Cell Collection (ATCC). 2 *10⁶ cells were subcutaneously inoculated into right flank of female nude mice on Day 0. On Day 11, all the tumor-bearing nude mice were randomized into experimental groups. Intravenous administration of hTRA-8, hTRA-8-vcMMAE, hTRA-8-vcMMAF and hTRA-8-mcMMAF at the dose of 3 mg/kg was done on Days 11, 18, 25 and 32. Results are provided in FIG. 22.

MIA PaCa-2

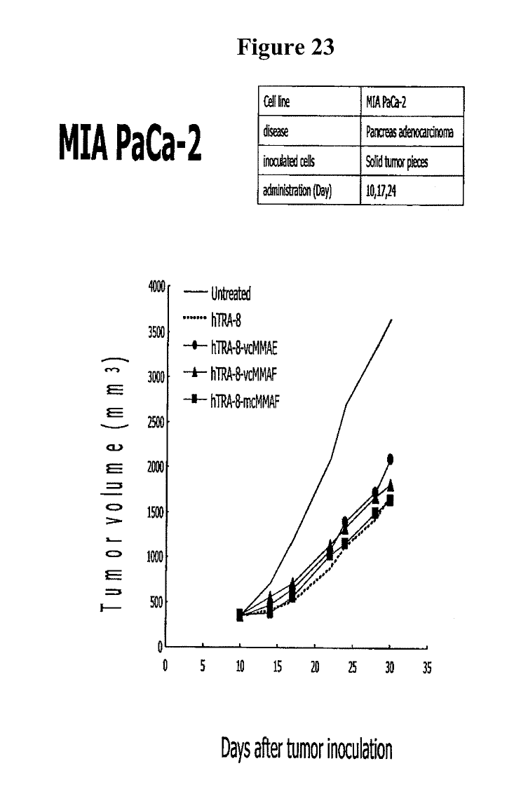

Human pancreas adenocarcinoma cell line MIA PaCa-2 was purchased from American Type Cell Collection (ATCC). Solid tumor pieces (5×5×5 mm³ in size) that had been maintained in nude mice were subcutaneously inoculated into right flank of female nude mice on Day 0. On Day 10, all the tumor-bearing nude mice were randomized into experimental groups. Intravenous administration of hTRA-8, hTRA-8-vcMMAE, hTRA-8-vcMMAF and hTRA-8-mcMMAF at the dose of 3 mg/kg was done on Days 10, 17 and 24. Results are provided in FIG. 23.

PC-3

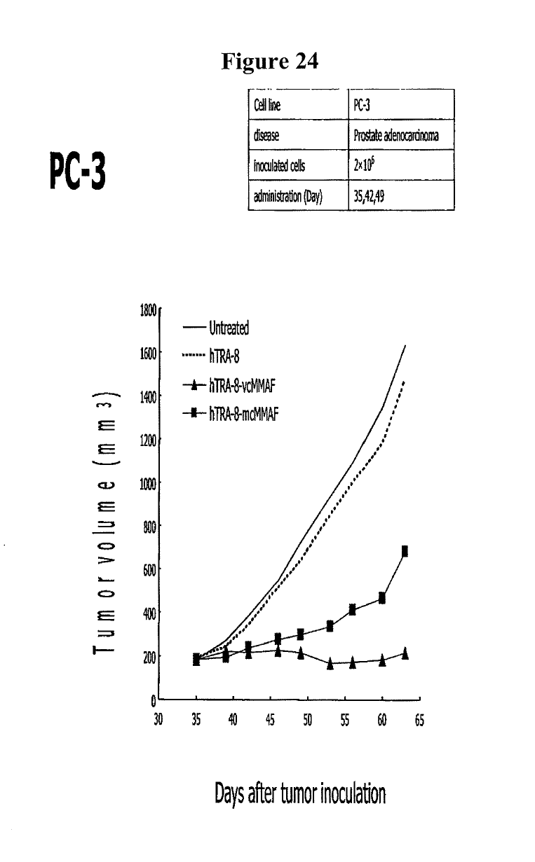

Human prostate adenocarcinoma cell line PC-3 was purchased from American Type Cell Collection (ATCC). 2 *10⁶ cells were subcutaneously inoculated into right flank of male nude mice on Day 0. On Day 35, all the tumor-bearing nude mice were randomized into experimental groups. Intravenous administration of hTRA-8, hTRA-8-vcMMAF and hTRA-8-mcMMAF at the dose of 3 mg/kg was done on Days 35, 42 and 49. Results are provided in FIG. 24.

HCT-116

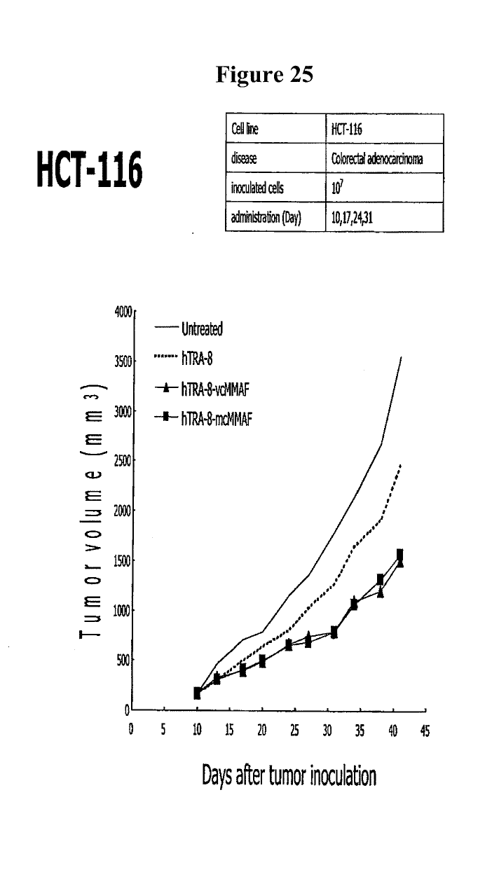

Human colorectal adenocarcinoma cell line HCT-116 was purchased from American Type Cell Collection (ATCC). 1 *10⁷ cells were subcutaneously inoculated into right flank of female nude mice on Day 0. On Day 10, all the tumor-bearing nude mice were randomized into experimental groups. Intravenous administration of hTRA-8, hTRA-8-vcMMAF and hTRA-8-mcMMAF at the dose of 3 mg/kg was done on Days 10, 17, 24 and 31. Results are provided in FIG. 25.

DU145

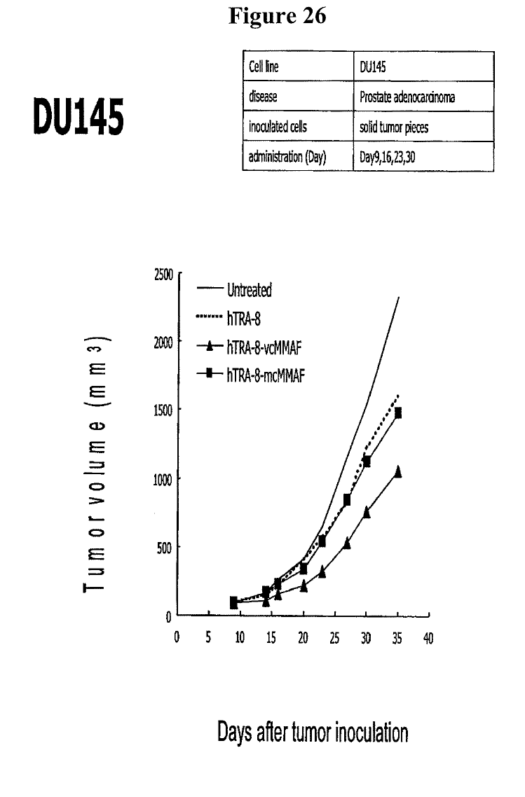

Human prostate adenocarcinoma cell line DU145 was purchased from American Type Cell Collection (ATCC). Solid tumor pieces (5×5×5 mm³ in size) that had been maintained by subcutaneously implanted into nude mice were subcutaneously inoculated into right flank of male nude mice on Day 0. On Day 9, all the tumor-bearing nude mice were randomized into experimental groups. Intravenous administration of hTRA-8, hTRA-8-vcMMAF and hTRA-8-mcMMAF at the dose of 3 mg/kg was done on Days 9, 16, 23 and 30. Results are provided in FIG. 26.

### In Vivo Activity for Conjugates—Results

Among 11 tumor cell lines tested, A375, A549, A2058, AN3CA, BXPC-3, PC-3, HCT-116 and DU145 were demonstrated to be resistant to hTRA-8. Among these 8 tumor cell lines, both hTRA-8-vcMMAF and hTRA-8-mcMMAF showed anti-tumor efficacy against A375, PC-3 and HCT-116. In addition, hTRA-8-vcMMAF also showed anti-tumor efficacy against A2058, BXPC-3 and DU145. hTRA-8 showed moderate anti-tumor efficacy against NCI-H2122, while hTRA-8-vcMMAF and hTRA-8-mcMMAF demonstrated more potent anti-tumor efficacy than hTRA-8. On the other hand, all the drug-conjugated hTRA-8 showed less potent anti-tumor efficacy to COLO205 than hTRA-8 at the dose of 3 mg/kg. When administration doses were increased to 10 mg/kg, hTRA-8-vcMMAF and hTRA-8-mcMMAF showed comparable efficacy to hTRA-8. A549 and AN3CA were resistant to hTRA-8 and drug-conjugated hTRA-8. From these results, hTRA-8 Ligand Drug Conjugates were shown to have more potent anti-tumor efficacy than hTRA-8 and demonstrate efficacy to hTRA-8 resistant tumors.

### In Vivo Competition Study—Methods

Specific pathogen-free female CAnN.Cg-FoxnInu/CrlCrlj mice (nude mice), aged 4 to 6 weeks, were purchased from Charles River Laboratories Japan Inc., and were used when they reached 5 to 8 weeks of age. Five to six mice were housed together in sterilized cages and maintained under specific pathogen-free conditions. In the experimental room, the environmental conditions were set at a temperature of 23° C. and 55% humidity with artificial illumination of 12 h (8:00-20:00). The mice were fed an FR-2 diet (Funabashi Farm Co., Ltd.) and provided with water with chlorine (5-15 ppm) ad libitum.

In all the studies, tumor-bearing mice were selected and divided into experimental groups based on the tumor volume. After the establishment of the tumors on the nude mice, tumor length and width (mm) in all the tumor-bearing mice were measured with a digital caliper (CD15-C, Mituyo Corp.) to two decimal places. The data were automatically recorded in the Sankyo management system for animal experimental data (SMAD, JMAC Corp.). The tumor volume of each mouse as automatically calculated in SMAD according to the following equation:

Tumor volume (mm³)=1/2*tumor length (mm)*{tumor width (mm)}²

Recombinant human DR5-Fc (rhDRS-Fc), human IgG (hIgG), drug-conjugated hIgG and drug-conjugated hTRA-8 were diluted in saline and administered to tumor-bearing nude mice at the volume of 10 mL/kg of mouse body weight. The detailed procedure of each human tumor xenograft study is described as follows.

A375

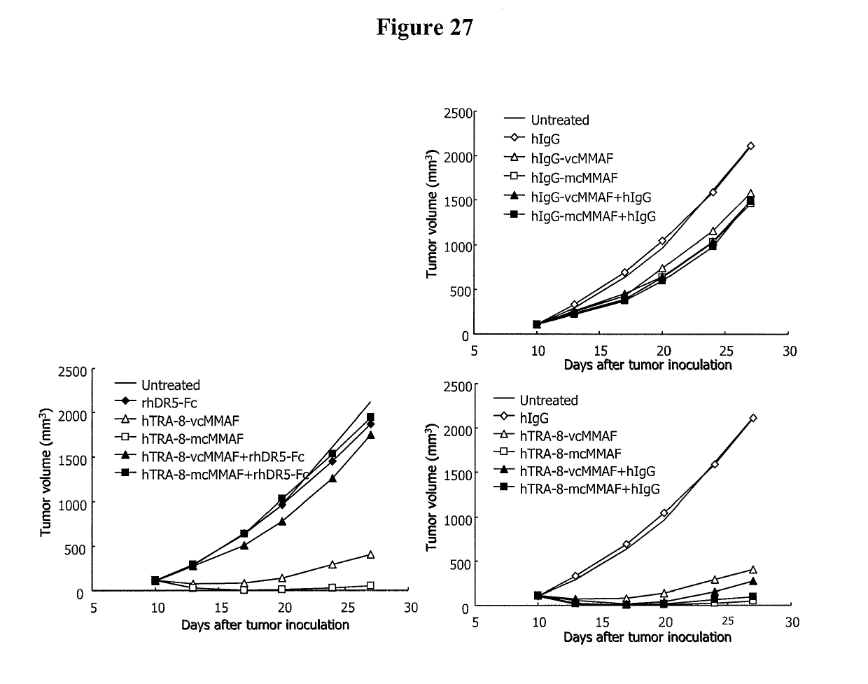

Human melanoma cell line A375 was purchased from American Type Culture Collection (ATCC). On Day 0, 2×10⁶ cells were subcutaneously inoculated into the right flank of nude mice. All the tumor-bearing mice were divided into the experimental groups on Day 10. Just before the administration of ADCs, rhDR5-Fc and hIgG were intravenously administered into the mice at the dose of 3 mg/kg. Then, hIgG-vcMMAF (hIgG conjugated with mc-vc-MMAF) and hIgG-mcMMAF (hIgG conjugated with mc-MMAF) were administered into the mice at the dose of 10 mg/kg, and hTRA-8-vcMMAF and hTRA-8-mcMMAF were administered into the mice at the dose of 3 mg/kg. On Days 11-14 and 17-21, 1 mg/kg of rhDR5-Fc and hIgG were intravenously administered into the mice. Results are provided in FIG. 27.

HCT 116

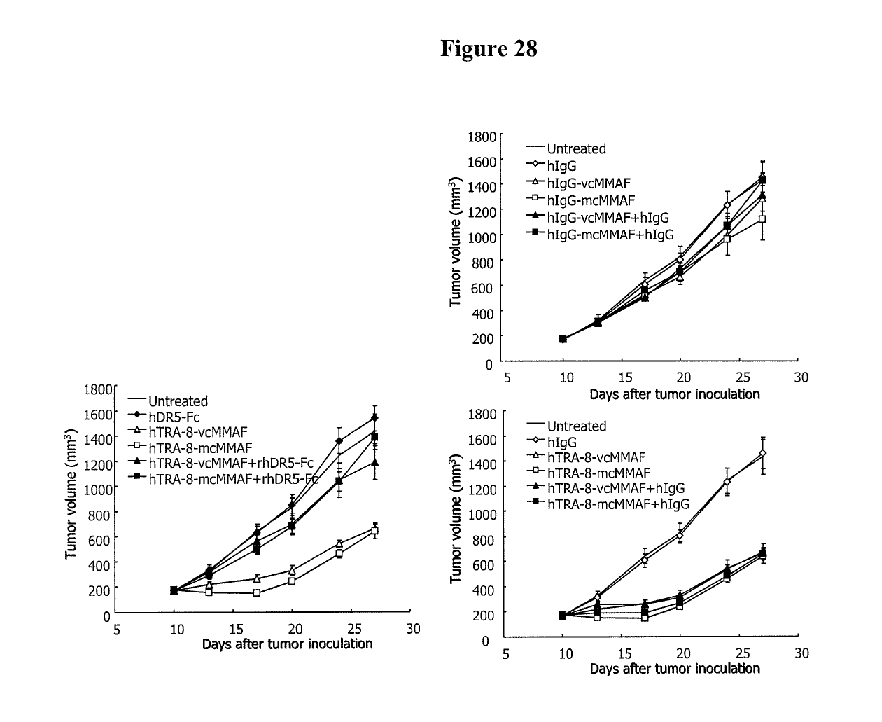

Human colorectal carcinoma cell line HCT 116 was purchased from American Type Culture Collection (ATCC). On Day 0, 1×10⁷ cells were subcutaneously inoculated into the right flank of nude mice. All the tumor-bearing mice were divided into the experimental groups on Day 10. Just before the administration of ADCs, 6 mg/kg of rhDR5-Fc and 10 mg/kg of hIgG were intravenously administered into the mice. Then, hIgG-vcMMAF, hIgG-mcMMAF, hTRA-8-vcMMAF, and hTRA-8-mcMMAF were administered into the mice at the dose of 10 mg/kg. On Days 11-14 and 17-21, 1 mg/kg of rhDR5-Fc and 2 mg/kg of hIgG were intravenously administered into the mice. Results are provided in FIG. 28.

### In Vivo Competition Study—Results

In both xenograft models, rhDR5-Fc completely inhibited the anti-tumor efficacies of hTRA-8-vcMMAF and hTRA-8-mcMMAF. However, hIgG did not inhibit the anti-tumor efficacies of hIgG-vcMMAF, hIgG-mcMMAF, hTRA-8-vcMMAF, and hTRA-8-mcMMAF. These results indicate that the anti-tumor efficacies of hTRA-8-vcMMAF and hTRA-8-mcMMAF are specific to hDR5.

### In Vivo Activity Against Breast and Ovarian Cancers—Methods

Specific pathogen-free female CAnN.Cg-Foxn1nu/CrlCrlj mice (nude mice), aged 4 to 6 weeks, were purchased from Charles River Laboratories Japan Inc., and were used when they reached 5 to 8 weeks of age. Five to six mice were housed together in sterilized cages and maintained under specific pathogen-free conditions. In the experimental room, the environmental conditions were set at a temperature of 23° C. and 55% humidity with artificial illumination of 12 h (8:00-20:00). The mice were fed an FR-2 diet (Funabashi Farm Co., Ltd.) and provided with water with chlorine (5-15 ppm) ad libitum.

In all the studies, tumor-bearing mice were selected and divided into experimental groups based on the tumor volume. After the establishment of the tumors on the nude mice, tumor length and width (mm) in all the tumor-bearing mice were measured with a digital caliper (CD15-C, Mituyo Corp.) to two decimal places. The data were automatically recorded in the Sankyo management system for animal experimental data (SMAD, JMAC Corp.). The tumor volume of each mouse as automatically calculated in SMAD according to the following equation:

Tumor volume (mm³)=1/2*tumor length (mm)*{tumor width (mm)}²

hTRA-8-mcMMAF was diluted in saline and intravenously administered to tumor-bearing nude mice at the volume of 10 mL/kg of mouse body weight. The detailed procedure of each human tumor xenograft study is described as follows.

JIMT-1

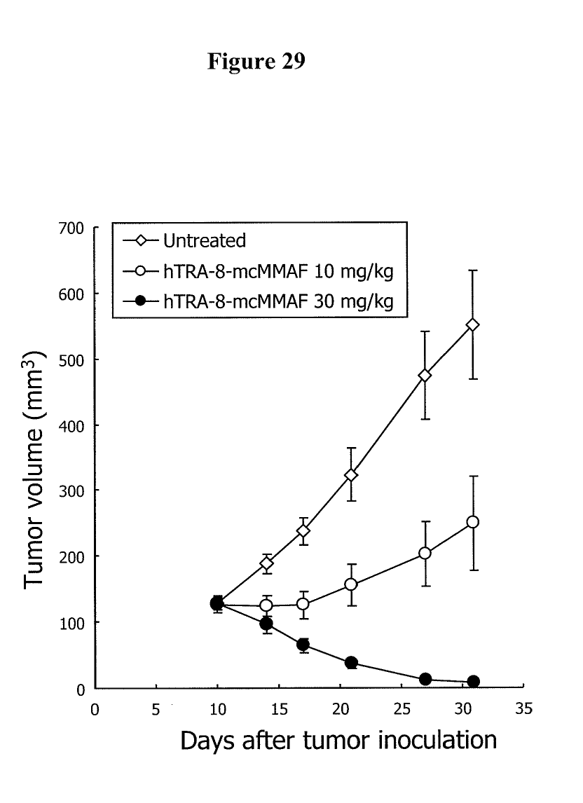

Human breast carcinoma cell line JIMT-1 was purchased from Deutsche Sammlung von Mikroorganismen and Zellkulturen GmbH (DSMZ, German Collection of Microorganisms and Cell Cultures). On Day 0, 6×10⁶ cells were subcutaneously inoculated into the right flank of nude mice. All the tumor-bearing mice were divided into the experimental groups on Day 10. On Days 10, 17, and 24, 10 and 30 mg/kg of hTRA-8-mcMMAF was intravenously administered into the mice. Results are provided in FIG. 29.

MDA-MB-231

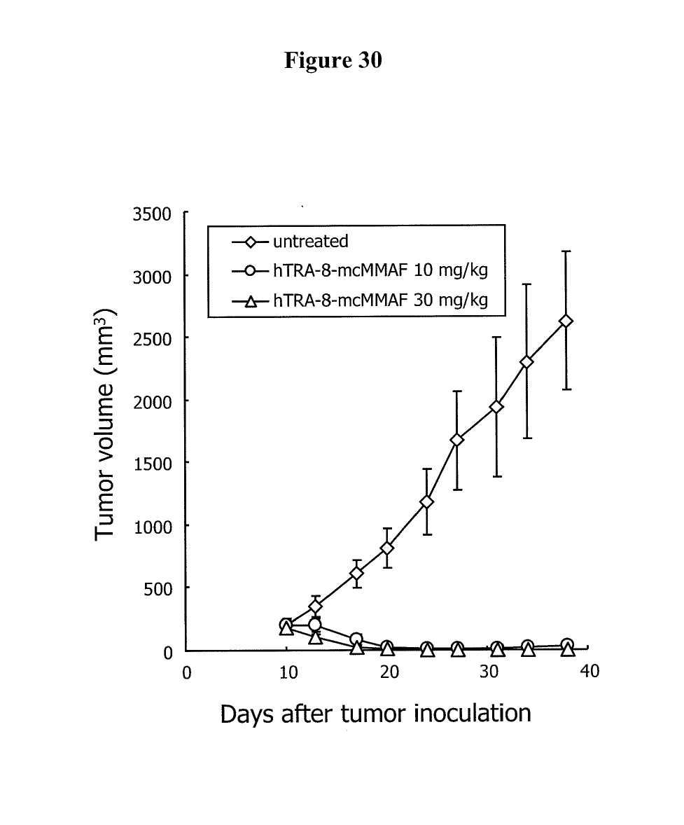

Human breast adenocarcinoma cell line MDA-MB-231 was purchased from American Type Culture Collection (ATCC). On Day 0, solid tumor pieces (approximately 5 mm on a side) that had been maintained in nude mice were subcutaneously inoculated into the right flank of nude mice. On Days 10, 17, and 24, 10 and 30 mg/kg of hTRA-8-mcMMAF was intravenously administered into the mice. Results are provided in FIG. 30.

A2780

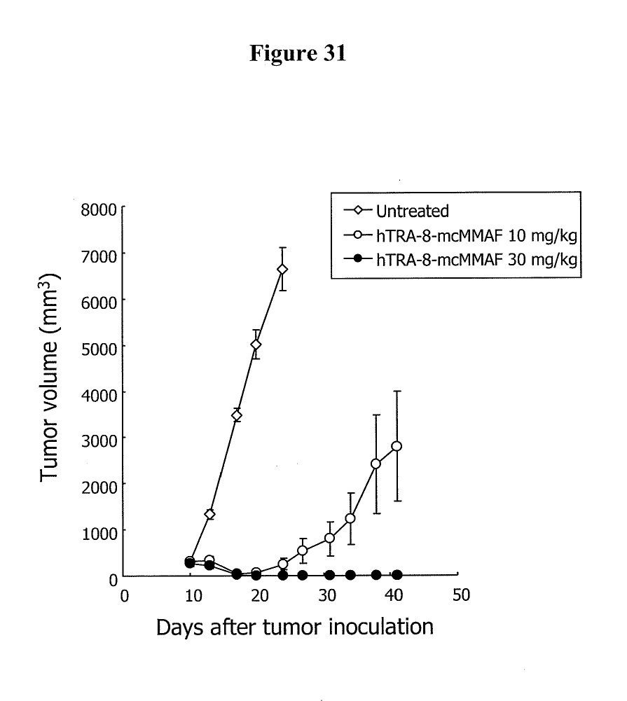

Human ovarian adenocarcinoma cell line A2780 was purchased from European Collection of Cell Cultures (ECACC). On Day 0, 5×10⁶ cells were subcutaneously inoculated into the right flank of nude mice. All the tumor-bearing mice were divided into the experimental groups on Day 10. On Days 10, 17, and 24, 10 and 30 mg/kg of hTRA-8-mcMMAF was intravenously administered into the mice. Results are provided in FIG. 31.

SK-OV-3

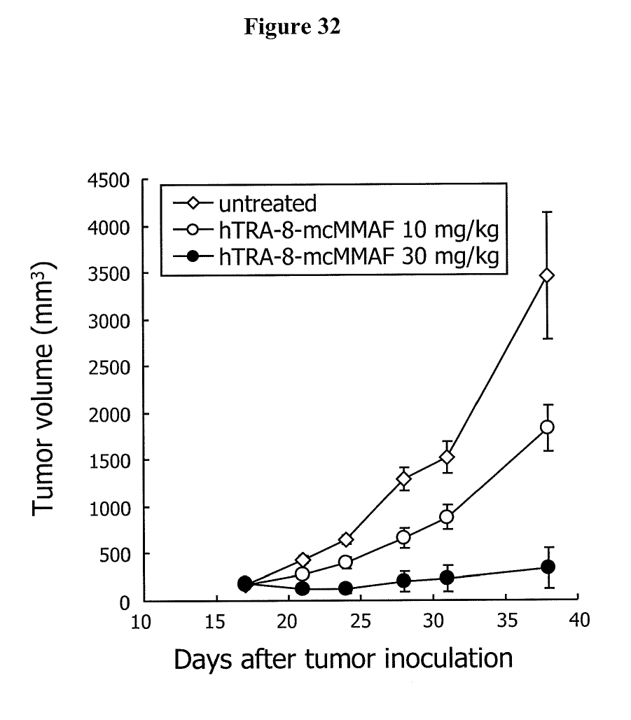

Human ovarian adenocarcinoma cell line SK-OV-3 was purchased from American Type Culture Collection (ATCC). On Day 0, solid tumor pieces (approximately 5 mm on a side) that had been maintained in nude mice were subcutaneously inoculated into the right flank of nude mice. On Days 17, 24, and 31, 10 and 30 mg/kg of hTRA-8-mcMMAF was intravenously administered into the mice. Results are provided in FIG. 32.

### In Vivo Activity Against Breast and Ovarian Cancers—Results

hTRA-8-mcMMAF showed anti-tumor efficacy in JIMT-1, MDA-MB-231, A2780, and SK-OV-3 xenograft mice. From these results, hTRA-8-mcMMAF was indicated to have potent anti-tumor activity against breast and ovarian cancers.

### In Vivo Activity Against Hematological Cancers—Methods

Specific pathogen-free female NOD.CB17-Prkdc^{scid}/J mice (NOD-scid mice), aged 4 to 6 weeks, were purchased from Charles River Laboratories Japan Inc., and were used when they reached 5 to 8 weeks of age. Five to six mice were housed together in sterilized cages and maintained under specific pathogen-free conditions. In the experimental room, the environmental conditions were set at a temperature of 23° C. and 55% humidity with artificial illumination of 12 h (8:00-20:00). The mice were fed an FR-2 diet (Funabashi Farm Co., Ltd.) and provided with water with chlorine (5-15 ppm) ad libitum.

In all the studies, all the mice were randomly divided into experimental groups on Day 7. Then, hTRA-8-mcMMAF was diluted in saline and intravenously administered to the mice at the volume of 10 mL/kg of mouse body weight. The detailed procedure of each human tumor xenograft study is described as follows.

U-937

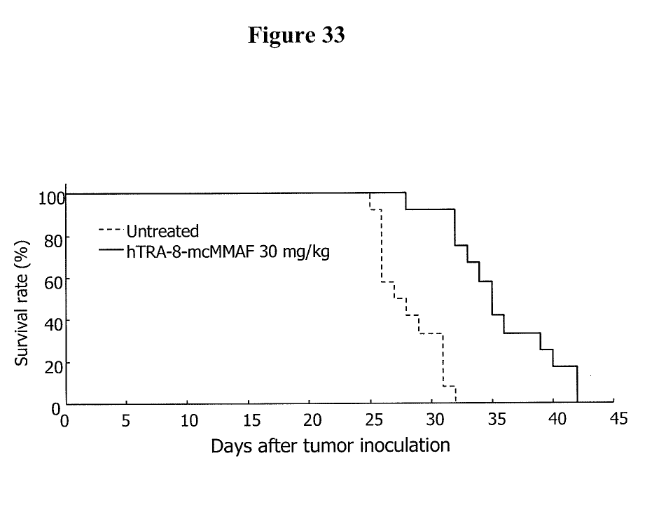

Human histiocytic lymphoma cell line U-937 was purchased from American Type Culture Collection (ATCC). On Day 0, 1×10⁷ cells were intravenously inoculated into the mice. On Days 7, 14, and 21, 30 mg/kg of hTRA-8-mcMMAF was intravenously administered into the mice. Results are provided in FIG. 33.

MOLT-4

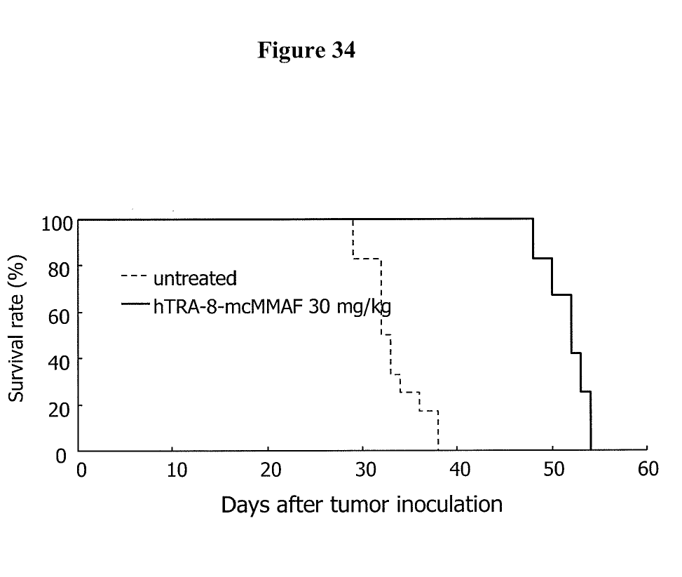

Human acute lymphoblastic leukemia cell line MOLT-4 was purchased from American Type Culture Collection (ATCC). On Day 0, 5×10⁶ cells were intravenously inoculated into the mice which were previously treated with intravenous administration of 150 mg/kg of cyclophosphamide on Days −2 and −1. On Days 7, 14, 21, and 28, 30 mg/kg of hTRA-8-mcMMAF was intravenously administered into the mice. Results are provided in FIG. 34.

MOLM-14

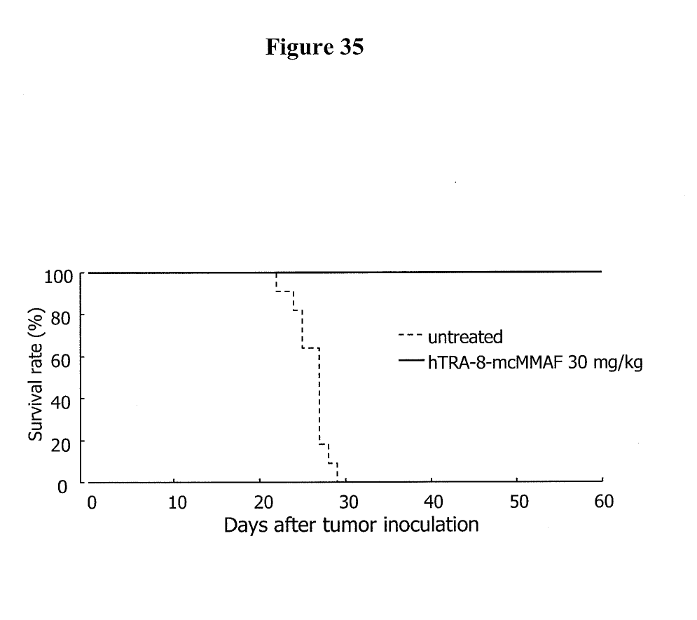

Human acute monocytic leukemia cell line MOLM-14 was obtained from Hayashibara Biochemical Labs, Inc. On Day 0, 5×10⁶ cells were intravenously inoculated into the mice which were previously treated with intravenous administration of 150 mg/kg of cyclophosphamide on Days −2 and −0.1. On Days 7, 14, and 21, 30 mg/kg of hTRA-8-mcMMAF was intravenously administered into the mice. Results are provided in FIG. 35.

MV-4-11

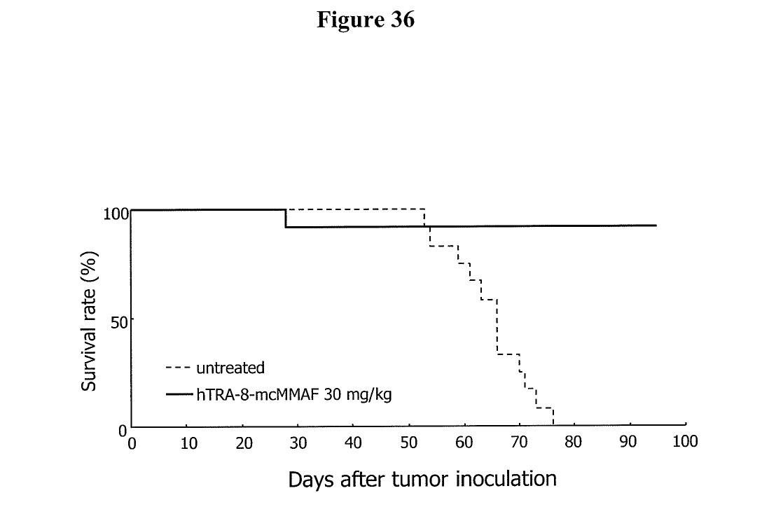

Human myelomonocytic leukemia cell line MV-4-11 was purchased from American Type Culture Collection (ATCC). On Day 0, 5×10⁶ cells were intravenously inoculated into the mice. On Days 7, 14, 21, 28, 35, 42, and 49, 30 mg/kg of hTRA-8-mcMMAF was intravenously administered into the mice. Results are provided in FIG. 36.

### In Vivo Activity Against Hematological Cancers—Results

hTRA-8-mcMMAF prolonged the life-span of the mice which were intravenously inoculated with the hematological cancers MOLM-14, U-937, MV-4-11, and MOLT-4. From these results, hTRA-8-mcMMAF was indicated to have potent anti-tumor activity against hematological cancers.

1. A ligand drug conjugate comprising a DR5 binding agent covalently attached to a cytotoxic agent.

2. A ligand drug conjugate having Formula (I):
L-(LU-D)ₚ (I)
or a pharmaceutically acceptable salt thereof, having specificity for target cells expressing DR5, wherein
L is a ligand unit which is a DR⁵ binding agent; and
(LU-D) is a Linker unit-Drug unit moiety, wherein
LU is a Linker unit, and
D is a Drug unit having cytostatic or cytotoxic activity against said target cells; and
the subscript p is an integer of from 1 to 20.

3. The ligand-drug conjugate of claim 2, wherein the Drug unit has formula D_{E} or D_{F}: 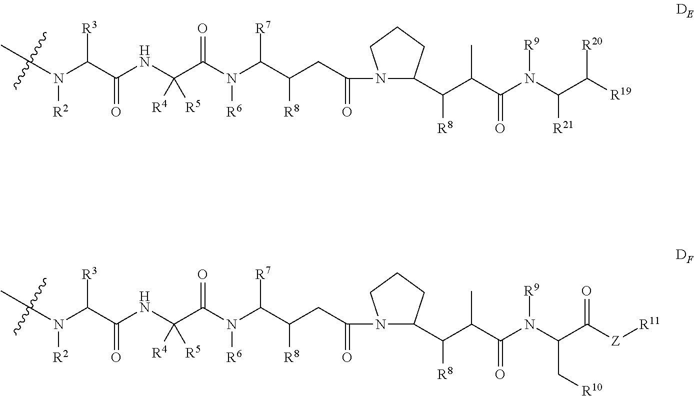
or a pharmaceutically acceptable salt form thereof;
wherein, independently at each location:
the wavy line indicates the point of attachment to the remainder of the ligand drug conjugate;
R² is —C₁-C₂₀ alkyl, —C₂-C₂₀ alkenyl, or —C₂-C₂₀ alkynyl;
R³ is —H, —C₁-C₂₀ alkyl, —C₂-C₂₀ alkenyl, —C₂-C₂₀ alkynyl, carbocycle, —C₁-C₂₀ alkylene (carbocycle), —C₂-C₂₀ alkenylene(carbocycle), —C₂-C₂₀ alkynylene(carbocycle), -aryl, —C₁-C₂₀ alkylene(aryl), —C₂-C₂₀ alkenylene(aryl), —C₂-C₂₀ alkynylene(aryl), -heterocycle, —C₁-C₂₀ alkylene(heterocycle), —C₂-C₂₀ alkenylene(heterocycle), or —C₂-C₂₀ alkynylene(heterocycle);
R⁴ is —H, —C₁-C₂₀ alkyl, —C₂-C₂₀ alkenyl, —C₂-C₂₀ alkynyl, carbocycle, —C₁-C₂₀ alkylene (carbocycle), —C₂-C₂₀ alkenylene(carbocycle), —C₂-C₂₀ alkynylene(carbocycle), -aryl, —C₁-C₂₀ alkylene(aryl), —C₂-C₂₀ alkenylene(aryl), —C₂-C₂₀ alkynylene(aryl), -heterocycle, —C₁-C₂₀ alkylene(heterocycle), —C₂-C₂₀ alkenylene(heterocycle), or —C₂-C₂₀ alkynylene(heterocycle);
R⁵ is —H or —C₁-C₈ alkyl;
or R⁴ and R⁵ jointly form a carbocyclic ring and have the formula —(CR^{a}R^{b})ₛ— wherein R^{a} and R^{b} are independently —H, —C₁-C₂₀ alkyl, —C₂-C₂₀ alkenyl, —C₂-C₂₀ alkynyl, or -carbocycle and s is 2, 3, 4, 5 or 6,
R⁶ is —H, —C₁-C₂₀ alkyl, —C₂-C₂₀ alkenyl, or —C₂-C₂₀ alkynyl;
R⁷ is —H, —C₁-C₂₀ alkyl, —C₂-C₂₀ alkenyl, —C₂-C₂₀ alkynyl, -carbocycle, —C₁-C₂₀ alkylene (carbocycle), —C₂-C₂₀ alkenylene(carbocycle), —C₂-C₂₀ alkynylene(carbocycle), -aryl, —C₁-C₂₀ alkylene(aryl), —C₂-C₂₀ alkenylene(aryl), —C₂-C₂₀ alkynylene(aryl), heterocycle, —C₁-C₂₀ alkylene(heterocycle), —C₂-C₂₀ alkenylene(heterocycle), or —C₂-C₂₀ alkynylene(heterocycle);
each R⁸ is independently —H, —OH, —C₁-C₂₀ alkyl, —C₂-C₂₀ alkenyl, —C₂-C₂₀ alkynyl, —O—(C₁-C₂₀ alkyl), —O—(C₂-C₂₀ alkenyl), —O—(C₂-C₂₀ alkynyl), or -carbocycle;
R⁹ is —H, —C₁-C₂₀ alkyl, —C₂-C₂₀ alkenyl, or —C₂-C₂₀ alkynyl;
R¹⁹ is -aryl, -heterocycle, or -carbocycle;
R²⁰ is —H, —C₁-C₂₀ alkyl, —C₂-C₂₀ alkenyl, —C₂-C₂₀ alkynyl, -carbocycle, —O—(C₁-C₂₀—O—(C₂-C₂₀ alkenyl), —O—(C₂-C₂₀ alkynyl), or OR¹⁸ wherein R¹⁸ is —H, a hydroxyl protecting group, or a direct bond where OR¹⁸ represents ═O;
R²¹ is —H, —C₁-C₂₀ alkyl, —C₂-C₂₀ alkenyl, or —C₂-C₂₀ alkynyl, -aryl, heterocycle, or -carbocycle;
R¹⁰ is -aryl or -heterocycle;
Z is —O—, —S—, —NH—, or —NR¹²—, wherein R¹² is —C₁-C₂₀ alkyl, —C₂-C₂₀ alkenyl, or —C₂-C₂₀ alkynyl;
R¹¹ is —H, —C₁-C₂₀ alkyl, —C₂-C₂₀ alkenyl, —C₂-C₂₀ alkynyl, -aryl, -heterocycle, —(R¹³O)ₘ—R¹⁴, or —(R¹³O)ₘCH(R¹⁵)₂;
m is an integer ranging from 0-1000;
R¹³ is —C₂-C₂₀ alkylene, —C₂-C₂₀ alkenylene, or —C₂-C₂₀ alkynylene;
R¹⁴ is —H, —C₁-C₂₀ alkyl, —C₂-C₂₀ alkenyl, or —C₂-C₂₀ alkynyl;
each occurrence of R¹⁵ is independently —H, —COOH, —(CH₂)ₙ—N(R¹⁶)₂, —(CH₂)ₙ—SO₃H, —(CH₂)ₙ—SO₃—C₁-C₂₀ alkyl, —(CH₂)ₙ—SO₃—C₂-C₂₀ alkenyl, or —(CH₂)ₙ—SO₃—C₂-C₂₀ alkynyl;
each occurrence of R¹⁶ is independently —H, —C₁-C₂₀ alkyl, —C₂-C₂₀ alkenyl, —C₂-C₂₀ alkynyl or —(CH₂)ₙ—COOH; and
n is an integer ranging from 0 to 6; wherein said alkyl, alkenyl, alkynyl, alkylene, alkenylene, alkynyklene, aryl, carbocyle, and heterocycle radicals, whether alone or as part of another group, are optionally substituted.

4. The ligand drug conjugate of claim 3, wherein the Drug unit has formula D_{E} or a pharmaceutically acceptable salt form thereof.

5. The ligand drug conjugate of claim 3, wherein the Drug unit has the formula: 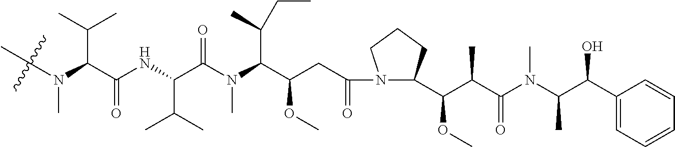
or a pharmaceutically acceptable salt form thereof; the DR5 binding agent is an anti-DR5 antibody attached to the Linker unit through a sulfur atom of the antibody; the Linker unit comprises a -Val-Cit-moiety; and the subscript p is an integer of from 1 to 8.

6. The ligand drug conjugate of claim 3, wherein the Drug unit has the formula: 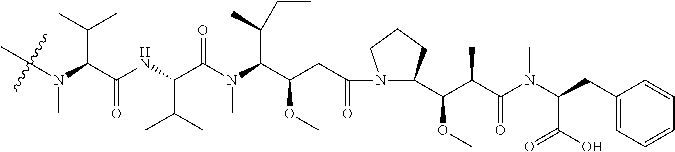
or a pharmaceutically acceptable salt form thereof; the DR5 binding agent is an anti-DR5 antibody attached to the Linker unit through a sulfur atom of the antibody; the Linker unit comprises a -Val-Cit-moiety; and the subscript p is an integer of from 1 to 8.

7. The ligand drug conjugate of claim 3, wherein the Drug unit has the formula: 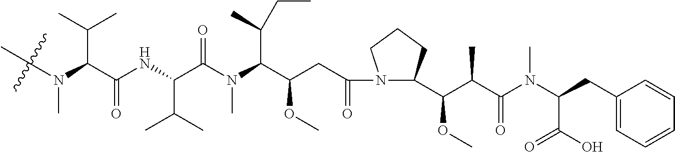
or a pharmaceutically acceptable salt form thereof; the DR5 binding agent is an anti-DR5 antibody attached to the Linker unit through a sulfur atom of the antibody; the Linker unit comprises a -Succinimide-Caproic acid-moiety; and the subscript p is an integer of from 1 to 8.

8. The ligand drug conjugate of claim 2, wherein LU has the formula:
-Aₐ-W_{w}—Y_{y}—
or a pharmaceutically acceptable salt form thereof, wherein,
-A- is a Stretcher unit;
the subscript a is 0 or 1;
each W is independently an amino acid unit;
the subscript w is an integer of from 0 to 12;
—Y— is a Spacer unit; and
the subscript y is 0, 1 or 2.

9. The ligand drug conjugate of claim 8, having the formula: 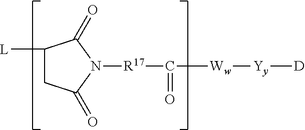
or a pharmaceutically acceptable salt form thereof, wherein
R¹⁷ is a member selected from the group consisting of —C₁-C₁₀ alkylene-, —C₂-C₁₀ alkenylene-, —C₂-C₁₀ alkynylene-, -carbocyclo-, —O—(C₁-C₈ alkylene)-, O—(C₂-C₈ alkenylene)-, —O—(C₂-C₈ alkynylene)-, -arylene-, —C₁-C₁₀ alkylene-arylene-, —C₂-C₁₀ alkenylene-arylene, —C₂-C₁₀ alkynylene-arylene, -arylene-C₁-C₁₀ alkylene-, -arylene-C₂-C₁₀ alkenylene-, -arylene-C₂-C₁₀ alkynylene-, —C₁-C₁₀ alkylene-(carbocyclo)-, —C₂-C₁₀ alkenylene-(carbocyclo)-, —C₂-C₁₀ alkynylene-(carbocyclo)-, -(carbocyclo)-C₁-C₁₀ alkylene-, -(carbocyclo)-C₂-C₁₀ alkenylene-, -(carbocyclo)-C₂-C₁₀ alkynylene, heterocyclo-, —C₁-C₁₀ alkylene-(heterocyclo)-, alkenylene-(heterocyclo)-, —C₂-C₁₀ alkynylene-(heterocyclo)-, -(heterocyclo)-C₁-C₁₀ alkylene-, -(heterocyclo)-C₂-C₁₀ alkenylene-, -(heterocyclo)-C₂-C₁₀ alkynylene-, —(CH₂CH₂O)ᵣ—, and —(CH₂CH₂O)ᵣ—CH₂—, wherein r is an integer of from 1 to 10, and wherein said alkyl, alkenyl, alkynyl, alkylene, alkenylene, alkynyklene, aryl, carbocyle, carbocyclo, heterocyclo, and arylene radicals, whether alone or as part of another group, are optionally substituted.

10. The ligand drug conjugate of claim 8, having the formula: 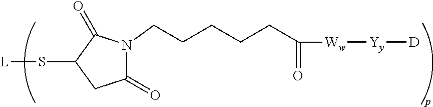
or a pharmaceutically acceptable salt form thereof, wherein S is a the sulfur atom provided by the Ligand unit (L).

11. The ligand drug conjugate of claim 8, having the formula: 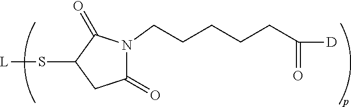
or a pharmaceutically acceptable salt form thereof, wherein S is a the sulfur atom provided by the Ligand unit (L).

12. The ligand drug conjugate of claim 8, having the formula: 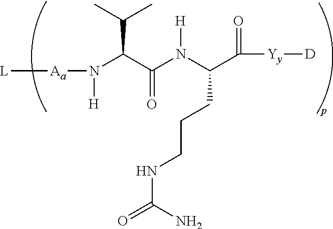
or a pharmaceutically acceptable salt form thereof.

13. The ligand drug conjugate of claim 8, having the formula: 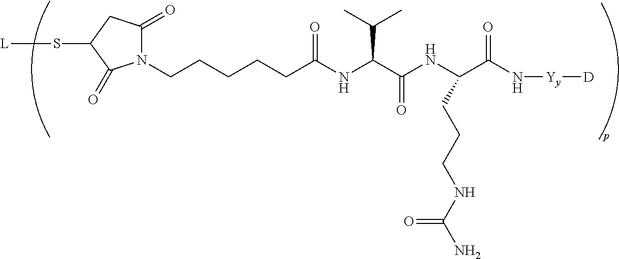
or a pharmaceutically acceptable salt form thereof.

14. The ligand drug conjugate of claim 8, having the formula: 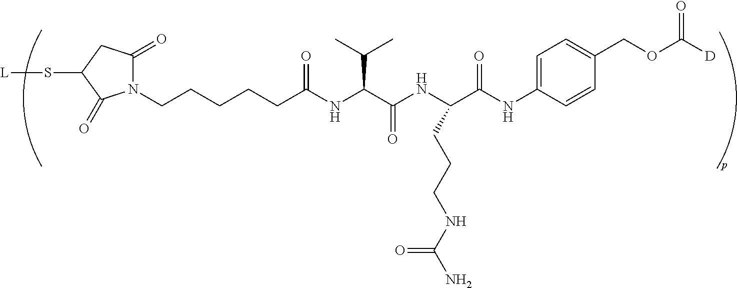
or a pharmaceutically acceptable salt form thereof.

15. The ligand drug conjugate of claim 8, wherein w is an integer ranging from 2 to 12, and y is 1 or 2.

16. The ligand drug conjugate of claim 8, wherein w is 2 and y is 1 or 2.

17. The ligand drug conjugate of claim 8, wherein W_{w} is -valine-citrulline- and y is 1 or 2.

18. A ligand drug conjugate having the formula: 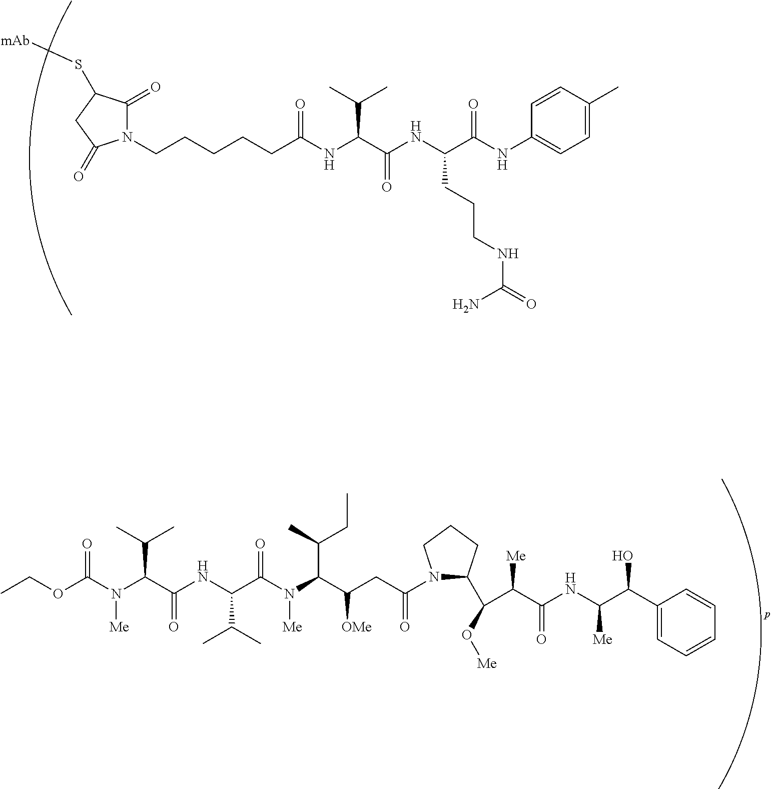
or pharmaceutically acceptable salt form thereof, wherein mAb is an anti-DR5 antibody, S is a sulfur atom of the antibody, and p is an integer of from 1 to 8.

19. A ligand drug conjugate having the formula: 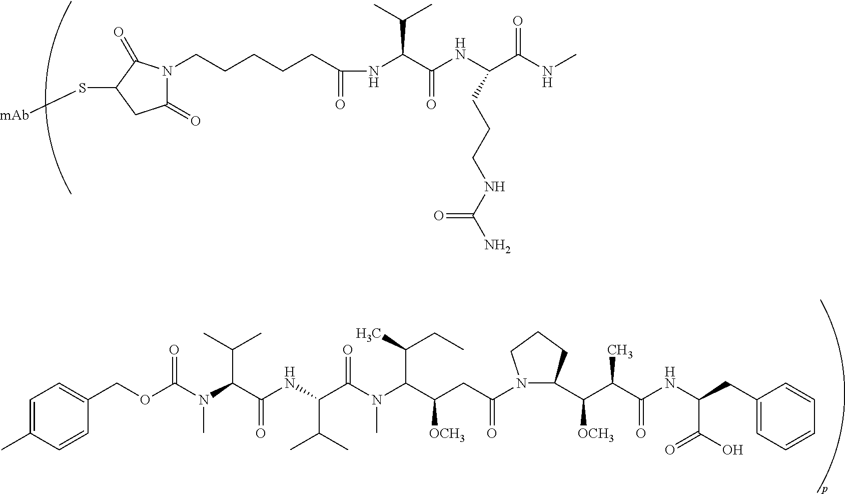
or pharmaceutically acceptable salt form thereof; wherein mAb is an anti-DR5 antibody, S is a sulfur atom of the antibody, and p is an integer of from 1 to 8

20. A ligand drug conjugate having the formula: 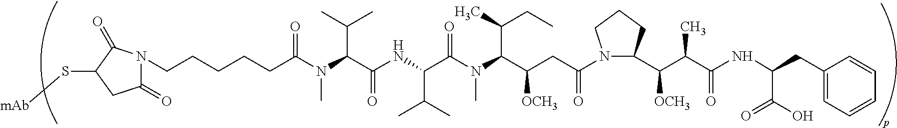
or pharmaceutically acceptable salt form thereof, wherein mAb is an anti-DRS antibody, S is a sulfur atom of the antibody, and p is an integer of from 1 to 8.

21. The ligand drug conjugate of claim 3, wherein D is D_{E}.

22. The ligand drug conjugate of claim 3, wherein D is D_{F}.

23. The ligand drug conjugate of claim 3, wherein L is an anti-DR5 antibody.

24. The ligand drug conjugate of any of claim 18, 19, or 20, wherein the anti-DR5 antibody comprises (a) a heavy chain immunoglobulin having the CDR1 consisting of amino residues 1-5 of SEQ ID NO: 3, the CDR2 consisting of amino acid residues 1-17 of SEQ ID NO:4, and the CDR3 consisting of amino acid residues 1-10 of SEQ ID NO:5; and (b) a light chain immunoglobulin having the CDR1 consisting of amino residues 1-11 of SEQ ID NO: 6, the CDR2 consisting of amino acid residues 1-7 of SEQ ID NO:7, and the CDR3 consisting of amino acid residues 1-8 of SEQ ID NO:8.

25. The ligand drug conjugate of any of claim 18, 19, or 20, wherein the anti-DR5 antibody comprises the heavy chain variable region comprising amino acid residues 1-118 of SEQ ID NO:1 and the light chain variable region comprising amino acid residues 1-107 of SEQ ID NO:2.

26. The ligand drug conjugate of any of claim 18, 19, or 20, wherein the anti-DR5 antibody comprises the heavy chain consisting of amino residues 1-449 of SEQ ID NO:1 and the light chain consisting of amino acid residues 1-213 of SEQ ID NO:2.

27. The ligand drug conjugate of claim 18, wherein p is from 3 to 5.

28. The ligand drug conjugate of claim 19, wherein p is from 1 to 3.

29. The ligand drug conjugate of claim 19, wherein p is from 3 to 5.

30. The ligand drug conjugate of claim 20, wherein p is from 3 to 5.

31. A pharmaceutical composition comprising the ligand drug conjugate of claim 1, in admixture with a pharmaceutically acceptable excipient.

32. An anti-tumor agent comprising the ligand drug conjugate of claim 1, as an effective ingredient.

33. A method of treating cancer comprising administering to a subject in need thereof an effective amount of a ligand drug conjugate of claim 1.

34. A method in accordance with claim 33, wherein said cancer is selected from the group consisting of melanoma, colorectal cancer, non-small cell lung carcinoma, uterine cancer, pancreatic cancer, prostate cancer, breast cancer, ovarian cancer, and hematological cancer.

35. A method in accordance with claim 33, wherein said cancer is pancreatic cancer.

36. A method in accordance with claim 33, wherein said cancer is melanoma.

37. A method in accordance with claim 33, wherein said cancer is breast cancer.
